(19) United States
(12) Patent Application Publication
(10) Pub. No.: US 2023/0181471 A1
(43) Pub. Date: Jun. 15, 2023

(54) CONCENTRATED COMPOSITIONS OF PROTEINS, THEIR PREPARATION AND USE THEREOF

(71) Applicant: HOFFMANN-LA ROCHE INC., Little Falls, NJ (US)

(72) Inventors: Naoual DAHMANA, Basel (CH); Samantha Veronica FACCHETTI, Basel (CH); Zana MARIN, Basel (CH); Sulabh Pravinchandra PATEL, Basel (CH); Gregoire SCHWACH, Basel (CH)

(21) Appl. No.: 17/998,613

(22) PCT Filed: Jul. 9, 2021
(86) PCT No.: PCT/EP2021/069090

§ 371 Date: Nov. 11, 2022

(30) Foreign Application Priority Data
Jul. 9, 2020 (EP) 20184997.3

(51) Int. Cl. A61K 9/16; A61K 47/68; A61K 9/00; A61K 9/10; A61K 9/19

(52) U.S. Cl. CPC A61K 9/1652; A61K 9/10; A61K 9/0019; A61K 9/19; A61K 47/6801

(57) ABSTRACT

The invention relates to a method for producing a composition comprising reversible protein complexes (RPCs), the method comprising the steps of contacting a protein and a complexing agent in a buffer solution, wherein the complexing agent is dextran sulphate or chondroitin sulphate, and wherein the protein and the complexing agent have opposite net charges when comprised in the buffer solution; formation of RPCs between the protein and the complexing agent in the buffer solution; and obtaining a suspension comprising the RPCs. Provided herein are also compositions, including pharmaceutical compositions/ formulations comprising the reversible protein complexes (RPCs) of the invention, in particular as obtained by the method provided herein.

### RELATED APPLICATIONS

The instant application is a 35 U.S.C. §371 filing of International Patent Application No. PCT/EP2021/069090, filed Jul. 9, 2021, which claims priority to European Patent Application No. 20184997.3, filed Jul. 9, 2020, the entire contents of which are incorporated herein by reference for all purposes.

### BACKGROUND OF THE INVENTION

In the last decades, biologics have been increasingly taking over small molecules in terms of FDA drug approvals. Indeed, while new biological entities (NBE) represented 10% of the total approved new molecular entities (NME) from 1993 to 1999, they raised up to 17% from 2000 to 2009 and to 24% from 2010 to 2019 (Mullard, Nature Reviews Drug Discovery, 2020, ISSN 1474-1784). This trend is attributed to the higher tolerability of biologics owing to their biological origin over the chemically synthetized small molecules but also for their higher selectivity, particularly the last antibody generations, procuring them a very specific targeting of the disease area, hence increasing safety and efficacy (Buvailo, https://www.biopharmatrend.com/post/67-will-small-molecules-sustain-pharmaceutical-race-with-biologics/; Jul. 11, 2018 ).

Biologics are predominantly commercialized as solutions for parenteral administration. They are formulated in an aqueous medium containing certain excipients necessary to ensure protein stability, prevent oxidation and ensure isotonicity. Biologics drug products are most frequently stored at 5±3° C. that results in a complicated and costly supply chain to maintain the cold chain, decreasing the drug accessibility; especially in the developing countries. In certain cases, protein solutions can be lyophilized in order to increase the stability and shelf-life of the drug product and enables easier transportation and storage compared to a cold chain. In this case, additional excipients such as cryoprotectants are used to protect the protein during the freeze-drying cycle and others to facilitate the reconstitution of the lyophilized formulation prior administration to the patient.

These standard dosage forms of biologics have been widely studied during these last decades and the scientific community witnessed the emergence of many limitations related to the current existing formulations. The first limitation is the particle formation in the biologic solutions, which is a major problem that the pharmaceutical industry is facing nowadays since these particles are considered immunogenic by the health authorities and hence may lead to product withdrawal which represent a considerable cost for the pharmaceutical industry. Recently, many efforts have been made to understand the mechanism underlying particle formation and results showed a surfactant-related origin; mainly polysorbates degradation and poloxamers interaction with silicon oils present in the primary packaging. Surfactants are among the excipients widely used in biologic formulations and are essential to prevent protein aggregation due to stresses experienced while processing, transporting and storage of the drug product (Gervasi et al., Eur J Pharm Biopharm, 2018, 131, 8-24). Hence, the pharmaceutical industry is aiming to find alternative excipients or ultimately, develop novel surfactant-free formulations of biologics.

Another limitation encountered by the current biologics formulations is the limitation in terms of concentration due to the high viscosity associated with the highly concentrated protein formulations. In parallel, lately, there is a clear trend in biologics formulation development when it comes to the route of administration by moving from intravenous infusion to subcutaneous administration to improve patient compliance as they can auto-inject the drug. To support this, formulations need to be highly concentrated (> 100 mg/mL) in order to allow the delivery of the therapeutic dose via a single injection. However, such highly concentrated formulations (>100 mg/mL) are associated with excessive viscosity complicating their administration via injection. Development of high concentration formulations of biologics may thus be very beneficial to improve the patient compliance.

In the past, reversible protein-polyion complexation concepts have been used to develop highly concentrated formulation of biologics and to tackle the aforementioned formulation challenges. Reversible protein complexes (RPCs), also referred to as hydrophobic ion pairing (HIP), protein-polyelectrolyte complexes (PPCs) or polyion complexes (PICs), have already been reported widely in the literature and was first introduced by Morawetz and Hughes in 1952 as a purification method, its use has since then evolved to other applications, including drug delivery systems for biologics (Mimura et al., J Chem Phys, 2019, 150(6), 064903; Morawetz and Hughes, The Journal of Physical Chemistry, 1952, 56(1), 64-49).

This concept relies on mixing oppositely charged molecules under specific physico-chemical conditions (e.g. pH, ionic strength, mole-charge ratio) leading to the formation of a complex via electrostatic interactions. Since the charges are neutralized and masked, the complex is not soluble anymore and precipitates as whitish particles. Importantly, this complexation is reversible by decreasing the electrostatic interactions, which are sensitive to the surrounding pH and ionic strength. Indeed, increasing the ionic strength causes an electrostatic shielding (or screening) between the oppositely charged molecules whereas changing the pH results in reducing the charge distribution on the molecules leading to decrease in the electrostatic interactions and consequent dissociation of the two molecules (Chamieh et al., In J Pharm, 2019, 559, 228-234; Matsuda et al., J Pharm Sci, 2018, 107(10), 2713-2719).

This concept has already been assessed with biological molecules; including enzymes, hormones, peptides and proteins, and an oppositely charged polymer for protein purification, dissolving enzymes in organic solvents without losing activity and also as a drug delivery strategy to enhance bioavailability Mimura et al., J Chem Phys, 2019, 150(6), 064903; Chamieh et al., In J Pharm, 2019, 559, 228-234; Matsuda et al., J Pharm Sci, 2018, 107(10), 2713-2719; Ristroph and Prud’homme, Nanoscale Advances, 2019, 1(11), 4207-4237). Indeed, most of the biological molecules are hydrophilic. The RPC concept was used to render them hydrophobic and increase their incorporation efficiency in already established drug delivery systems including micelles, liposomes and Self-emulsifying drug delivery systems (SEDDS) (Chamieh et al., In J Pharm, 2019, 559, 228-234). RPC has been shown to improve protein stability towards physico-chemical stress including heat, agitation and oxidation (Matsuda et al., J Pharm Sci, 2018, 107(10), 2713-2719).

It has been reported that to maximize the complexation efficiency, pH of the buffer should be adjusted to two pH units below or above the isoelectric point (pI) of the protein to maximize the charge distribution on the protein. Though, proteins are generally unstable at basic pH, thus mild acidic pH favoring positively charged protein is more suitable, and the oppositely charged polymer should be negatively charged to form a complex (Mimura et al., J Chem Phys, 2019, 150(6), 064903; Matsuda et al., J Pharm Sci, 2018, 107(10), 2713-2719).

It has also been mentioned that in addition to the electrostatic interactions between both polyions, hydrophobic interactions are involved in stabilizing the complex. Hence, another important parameter to take into consideration when selecting the complexing agent (CA) is the hydrophobicity. Indeed, the hydrophobic domains of the counterion such as an alkyl tail or aromatic group would coat the proteins’ surface area with hydrophobic domains that exclude water (Mimura et al., J Chem Phys, 2019, 150(6), 064903; Ristroph and Prud’homme, Nanoscale Advances, 2019, 1(11), 4207-4237).

While complexation of proteins can be efficiently achieved with a variety of complexing agents and under various conditions, subsequent dissociation of protein complexes still remains challenging. In particular, (partial) degradation of the protein or the formation of irreversible complexes negatively affects the dissociation efficiency. Especially when administered subcutaneously to a patient, compositions comprising protein complexes that do not efficiently dissociate at physiological conditions may pose a serious threat in terms of tolerability and risks of immunogenicity.

Accordingly, there is a need in the art for highly concentrated compositions comprising protein complexes that efficiently dissociate under physiological conditions into native, functional therapeutic proteins.

Thus, one objective of the present invention is to provide methods for producing compositions comprising reversible protein complexes that efficiently dissociate under physiological conditions.

A further objective of the invention is to provide compositions comprising reversible protein complexes that efficiently dissociate under physiological conditions.

A further objective of the invention is to provide pharmaceutical formulations comprising reversible protein complex for use as a medicament, in particular wherein the reversible protein complexes comprise a therapeutic protein and/or wherein the pharmaceutical formulation is administered subcutaneously.

### SUMMARY OF THE INVENTION

The technical problem is solved by the embodiments provided herein and as characterized in the claims. That is, the present invention relates, inter alia, to the following items:

1. A method for producing a composition comprising reversible protein complexes (RPCs), the method comprising the steps of:
a) contacting a protein and a complexing agent in a buffer solution,
wherein the complexing agent is dextran sulphate or chondroitin sulphate, and
wherein the protein and the complexing agent have opposite net charges when comprised in the buffer solution;
b) formation of RPCs between the protein and the complexing agent in the buffer solution; and
c) obtaining a suspension comprising the RPCs formed in step (b).

2. The method according to item 1, wherein the complexing agent is dextran sulphate, in particular dextran sulphate with 40 kDa molecular weight.

3. The method according to item 1 or 2, wherein the pH of the buffer solution is adjusted to be lower than the isoelectric point of the protein.

4. The method according to any one of items 1 to 3, wherein the pH of the buffer solution is adjusted to 2 to 5 pH units below the isoelectric point of the protein, in particular 3 pH units below the isoelectric point of the protein.

5. The method according to item 1 or 2, wherein the buffer solution has a pH ranging from 1 to 6, in particular wherein the buffer solution has a pH ranging from 3 to 6, in particular wherein the buffer solution has a pH ranging from 4.5 to 5.5.

6. The method according to any one of items 1 to 5, wherein the buffer solution has an ionic strength ranging from 20 to 50 mM, in particular wherein the buffer solution has an ionic strength ranging from 20 to 30 mM.

7. The method according to any one of items 1 to 6, wherein the buffer solution comprises histidine or citrate as buffering agent.

8. The method according to any one of items 1 to 7, wherein the buffer solution comprising the protein and the complexing agent is obtained by mixing a first solution comprising the protein and a second solution comprising the complexing agent.

9. The method according to item 8, wherein the first solution comprising the protein and/or the second solution comprising the complexing agent comprises a buffering agent.

10. The method according to any one of items 1 to 9, wherein the protein and the complexing agent are contacted at a mole-charge ratio ranging from 1:0.2 to 1:2, in particular wherein the protein and the complexing agent are contacted at a mole-charge ratio ranging from 1:0.2 to 1:1.

11. The method according to any one of items 1 to 10, wherein the protein is contacted with the complexing agent in the buffer solution at a protein concentration ranging from 1-40 mg/mL, in particular from 1-5 mg/mL.

12. The method according to any one of items 1 to 11, wherein the protein is an antibody, a growth factor, a hormone, a cytokine, an enzyme, or a fragment and/or fusion protein of any of the foregoing.

13. The method according to item 12, wherein the antibody is an antibody, in particular wherein the antibody is a monoclonal antibody, a polyclonal antibody, a chimeric antibody, a multispecific antibody, an antibody fusion protein, an antibody-drug-conjugate or an antibody fragment.

14. The method according to any one of items 1 to 13, wherein the complexing agent has a negative net charge when comprised in the buffer solution.

15. The method according to any one of items 1 to 14, wherein the complexing agent comprises a hydrophobic moiety.

16. The method according to any one of items 1 to 15, wherein the composition comprising the RPCs comprises at least one excipient.

17. The method according to item 16, wherein the at least one excipient is added to the composition before and/or after the formation of the RPCs.

18. The method according to item 17 or 18, wherein the at least one excipient is a stabilizer and/or a surfactant.

19. The method according to any one of items 1 to 18, wherein the method comprises a further step of exchanging the liquid fraction of the suspension comprising the RPCs.

20. The method according to item 19, wherein the liquid fraction of the suspension comprising the RPCs is exchanged by centrifugation of the suspension comprising the RPCs and resuspension of the sedimented RPCs in a buffer solution or water.

21. The method according to item 19, wherein the liquid fraction of the suspension comprising the RPCs is exchanged by dialysis of the suspension comprising the RPCs against a buffer solution or water.

22. The method according to any one of items 1 to 21, wherein the method comprises a further step of enriching the RPCs in the suspension to obtain an enriched RPC suspension.

23. The method according to item 22, wherein enriching the RPCs in the suspension comprises the steps of:
a) centrifuging the suspension comprising the RPCs to obtain a supernatant and a precipitate comprising an enriched RPC suspension; and
b) removing the supernatant from the precipitate to obtain an enriched RPC suspension.

24. The method according to item 22 or 23, wherein the liquid fraction of the enriched RPC suspension is at least in part replaced with a non-aqueous solvent during the enrichment step.

25. The method according to item 24, wherein the non-aqueous solvent is triacetin, diethylene glycol monoethyl ether or ethyl oleate.

26. The method according to any one of items 1 to 25, wherein the method comprises a further step of lyophilizing the suspension comprising the RPCs or the enriched RPC suspension to obtain a lyophilisate.

27. The method according to item 26, wherein at least one cryoprotectant is added to the suspension comprising the RPCs or the enriched RPC suspension before the lyophilisation step.

28. The method according to item 27, wherein the at least one cryoprotectant is selected from a group consisting of: sugars, amino acids, methylamines, lyotropic salts, polyols, propylene glycol, polyethylene glycol and pluronics.

29. The method according to any one of items 26 to 28, wherein the protein concentration of the suspension comprising the RPCs or the enriched RPC suspension is adjusted to 10 to 100 mg/mL, in particular to 40 to 80 mg/mL, prior to the lyophilisation step.

30. The method according to any one of items 1 to 25, wherein the method comprises a further step of spray drying the suspension comprising the RPCs or the enriched RPC suspension to obtain a spray dried powder.

31. The method according to item 30, wherein the protein concentration of the suspension comprising the RPCs or the enriched RPC suspension is adjusted to 1 to 10 mg/mL, in particular to 1 to 5 mg/mL, prior to the spray drying step.

32. The method according to item 30 or 31, wherein the liquid fraction of the suspension comprising the RPCs or the enriched RPC suspension is exchanged prior to the spray drying step.

33. The method according to item 32, wherein exchanging the liquid fraction of the suspension comprising the RPCs or the enriched RPC suspension reduces the concentration of at least one buffering agent, complexing agent and/or excipient in the suspension.

34. The method according to item 33, wherein the suspension comprising the RPCs or the enriched RPC suspension is substantially free of buffering agent after exchanging the liquid fraction of the suspension.

35. The method according to item 33 or 34, wherein the liquid fraction of the suspension is exchanged before the spray-drying step to obtain a mole-charge ratio between the protein and the complexing agent between 1:0.2 to 1:1, in particular between 1:0.4 to 1:0.8.

36. The method according to any one of items 30 to 35, wherein spray drying is performed at an inlet temperature 115° C. and/or an outlet temperature of 48° C.

37. The method according to any one of items 30 to 36, wherein spray drying is performed at a feed rate of 17 mL/min.

38. The method according to any one of items 30 to 37, wherein the method comprises a further step of resuspending the spray dried powder in a non-aqueous solvent (NAS) to obtain an RPC-NAS suspension.

39. The method according to item 38, wherein the non-aqueous solvent is at least one selected from a group consisting of: diethylene glycol monoethyl ether, ethyl oleate, triacetin, isosorbide dimethyl ether and glycofurol.

40. The method according to item 39, wherein the spray dried powder is resuspended to obtain a RPC-NAS suspension with a protein concentration ranging from 50 to 300 mg/mL, in particular ranging from 100 - 250 mg/mL.

41. A composition comprising reversible protein complexes (RPCs), wherein the composition is obtained by the method according to any one of items 1 to 40.

42. A composition comprising reversible protein complexes (RPCs), wherein the RPCs comprise a protein and a complexing agent, and

wherein the complexing agent is dextran sulphate or chondroitin sulphate.

43. The composition according to item 42, wherein the complexing agent is dextran sulphate, in particular dextran sulphate with 40 kDa molecular weight.

44. The composition according to item 42 or 43, wherein the protein has a positive net charge when comprised in the RPCs.

45. The composition according to item 44, wherein the protein is an antibody, a growth factor, a hormone, a cytokine, an enzyme, or a fragment and/or fusion protein of any of the foregoing.

46. The composition according to item 45, wherein the antibody is a monoclonal antibody, a polyclonal antibody, a chimeric antibody, a multispecific antibody, an antibody fusion protein, an antibody-drug-conjugate or an antibody fragment.

47. The composition according to any one of items 42 to 46, wherein the complexing agent has a negative charge when comprised in the RPCs.

48. The composition according to any one of items 42 to 47, wherein the complexing agent comprises a hydrophobic moiety.

49. The composition according to any one of items 42 to 48, wherein the composition comprises at least one excipient.

50. The composition according to item 49, wherein the at least one excipient is a stabilizer and/or a surfactant.

51. The composition according to any one of items 42 to 50, wherein the protein has a higher melting temperature when comprised in the RPC compared to the uncomplexed protein.

52. The composition according to any one of items 42 to 51, wherein the RPCs comprising the protein and the complexing agent dissociate at physiological pH and ionic strength.

53. The composition according to any one of items 42 to 51, wherein the RPCs comprising the protein and the complexing agent dissociate in 10 mM to 100 mM PBS (pH 7.4, 137 mM NaCl) when diluted to a protein concentration of 0.1 to 10 mg/mL.

54. The composition according to any one of items 42 to 53, wherein the composition is a suspension.

55. The composition according to item 54, wherein the suspension is obtained with the method according to any one of items 1 to 25.

56. The composition according to items 54 or 55, wherein the protein concentration in the suspension ranges from 50 to 250 mg/mL, in particular wherein the protein concentration in the suspension ranges from 100 to 200 mg/mL.

57. The composition according to any one of items 54 to 56, wherein the suspension comprises uncomplexed complexing agent.

58. The composition according to any one of items 54 to 57, wherein the RPCs comprised in the suspension have a mean particle size ranging from 5 to 20 µm, in particular wherein the RPCs comprised in the suspension have a mean particle size ranging from 6 to 12 µm.

59. The composition according to any one of items 54 to 57, wherein the RPCs comprised in the suspension have a mean particle size ranging from 100 to 4000 nm, in particular wherein the RPCs comprised in the suspension have a mean particle size ranging from 150 to 2000 nm.

60. The composition according to any one of items 54 to 57, wherein the RPCs comprised in the suspension have a mean particle size ranging from 0.1 to 20 µm, in particular wherein the RPCs comprised in the suspension have a mean particle size ranging from 0.1 to 12 µm.

61. The composition according to any one of items 54 to 60, wherein the suspension is injectable through a 26G needle.

62. The composition according to any one of items 54 to 61, wherein the suspension is stable for at least 4 weeks at 4° C. and/or 25° C.

63. The composition according to any one of items 54 to 62, wherein the suspension has a viscosity ranging from 2 to 20 cP, in particular ranging from 3 to 15 cP, when measured at 20° C.

64. The composition according to any one of items 54 to 63, wherein the pH of the suspension is lower than the isoelectric point of the protein.

65. The composition according to any one of items 54 to 64, wherein the pH of the suspension is 1 to 3 pH units lower than the isoelectric point of the protein, in particular wherein the pH of the suspension is 2 pH units lower than the isoelectric point of the protein.

66. The composition according to any one of items 54 to 64, wherein the pH of the suspension ranges from 1 to 6, in particular wherein the pH of the suspension ranges from 4.5 to 5.5.

67. The composition according to any one of items 54 to 66, wherein the suspension comprises a buffering agent.

68. The composition according to item 67, wherein the buffering agent is histidine or citrate.

69. The composition according to any one of items 54 to 68, wherein the suspension has an ionic strength ranging from 20 to 50 mM, in particular wherein the suspension has an ionic strength ranging from 20 to 30 mM.

70. The composition according to any one of items 54 to 66, wherein the suspension is substantially free of buffering agents.

71. The composition according to any one of items 54 to 70, wherein the suspension further comprises a non-aqueous solvent.

72. The composition according to item 71, wherein the non-aqueous solvent is diethylene glycol monoethyl ether, triacetin or ethyl oleate.

73. The composition according to any one of items 42 to 53, wherein the composition is a lyophilisate.

74. The composition according to item 73, wherein the lyophilisate is obtained with the method according to any one of items 26 to 29.

75. The composition according to item 73 or 74, wherein the lyophilisate comprises a buffering agent.

76. The composition according to item 75, wherein the buffering agent is histidine or citrate.

77. The composition according to any one of items 73 to 76 wherein the lyophilisate comprises at least one cryoprotectant.

78. The composition according to item 77, wherein the at least one cryoprotectant is selected from a group consisting of: sugars, amino acids, methylamines, lyotropic salts, polyols, propylene glycol, polyethylene glycol and pluronics.

79. The composition according to any one of items 73 to 78, wherein the lyophilisate is stable for at least 4 weeks at 40° C.

80. The composition according to any one of items 73 to 79, wherein the lyophilisate is reconstituted in a liquid to a protein concentration ranging from 50 to 250 mg/mL, in particular wherein the lyophilisate is reconstituted in a liquid to a protein concentration ranging from 100 to 200 mg/mL.

81. The composition according to item 80, wherein the liquid is PBS.

82. The composition according to item 80 or 81, wherein the resuspended lyophilisate has a viscosity ranging from 2 to 20 cP, in particular ranging from 10 to 20 cP.

83. The composition according to any one of items 42 to 53, wherein the composition is a spray dried powder.

84. The composition according to item 83, wherein the protein content of the spray dried powder is at least 40% by weight (w/w), at least 50% by weight (w/w), at least 60% by weight (w/w)

85. The composition according to item 83 or 84, wherein the spray dried powder is obtained with the method according to any one of items 30 to 37.

86. The composition according to any one of items 83 to 85, wherein the spray dried powder comprises a buffering agent.

87. The composition according to item 86, wherein the buffering agent is histidine or citrate.

88. The composition according to any one of items 83 to 85, wherein the spray dried powder is substantially free of buffering agents.

89. The composition according to any one of items 83 to 88, wherein the RPCs comprised in the spray dried powder have a mean particle size ranging from 5 to 50 µm, in particular ranging from 10 to 40 µm, in particular ranging from 20 to 35 µm.

90. The composition according to any one of items 83 to 89, wherein the spray dried powder is re-suspended in a liquid to a protein concentration in the suspension ranging from 50 to 300 mg/mL, in particular wherein the spray dried powder is re-suspended in a liquid to a protein concentration in the suspension ranging from 100 to 250 mg/mL.

91. The composition according to item 90, wherein the liquid is a non-aqueous solvent.

92. The composition according to item 91, wherein the non-aqueous solvent is at least one selected from a group consisting of: diethylene glycol monoethyl ether, ethyl oleate, triacetin, isosorbide dimethyl ester and glycofurol, preferably diethylene glycol monoethyl ether, ethyl oleate or triacetin.

93. The composition according to any one of items 90 to 92, wherein the reconstituted spray dried powder has a viscosity ranging from 10 to 100 cP, in particular ranging from 20 to 80 cP.

94. A pharmaceutical formulation comprising the composition according to any one of items 41 to 93.

95. The pharmaceutical formulation according to item 94, wherein the pharmaceutical formulation comprises the suspension according to any one of items 54 to 72, the reconstituted lyophilisate according to any one of items 80 to 82, or the re-suspended spray dried powder according to any one of items 90 to 93.

96. The pharmaceutical formulation according to items 94 or 95 for use as a medicament.

97. The pharmaceutical formulation according to any one of items 94 to 96 for use in the treatment of an autoimmune disease, an immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, or atherosclerosis.

98. The pharmaceutical formulation for use according to item 97, wherein the pharmaceutical formulation is administered subcutaneously, intramuscularly, transdermally, ocullarly, such as subconjunctivally, intracamerally, intravitreally, subretinally, or suprachoroidally, to the brain, such as intralumbarly, intrathecally, or intraventricularly, intra-articularly, or by inhalation.

99. Use of the pharmaceutical formulation according to items 94 or 95 for the treatment of a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis.

100. Use of the pharmaceutical formulation according to items 94 or 95 in the preparation of a medicament for the treatment of a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis.

101. A method of treating a subject suffering from a disease selected from the group consisting of: an autoimmune disease, an immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis, the method comprising the steps of (a) producing the pharmaceutical formulation according items 94 or 95; and (b) administering the pharmaceutical formulation to a subject in need thereof.

102. The method according to item 101, wherein the pharmaceutical composition is administered subcutaneously, intramuscularly or transdermally, in particular wherein the pharmaceutical composition is administered subcutaneously.

103. A method of subcutaneous, intramuscular or transdermal administration of a pharmaceutical formulation, the method comprising the steps of (a) producing the pharmaceutical formulation according to items 94 or 95; and (b) administering the pharmaceutical formulation to a subject by subcutaneous, intramuscular or transdermal delivery.

Accordingly, in one aspect, the invention relates to a method for producing a composition comprising reversible protein complexes (RPCs), the method comprising the steps of: (a) contacting a protein and a complexing agent in a buffer solution, wherein the complexing agent is dextran sulfate and/or chondroitin sulfate, and wherein the protein and the complexing agent have opposite charges when comprised in the buffer solution, preferably resulting in a mole-charge ratio of protein to complexing agent of about 1:1 or higher than 1:1; (b) formation of RPCs between the protein and the complexing agent in the buffer solution; and (c) obtaining a suspension comprising the RPCs formed in step (b).

That is, the invention is based at least in part on the surprising finding that the complexing agents dextran sulfate and chondroitin sulfate can be used to produce high concentration protein compositions comprising reversible protein complexes (RPCs) that efficiently dissolve under physiological conditions. Further, it was unexpectedly found that formulations comprising the RPCs of the invention have a particularly low viscosity even at high protein concentrations and are thus suitable for subcutaneous administration.

It has been demonstrated by the inventors that both dextran sulfate and chondroitin sulfate can be used to complex proteins with nearly 100% efficiency (FIG. 9) Surprisingly, RPCs comprising these complexing agents dissolve with nearly 100% efficiency in PBS (pH 7.4, 137 mM NaCl), which simulates physiological conditions in humans. Thus, formulations comprising the RPCs of the invention can be administered to a patient, preferably subcutaneously, where they dissolve in situ such that the protein becomes available to the patient.

Producing the reversible protein complexes of the invention requires a first step of contacting the protein and the complexing agent in a buffer solution. That is, the protein and the complexing agent may be mixed in a buffer solution such that a reversible protein complex forms between the protein and the complexing agent. Without being bound to theory, formation of the reversible protein complex is at least partially driven by electrostatic interactions between the oppositely charged proteins and complexing agents in the buffer solution. It is to be understood that a single protein molecule usually undergoes complex formation with multiple molecules of the complexing agent.

That is, within the present invention, the complexing agent and the protein have opposite charges when comprised in the buffer solution. Thus, in certain embodiments, the complexing agent may be positively charged, and the protein may be negatively charged when comprised in the buffer solution. However, it is preferred that the protein is positively charged when comprised in the buffer solution and that the complexing agent is negatively charged when comprised in the buffer solution.

It is to be understood that both the protein and the complexing agent may be dissolved in the buffer solution before the contacting step. Contacting of the oppositely charged proteins and complexing agents in the buffer solution results in precipitation of reversible protein complexes, thereby turning the buffered solution into a suspension comprising the reversible protein complexes.

A “composition”, as used herein, refers to a mixture comprising the reversible protein complexes of the invention and at least one further compound. That is, the composition of the invention preferably comprises reversible protein complexes comprising the complexing agents dextran sulfate and/or chondroitin sulfate. The composition may have any form. However, in certain embodiments, the composition is a suspension. In other embodiments, the composition is a powder, in particular a lyophilized powder or a spray dried powder, or a reconstituted or re-suspended powder.

In certain embodiments, the composition of the invention is a suspension. The term “suspension” as used herein refers to a dispersion with continuous liquid phase and a discontinuous solid phase in form of particles, such as RPCs, that have a number average diameter of 5 to 300 µm. However, it is important to understand that the term “suspension” may also encompass dispersions comprising smaller particles, such as RPCs having a number average diameter in the nanometer range.

The term “complex” as used herein refers to the association of two or more molecules, usually by non-covalent bonding, e.g., the association between a positively charged group of a first molecule and a negatively charged group of a second molecule. Further, complex formation may be facilitated by hydrophobic interactions between the first molecule and the second molecule. Within the present invention, the first molecule is preferably a protein and the second molecule is preferably a complexing agent.

A complex is said to be reversible, if the association between the two molecules, i.e., the protein and the complexing agent, can be reversed without significantly modifying the protein. That is, a protein complex is said to be reversible, if the protein complex can dissociate and the protein retains its original size, structure and/or function after dissociation of the complex.

Preferably, a protein complex is determined to be a reversible protein complex, if both the formation of the complex and the dissociation of the complex can be achieved with high efficiency.

The term “complexing efficiency”, as used herein, refers to the efficiency with which a complex involving a protein and a complexing agent is formed under a specific condition, i.e., a specific pH and/or ionic strength. Complexing efficiency is defined as the percentage of proteins in a sample that have undergone complex formation after contacting with a complexing agent. In particular, a complex is said to form with high complexing efficiency, if at least 85%, at least 90%, at least 91%, at least 92%, at least 93%, at least 94%, at least 95%, at least 96%, at least 97%, at least 98%, at least 99% of the proteins in a sample have undergone complex formation following a contacting step with a complexing agent.

The term “dissociation efficiency”, as used herein, refers to the efficiency with which a complex involving a protein and a complexing agent is dissociated under a specific condition, i.e., a specific pH and/or ionic strength. Dissociation efficiency is defined as the percentage of protein complexes in a sample that dissolved under the specific conditions such that the protein is released from the complex and goes back in solution. In particular, a complex is said to dissociate with a high dissociation efficiency, if at least 85%, at least 90%, at least 91%, at least 92%, at least 93%, at least 94%, at least 95%, at least 96%, at least 97%, at least 98%, at least 99% of the complexes in a sample dissociate into soluble proteins under the specific conditions in the sample.

Thus, a protein complex is defined to be a “reversible protein complex”, if the complex is formed with a high complexing efficiency and dissolves with a high dissociation efficiency.

Further, a protein complex is defined to be a “reversible protein complex”, if the protein retains its original size, structure and/or function after dissociation of the protein complex. In particular, a protein complex is determined to be a reversible protein complex, if at least 70%, at least 75%, at least 80%, at least 85%, at least 90%, at least 91%, at least 92%, at least 93%, at least 94%, at least 95% of the proteins in a sample retain their original size, structure and/or function after they have been released from the protein complex in the dissociation step.

Preferably, a protein complex is determined to be a reversible protein complex, if at least 70%, at least 75%, at least 80%, at least 85%, at least 90%, at least 91%, at least 92%, at least 93%, at least 94%, at least 95% , at least 96%, at least 97%, at least 98%, at least 99% of the proteins in a sample retain their original size, structure and/or function after dissociation of the protein complex in 10 - 100 mM PBS (pH 7.4).

More preferably, a protein complex is determined to be a reversible protein complex, if at least 70%, at least 75%, at least 80%, at least 85%, at least 90%, at least 91%, at least 92%, at least 93%, at least 94%, at least 95%, at least 96%, at least 97%, at least 98%, at least 99% of the proteins in a sample retain their original size, structure and/or function after dissociation of the protein complex under physiological conditions.

The skilled person is aware of methods to determine complexing efficiency and dissociation efficiency. For example, methods to determine complexing efficiency are disclosed in Example 1.2.3 and methods to determine dissociation efficiency are disclosed in Example 1.2.4, respectively. Further, the skilled person is aware of methods to determine if a protein retains its original size, structure and/or function after dissociation of the protein complex.

Changes in the size of a protein mainly result from degradation of the protein, which commonly results in reduced protein size, or aggregation of two or more protein, which commonly results in increased protein size. For example, the size of a protein may be determined before formation of the complex and after dissociation of the complex by any method known in the art, such as size exclusion chromatography (SEC) or ion exchange chromatography (IEC) (see Example 1.2.8).

That is, in certain embodiments, a protein is said to be stable if, after dissociation of RPCs in a sample, at least 70%, at least 75%, at least 80%, at least 85%, at least 90%, at least 91%, at least 92%, at least 93%, at least 94%, at least 95% of the proteins are in monomeric form and not degraded, preferably when measured by size exclusion chromatography (SEC).

Alternatively, a protein is said to be stable, if the main peak percentage of the protein differs by not more than 1%, 2%, 3%, 4%, 5%, 6%, 7%, 8%, 9%, 10%, 11%, 12%, 13%, 14%, 15%, 16%, 17%, 18%, 19%, 20% when analyzed by ion exchange chromatography (IEC) and compared before the formation of the RPC and after dissociation of the RPC.

The structure of a protein may be determined before formation of the complex and after dissociation of the complex by any method known in the art, such as X-ray crystallography, NMR or circular dichroism.

The function of a protein may be determined before formation of the complex and after dissociation of the complex. For example, the function of an antigen-binding molecule, such as an antibody, may be determined in binding studies. Common methods known in the art to determine the binding of a protein to a target are isothermal titration calorimetry or surface plasmon resonance. Preferably, the antigen-binding molecule may have substantially the same binding characteristics after dissociation of the complex as it had before the formation of the complex. That is, the antigen-binding molecule is said to have substantially the same binding characteristics, if the binding affinity of the antigen-binding molecule for the antigen differs less than 10%, less than 9%, less than 8%, less than 7%, less than 6%, less than 5% when measured before complex formation and after dissociation of the complex

The term “contacting” as used in the context of the methods of the present invention is understood by the skilled person. The term relates to bringing two compounds of the present invention, i.e., a protein and a complexing agent, in physical contact with each other. The protein and the complexing agent are preferably brought into physical contact in a buffer solution. That is, both the protein and the complexing agent are dissolved in a buffer solution such that they can get into physical contact by means of diffusion. Contacting of the compounds in the buffer solution may be facilitated by shaking, mixing, vortexing or the like.

The term “electrostatic interactions” as used herein, refers to interactions that are formed between two substances that have opposite charges, namely, a positively charged substance and a negatively charged substance. Such interactions typically involve ionic bonds.

Most proteins both contain acidic and basic functional groups. Thus, a protein is said to be negatively charged if the net charge of the protein is negative under the given conditions. Accordingly, a protein is said to be positively charged if the net charge of the protein is positive under the given conditions. The net charge of a polypeptide at a given pH can be calculated on the basis of the Henderson-Hasselbalch equation (Hasselbalch, K. A., 1917 Biochemische Zeitschrift 78: 112-144) and known pKa values of ionisable amino acid side chains and the N-and C-termini of a polypeptide.

The term “buffer solution”, as used herein, refers to a composition, wherein the composition comprises a weak acid and its conjugate base (usually as a conjugate base salt), a weak base and its conjugate acid, or mixtures thereof. Those skilled in the art would readily recognize a variety of buffer solutions that could be used in the methods and/or formulations used in the invention. Typical buffer solutions include, but are not limited to pharmaceutically acceptable weak acids, weak bases, or mixtures thereof.

The phrase “weak acid” is a chemical acid that does not fully ionize in aqueous solution; that is, if the acid is represented by the general formula HA, then in aqueous solution A⁻ forms, but a significant amount of undissociated HA still remains. The acid dissociation constant (Kα) of a weak acid varies between 1.8×10⁻¹⁶ and 55.5.

The phrase “weak base” is a chemical base that does not fully ionize in aqueous solution; that is, if the base was represented by the general formula B, then in aqueous solution BH⁺ forms, but a significant amount of unprotonated B still remains. The acid dissociation constant (Kα) of the resultant conjugate weak acid BH⁺ varies between 1.8×10⁻¹⁶ and 55.5.

The phrase “conjugate acid” is the acid member, HX⁺, of a part of two compounds (HX⁺, X) that transform into each other by gain or loss of a proton.

The phrase “conjugate base” is the base member, X⁻, of a pair of two compounds (HX, X⁻) that transform into each other by gain or loss of a proton.

The phrase “conjugate base salt” is the ionic salt comprising a conjugate base, X⁻, and a positively charged counter-ion.

Within the present invention, the complexing agent may be a polymer, preferably a charged polymer. That is, the present invention is preferably based on the formation of reversible protein complexes between proteins and polymers, wherein the polymers have an opposite charge compared to the net charge of the protein when comprised in the buffer solution.

In particular, it has been demonstrated within the present invention that polymeric complexing agents result in reversible protein complexes that dissolve more efficiently at physiological conditions compared to complexes that have been formed with monomeric complexing agents such as sodium dodecyl sulfate (SDS) or sodium taurocholate (ST) (see FIG. 9). In particular, it has been demonstrated that monomeric complexing agents such as SDS or ST result in significant degradation of the protein after dissociation of the complex (see Table 8).

In certain embodiments, the complexing agent is dextran sulfate. It has been shown by the inventors that dextran sulfate can be used to form reversible protein complexes that efficiently dissociate at physiological conditions. Thus, in a particular embodiment, the invention relates to the method according to the invention, wherein the complexing agent is dextran sulfate.

The term “dextran sulfate” as used herein refers a polyanionic derivative of dextran, ranging in molecular weight from 7,000 to 500,000 daltons. Dextrans are polymers of glucose in which glucose residues are joined by α-1,6 linkages. Thus, in certain embodiments, the invention relates to the method according to the invention, wherein the complexing agent is dextran sulfate with an average molecular weight ranging from 7,000 to 500,000 daltons.

Dextran sulfate with an average molecular weight of 40 kDa was shown to be particularly suitable for the formation of reversible protein complexes that efficiently dissociate at physiological conditions. Thus, in a preferred embodiment, the invention relates to the method according to the invention, wherein the complexing agent is dextran sulfate with an average molecular weight ranging from 10 kDa to 200 kDa. In a more preferred embodiment, the invention relates to the method according to the invention, wherein the complexing agent is dextran sulfate with an average molecular weight ranging from 20 kDa to 100 kDa. In an even more preferred embodiment, the invention relates to the method according to the invention, wherein the complexing agent is dextran sulfate with an average molecular weight ranging from 20 kDa to 80 kDa. In an even more preferred embodiment, the invention relates to the method according to the invention, wherein the complexing agent is dextran sulfate with an average molecular weight ranging from 30 kDa to 50 kDa. In a most preferred embodiment, the invention relates to the method according to the invention, wherein the complexing agent is dextran sulfate with an average molecular weight of 40 kDa.

In other embodiments, the complexing agent is chondroitin sulfate. Chondroitin sulfate is a sulfated glycosaminoglycan (GAG) composed of a chain of alternating sugars (N-acetylgalactosamine and glucuronic acid). Within the present invention, the term “chondroitin sulfate” encompasses chondroitin sulfate A (chondroitin-4-sulfate), chondroitin sulfate C (chondroitin-6-sulfate), chondroitin sulfate D (chondroitin-2,6-sulfate) and chondroitin sulfate E (chondroitin-4,6-sulfate). In certain embodiments, the invention relates to the method according to the invention, wherein the complexing agent is chondroitin sulfate A. In other embodiments, the invention relates to the method according to the invention, wherein the complexing agent is chondroitin sulfate C. In further embodiments, the invention relates to the method according to the invention, wherein the complexing agent is chondroitin sulfate D. In further embodiments, the invention relates to the method according to the invention, wherein the complexing agent is chondroitin sulfate E. In further embodiments, the invention relates to the method according to the invention, wherein the complexing agent is a at least one of the group consisting of chondroitin sulfate A, chondroitin sulfate C, chondroitin sulfate D and chondroitin sulfate E.

The term “charged polymer” refers to any compound composed of a backbone of repeating structural units linked in linear or non-linear fashion, some of which repeating units contain positively or negatively charged chemical groups. The repeating structural units may be polysaccharide, hydrocarbon, organic, or inorganic in nature. The repeating units may range from n=2 to n=several million.

The term “positively charged polymer” as used herein refers to polymers containing chemical groups which carry, can carry, or can be modified to carry a positive charge such as ammonium, alkyl ammonium, dialkylammonium, trialkyl ammonium, and quaternary ammonium.

The term “negatively charged polymer” as used herein refers to polymers containing chemical groups which carry, can carry, or can be modified to carry a negative charge such as derivatives of phosphoric and other phosphorous containing acids, sulfuric and other sulfur containing acids, nitrate and other nitrogen containing acids, formic and other carboxylic acids.

In a particular embodiment, the invention relates to the method according to the invention, wherein the pH of the buffer solution is adjusted to be lower than the isoelectric point of the protein.

Formation of reversible protein complexes is mainly driven by electrostatic interaction between the oppositely charged proteins and complexing agents that form the complex. The charge of a molecule in a solution depends, amongst others, on the pH of the solution. Within the present invention, it is preferred that the protein and the complexing agent are comprised in a buffer solution, wherein the pH of the buffer solution is adjusted such that the protein and the complexing agent are oppositely charged.

Proteins may comprise positively and negatively charged amino acid residues when comprised in a solution. If a protein comprises more negative charges than positive charges under a specific condition, the protein is said to have a negative net charge under said condition. If a protein comprises more positive charges than negative charges under a specific condition, the protein is said to have a positive net charge under said condition.

The term “isoelectric point” as used herein means the pH value where the overall net charge of a macromolecule such as a protein is zero. In proteins there may be many charged groups, and at the isoelectric point the sum of all these charges is zero. At a pH above the isoelectric point the overall net charge of the polypeptide will be negative, whereas at pH values below the isoelectric point the overall net charge of the polypeptide will be positive.

The skilled person is aware of methods to determine the isoelectric point of a protein. Most commonly, the isoelectric point of a protein is computed based on the amino acid sequence of the protein. Numerous tools are available online that allow computing the isoelectric point of a protein, such as “ExPASy Compute pI/Mw”; see Protein Identification and Analysis Tools on the ExPASy Server; Gasteiger E., Hoogland C., Gattiker A., Duvaud S., Wilkins M.R., Appel R.D., Bairoch A.; (In) John M. Walker (ed): The Proteomics Protocols Handbook, Humana Press (2005), pp. 571-607.

The protein forming the reversible protein complex may have a positive or negative net charge. However, it is preferred that the protein has a positive net charge when comprised in the buffer solution. That is, it is preferred that the pH of the buffer solution is lower than the isoelectric point of the protein.

It is commonly observed that exposure to strongly acidic pH may result in irreversible denaturation of proteins. Thus, it is preferred that the pH of the buffer solution is adjusted to a pH that is 2 to 5 pH units below the isoelectric point of the protein. Thus, in a particular embodiment, the invention relates to the method according to the invention, wherein the pH of the buffer solution is adjusted to 2 to 5 pH units below the isoelectric point of the protein. In a preferred embodiment, the invention relates to the method according to the invention, wherein the pH of the buffer solution is adjusted to about 3 pH units below the isoelectric point of the protein.

Adjusting the pH of the buffer solution to a pH that is only slightly below the isoelectric point of the protein ensures that the protein is positively charged when comprised in the buffer solution and, at the same time, reduces the risk of protein denaturation.

Alternatively, the pH of the buffer solution may be adjusted to a fixed value. The method of the present invention is preferably used for the production of reversible protein complexes comprising therapeutic proteins, i.e. antibodies. Most antibodies have an isoelectric point ranging from 6.5 - 9. Thus, most antibodies will have a positive net charge when comprised in a buffer solution with an acidic pH value. Thus, in a particular embodiment, the invention relates to the method according to the invention, wherein the buffer solution has a pH ranging from 1 to 6. In a preferred embodiment, the invention relates to the method according to the invention, wherein the buffer solution has a pH ranging from 3 to 6. In a more preferred embodiment, the invention relates to the method according to the invention, wherein the buffer solution has a pH ranging from 4.5 to 5.5.

It has been shown by the inventors that complex formation requires a buffer solution with an ionic strength of at least 5 mM, preferably at least 20 mM (FIG. 13). Thus, in a particular embodiment, the invention relates to the method according to the invention, wherein the buffer solution has an ionic strength ranging from 5 to 50 mM, preferably 20 to 50 mM. In a preferred embodiment, the invention relates to the method according to the invention, wherein the buffer solution has an ionic strength ranging from 20 to 30 mM.

The term “ionic strength” is used herein as the following function of the concentration of all ions in a solution:

$$I = \frac{1}{2} {\sum }_{i = 1}^{n} {c}_{i} {z}_{i}^{2}$$

wherein cᵢ is the molar concentration of ion i (M, mol/L), zᵢ is the charge number of that ion and the sum is taken over all ions in the solution.

The buffer solution may comprise any buffering agent that allows efficient reversible protein complex formation. Many buffering agents are known in the art that allow to maintain the pH of a solution near a chosen pH value. Buffering agents that may be used for the method of the present invention include, without limitation, formate, citrate, succinate, acetate, propionate, malate, pyridine, piperazine, cacodylate, succinate, MES, maleate, histidine, bis-tris, phosphate, ethanolamine, ADA and carbonate.

It is to be understood that the choice of the buffering agent depends on the protein that is to be complexed. In certain embodiments, the buffer solution is adjusted to a pH that is below the isoelectric point of the protein that is to be complexed. The skilled person is aware of methods to determine the isoelectric point of a protein and to select a buffering agent that allows to maintain a pH value in the buffer solution that is below the isoelectric point of said protein. Thus, in certain embodiments, the invention relates to the method of the invention, wherein the buffer solution comprises a buffering agent that allows to maintain the pH of the buffer solution below the isoelectric point of the protein. In a particular embodiment, the invention relates to the method according to the invention, wherein the buffer solution comprises a buffering agent that allows to maintain the pH of the buffer solution 1 to 3 pH units below the isoelectric point of the protein. In a particular embodiment, the invention relates to the method according to the invention, wherein the buffer solution comprises a buffering agent that allows to maintain the pH of the buffer solution 2 pH units below the isoelectric point of the protein.

It has been shown by the inventors that histidine is particularly suited as a buffering agent when forming reversible protein complexes comprising different types of antibodies. The conjugate acid (protonated form) of the imidazole side chain in histidine has a pKα of approximately 6.0. Histidine buffers are most effective in a pH range from 5.5 to 7.4. Thus, histidine is particularly suited as a buffering agent when forming reversible protein complexes of antibodies with an isoelectric point between 6 and 9. Accordingly, in a particular embodiment, the invention relates to the method according to the invention, wherein the buffer solution comprises histidine as a buffering agent.

Further, it has been demonstrated that citrate is particularly suited as a buffering agent when forming reversible protein complexes comprising different types of antibodies. Citrate is a weak tricarboxylic acid with three different pKα values (3.1, 4.7, and 6.4). Citrate buffers are most effective in a pH range from 2.5 to 7. Thus, citrate is particularly suited as a buffering agent when forming reversible protein complexes of antibodies with an isoelectric point between 3 and 6, but may also be used for complexing protein with a higher isoelectric point. Accordingly, in a particular embodiment, the invention relates to the method according to the invention, wherein the buffer solution comprises citrate as a buffering agent.

The term, “buffering agent,” as used herein, refers to a weak acid or base used to maintain the pH of a solution near a chosen pH value after the addition of another acidic or basic compound. The function of such an agent is to prevent the change in pH when acids or bases are added to a solution. Such agents may be acids, bases, or combinations thereof.

In a particular embodiment, the invention relates to the method according to the invention, wherein the buffer solution comprising the protein and the complexing agent is obtained by mixing a first solution comprising the protein and a second solution comprising the complexing agent.

Within the present invention, it is preferred that the protein and the complexing agent are contacted by mixing a first solution comprising the protein with a second solution comprising the complexing agent. The skilled person is aware of methods for preparing solutions comprising proteins or complexing agents and to mix these solutions.

Alternatively, the protein and the complexing agent may be contacting by (i) providing either the protein or the complexing agent in a buffer solution and (ii) adding the remaining component of the RPC to the buffer solution in solid form.

In a particular embodiment, the invention relates to the method according to the invention, wherein the first solution comprising the protein and/or the second solution comprising the complexing agent comprises a buffering agent.

Both the solution comprising the protein and/or the solution comprising the complexing agent may comprise a buffering agent. That is, in certain embodiments, the method involves a step of contacting a first solution comprising a protein and a buffering agent with a second solution comprising a complexing agent and a buffering agent. The buffering agent in the first and second solution may be identical or may be a different buffering agent. In certain embodiments, both the first and second solution comprise histidine as the buffering agent. In other embodiments, both the first and second solution comprise citrate as the buffering agent.

Alternatively, the protein and the complexing agent may be contacted by mixing a solution comprising a buffering agent with a solid. For example, a solution comprising a protein and a buffering agent may be contacted with a complexing agent by adding the complexing agent in solid form to the solution such that the complexing agent dissolves in the solution and forms a reversible protein complex with the protein. Correspondingly, a solution comprising a complexing agent and a buffering agent may be contacted with a protein by adding the protein in solid form to the solution such that the protein dissolves in the solution and forms a reversible protein complex with the complexing agent.

In certain embodiments, the complexing agent is added gradually to a protein solution. Preferably, a solution comprising the complexing agent is added gradually to a protein solution. More preferably, a buffered solution comprising the complexing agent is added gradually to a buffered protein solution. The buffered solution may comprise any of the buffering agents disclosed herein.

Complex formation is driven by electrostatic interactions between oppositely charged proteins and complexing agents. Within the present invention, the protein and the complexing agent will be contacted in the buffer solution at a specific mole-charge ratio. In certain embodiments, the protein has a positive net charge when comprised in the buffer solution. In these cases, the mole-charge ratio between the protein and the complexing agent is defined as the ratio between the total number of positive charges on all proteins comprised in the buffer solution and the total number of negative charges on all complexing agents comprised in the buffer solution. Thus, at a mole-charge ratio of 1:1, the positive charges of the proteins comprised in the buffer solution should theoretically completely be neutralized by the negative charges of the complexing agents comprised in the buffer solution.

Within the present invention, it has been surprisingly found that a mole-charge ratio between the protein and the complexing agent of 1:1 or even higher than 1:1 (excess of protein) is sufficient for obtaining nearly complete complex formation, thereby reducing the demand for the complexing agent when producing the RPCs of the invention (Table 7). For example, it has been shown that for the complexing agent dextran sulfate a mole-charge ratio of 1:0.6 is sufficient to achieve complete complexation of proteins. For the complexing agent chondroitin sulfate, even a mole-charge ratio of 1:0.2 was sufficient to achieve complete complexation of proteins. Thus, in a particular embodiment, the invention relates to the method according to the invention, wherein the protein and the complexing agent are contacted at a mole-charge ratio ranging from 1:0.1 to 1:6, in particular wherein the protein and the complexing agent are contacted at a mole-charge ratio ranging from 1:0.2 to 1:1.

It is to be understood that the mole-charge ratio that is required to achieve complete complexation of the protein varies between complexing agents.

Thus, in certain embodiments, the invention relates to the method according to the invention, wherein the protein and the complexing agent dextran sulfate are contacted at a mole-charge ratio ranging from 1:0.2 to 1:2, preferably wherein the protein and the complexing agent dextran sulfate are contacted at a mole-charge ratio ranging from 1:0.5 to 1:2, more preferably wherein the protein and the complexing agent dextran sulfate are contacted at a mole-charge ratio ranging from 1:0.5 to 1:1, most preferably wherein the protein and the complexing agent dextran sulfate are contacted at a mole-charge ratio of 1:0.6.

In other embodiments, the invention relates to the method according to the invention, wherein the protein and the complexing agent chondroitin sulfate are contacted at a mole-charge ratio ranging from 1:0.2 to 1:2, preferably wherein the protein and the complexing agent chondroitin sulfate are contacted at a mole-charge ratio ranging from 1:0.2 to 1:1.

It further has been shown by the inventors that the complexing efficiency depends on the concentration of the protein in the buffer solution. In particular, it has been shown that complex formation can be achieved at protein concentrations ranging from 1-40 mg/mL (FIG. 10). Thus, in a particular embodiment, the invention relates to the method according to the invention, wherein the protein is contacted with the complexing agent in the buffer solution at a protein concentration ranging from 1-40 mg/mL.

It has been shown by the inventors that complexes formed at protein concentrations above 5 mg/mL have a larger particle size, which may have reduced the injectability of the complexes (FIG. 11). Further, complexes that have been formed at protein concentrations above 5 mg/mL do not dissociate as efficiently as protein complexes that have been formed at lower protein concentrations (FIG. 10). Thus, in a preferred embodiment, the invention relates to the method according to the invention, wherein the protein is contacted with the complexing agent in the buffer solution at a protein concentration ranging from 1-5 mg/mL.

The method of the present invention is not restricted to a specific type of protein. That is, the method of the present invention may be used for the production of reversible protein complexes comprising any type of protein. Since the skilled person is aware of methods to determine the isoelectric point of any protein based on its amino acid sequence, the skilled person is able to select buffer conditions that are suitable for the formation of reversible protein complexes with dextran sulfate or chondroitin sulfate.

Preferably, the method of the present invention is used for the production of reversible protein complexes comprising therapeutic proteins. That is, the method of the invention allows the production of pharmaceutical compositions with high protein concentrations that are suitable for subcutaneous, intramuscular or transdermal application.

The term “therapeutic protein,” as used herein, refers to any peptide or protein that is known to be useful for the prevention, treatment, or amelioration of a disease or disorder, e.g., an antibody, growth factor, cell surface receptor, cytokine, hormone, toxin, or fragments and/or fusion proteins of any of the foregoing. Thus, in a particular embodiment, the invention relates to the method according to the invention, wherein the protein is an antibody, a growth factor, a hormone, a cytokine, an enzyme, or a fragment and/or fusion protein of any of the foregoing.

The present invention is directed to the production of reversible protein complexes. However, it is to be noted that the present invention also encompasses the production of reversible complexes comprising peptides. Thus, the term protein is used herein interchangeably with the term peptide or polypeptide.

The term “peptide” or “polypeptide” as used herein refers to a compound made up of a single unbranched chain of amino acid residues linked by peptide bonds. The number of amino acid residues in such compounds varies widely.

The term “protein” as used herein may be used synonymously with the term “ peptide” or “polypeptide” or may refer to, in addition, a complex of two or more peptides which may be linked by bonds other than peptide bonds, for example, such peptides making up the protein may be linked by disulfide bonds. The term “protein” may also comprehend peptide(s) or a family of peptides having identical amino acid sequence(s) but different post-translational modification(s), such as phosphorylation(s), acylation(s), glycosylation(s), and the like, particularly as may be added when such proteins are expressed in eukaryotic hosts.

Examples of proteins encompassed within the definition herein include mammalian proteins, such as, e.g., growth hormone, including human growth hormone and bovine growth hormone; growth hormone releasing factor; parathyroid hormone; thyroid stimulating hormone; lipoproteins; α-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or tissue-type plasminogen activator (t-PA, e.g., Activase®, TNKase®, Retevase®); bombazine; thrombin; tumor necrosis factor-α and -β; enkephalinase; RANTES (regulated on activation normally T-cell expressed and secreted); human macrophage inflammatory protein (MIP-1-α); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins; CD proteins such as CD3, CD4, CD8, CD19 and CD20; erythropoietin (EPO); thrombopoietin (TPO); osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides.

The term “growth factor”, as used herein, refers to a polypeptide molecule that is capable of effectuating differentiation of cells. Examples of growth factors as contemplated for use in accord with the teachings herein include an epidermal growth factor (EGF), transforming growth factor-alpha (TGF-alpha), transforming growth factor-beta (TGF-beta), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), and/or platelet derived growth factor (PDGF).

The term “hormone” as used herein refers to a substance normally formed by one organ that stimulates the function of another organ. Polypeptide hormones include, but are not limited to, insulin, growth hormone, gastric inhibitory polypeptide, and cholecystokinin.

The term “cytokine,” as used herein, means any secreted polypeptide that affects the functions of other cells, and that modulates interactions between cells in the immune or inflammatory response. Cytokines include, but are not limited to monokines, lymphokines, and chemokines regardless of which cells produce them. For instance, a monokine is generally referred to as being produced and secreted by a monocyte, however, many other cells produce monokines, such as natural killer cells, fibroblasts, basophils, neutrophils, endothelial cells, brain astrocytes, bone marrow stromal cells, epidermal keratinocytes, and B-lymphocytes. Lymphokines are generally referred to as being produced by lymphocyte cells. Examples of cytokines include, but are not limited to, interleukin-1 (IL-1), interleukin-6 (IL-6), Tumor Necrosis Factor alpha (TNFα), and Tumor Necrosis Factor beta (TNFβ).

The term “enzyme” as used herein refers to a macromolecular compound mainly comprised of a protein and catalyzing a chemical reaction.

Preferably, the method of the present invention is used for the production of reversible protein complexes comprising antibodies. Thus, in a preferred embodiment, the invention relates to the method according to the invention, wherein the protein is an antibody, or a fragment thereof.

In a particular embodiment, the invention relates to the method according to the invention, wherein the antibody is a monoclonal antibody, a polyclonal antibody, a chimeric antibody, a multispecific antibody, an antibody fusion protein, an antibody-drug-conjugate or an antibody fragment.

The term “antibody” herein is used in the broadest sense and specifically covers monoclonal antibodies, polyclonal antibodies, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs (complementary determining regions) on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one embodiment, however, the immunoglobulin is of human, murine, or rabbit origin.

The antibody may be an intact antibody. The term “intact antibody” as used herein is one comprising a VL and VH domains, as well as a light chain constant domain (CL) and heavy chain constant domains, CH1, CH2 and CH3. The constant domains may be native sequence constant domains (e.g., human native sequence constant domains) or amino acid sequence variant thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell- mediated cytotoxicity (ADCC); phagocytosis; and down regulation of cell surface receptors such as B cell receptor and BCR.

The term “Fc region” as used herein means a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “framework” or “FR” as used herein refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2- H2(L2)-FR3-H3(L3)-FR4.

The terms “full length antibody”, “intact antibody,” and “whole antibody” are used herein interchangeably to refer to an antibody having a structure substantially similar to a native antibody structure or having heavy chains that contain an Fc region as defined herein.

The term “antibody fragment(s)” as used herein comprises a portion of a full length antibody, generally the antigen binding or variable region thereof. Examples of antibody fragments include Fab, Fab′, F(ab′)2, and Fv fragments; diabodies; linear antibodies; minibodies (Olafsen et al (2004) Protein Eng. Design & Sel.17(4):315-323), fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

In certain embodiments, the antibody may be a monoclonal antibody. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the subject matter described herein may be made by the hybridoma method first described by Kohler et al (1975) Nature, 256:495, or may be made by recombinant DNA methods (see for example: US 4816567; US 5807715). The monoclonal antibodies may also be isolated from phage antibody libraries using the techniques described in Clackson et al (1991) Nature, 352:624- 628; Marks et al (1991) J. Mol. Biol., 222:581-597; for example.

In certain embodiments, the antibody may be a native antibody. “Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N— to C-terminus, each heavy chain has a variable region (VH), also called a variable heavy domain or a heavy chain variable domain, followed by three constant domains (CH1, CH2, and CH3). Similarly, from N— to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

In certain embodiments, the antibody is an engineered antibody. An “engineered antibody” may be any antibody in which one or more amino acid residues have been introduced, deleted or substituted by means of genetic engineering. The term “engineered antibody” further encompasses “glycoengineered antibodies”. “Glycoengineereid antibodies” are antibodies in which the composition of the attached glycans has been modified. Modification of the glycans may be achieved, without limitation, chemically or enzymatically. Further, genetically modified host cells are known in the art that may be used for the synthesis of glycoengineered antibodies.

In certain embodiments, the antibody is a human antibody. A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (US 4816567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape, etc.) and human constant region sequences.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

In other embodiments, the antibody is a polyclonal antibody. The term “polyclonal antibody” as used herein refers to a heterogeneous mixture of antibodies that recognize and bind to different epitopes on the same antigen. Polyclonal antibodies may be obtained from crude serum preparations or may be purified using, for example, antigen affinity chromatography, Protein A/Protein G affinity chromatography, and the like.

In certain embodiments, an antibody provided herein is a multispecific antibody, e.g. a bispecific antibody. The term “multispecific antibody” as used herein refers to an antibody comprising an antigen-binding domain that has polyepitopic specificity (i.e., is capable of binding to two, or more, different epitopes on one molecule or is capable of binding to epitopes on two, or more, different molecules).

In some embodiments, multispecific antibodies are monoclonal antibodies that have binding specificities for at least two different antigen binding sites (such as a bispecific antibody). In some embodiments, the first antigen-binding domain and the second antigen-binding domain of the multispecific antibody may bind the two epitopes within one and the same molecule (intramolecular binding). For example, the first antigen-binding domain and the second antigen-binding domain of the multispecific antibody may bind to two different epitopes on the same protein molecule. In certain embodiments, the two different epitopes that a multispecific antibody binds are epitopes that are not normally bound at the same time by one monospecific antibody, such as e.g. a conventional antibody or one immunoglobulin single variable domain. In some embodiments, the first antigen-binding domain and the second antigen-binding domain of the multispecific antibody may bind epitopes located within two distinct molecules (intermolecular binding). For example, the first antigen-binding domain of the multispecific antibody may bind to one epitope on one protein molecule, whereas the second antigen-binding domain of the multispecific antibody may bind to another epitope on a different protein molecule, thereby cross-linking the two molecules.

In some embodiments, the antigen-binding domain of a multispecific antibody (such as a bispecific antibody) comprises two VH/VL units, wherein a first VH/VL unit binds to a first epitope and a second VH/VL unit binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, and antibody fragments (such as Fab, Fv, dsFv, scFv, diabodies, bispecific diabodies and triabodies, antibody fragments that have been linked covalently or non-covalently). A VH/VL unit that further comprises at least a portion of a heavy chain variable region and/or at least a portion of a light chain variable region may also be referred to as an “arm” or “hemimer” or “half antibody.” In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation.

In certain embodiments, a multispecific antibody provided herein may be a bispecific antibody. The term “bispecific antibody” as used herein refers to a multispecific antibody comprising an antigen-binding domain that is capable of binding to two different epitopes on one molecule or is capable of binding to epitopes on two different molecules. A bispecific antibody may also be referred to herein as having “dual specificity” or as being “dual specific.” Exemplary bispecific antibodies may bind both protein and any other antigen. In certain embodiments, bispecific antibodies may bind to two different epitopes of the same protein molecule. In certain embodiments, bispecific antibodies may bind to two different epitopes on two different protein molecules. Bispecific antibodies may also be used to localize cytotoxic agents to cells which express protein.

Bispecific antibodies can be prepared as full-length antibodies or antibody fragments. In certain embodiments, the bispecific antibody fragment is a dutafab, as disclosed by Beckmann et al. (DutaFabs are engineered therapeutic Fab fragments that can bind two targets simultaneously; Nature Communications; 2021; vol.2:708). In certain embodiments, the dutafab may specifically bind to human vascular endothelial growth factor (VEGF/VEGF-A) and human angiopoietin-2 (Ang-2). In certain embodiments, the dutafab may specifically bind to human vascular endothelial growth factor (VEGF/VEGF-A) and to a human platelet-derived growth factor (PDGF).

Engineered antibodies with three or more functional antigen binding sites, including “Octopus antibodies” or“dual-variable domain immunoglobulins” (DVDs) are also included herein (see, e.g., US 2006/0025576A1, and Wu et al. Nature Biotechnology (2007)).). The antibody or fragment herein also includes a″Dual Acting FAb” or“DAF” comprising an antigen binding site that binds to a target protein as well as another, different antigen (see, US 2008/0069820, for example).

The antibody of the present invention may be a naked antibody, a fusion antibody or comprised in an antibody-drug conjugate.

A “naked antibody” refers to an antibody that is not conjugated to a heterologous moiety (e.g., a cytotoxic moiety) or radiolabel.

The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate.

By the term “antibody fusion protein” as used herein, is meant a polypeptide molecule having an amino acid sequence which comprises the amino acid sequence of a portion of an antigen-binding protein. The portion of the antigen-binding protein may, for example, be an entire antibody or a fragment thereof. In particular, the antibody fusion protein may comprise a first amino acid sequence of an antibody or a fragment thereof, and a second amino acid sequence of another polypeptide or protein. The second amino acid sequence may for example be an amino acid sequence of a cytokine. The antibody fusion protein may be created through the joining of two or more polynucleotides which originally coded for separate proteins (including peptides and polypeptides). Translation of the fusion gene results in a single protein with functional properties derived from each of the original proteins.

In certain embodiments, the protein is an antibody. Exemplary molecular targets for antibodies encompassed by the present invention include CD proteins such as CD3, CD4, CD8, CD19, CD20 and CD34; members of the HER receptor family such as the EGF receptor, HER2, HER3 or HER4 receptor; cell adhesion molecules such as LFA-1, Mol, p150,95, VLA-4, ICAM-1, VCAM and αv/β3 integrin including either α or β subunits thereof (e.g. anti-CD11a, anti-CD18 or anti-CD11b antibodies); growth factors such as VEGF; IgE; blood group antigens; flk2/flt3 receptor; obesity (OB) receptor; protein C etc.

Exemplary antibodies that may be utilized include, but are not limited to, hRl (anti- IGF-1R, U.S. Pat. Application Serial No. 12/722,645, filed 3/12/10), hPAM4 (anti-mucin, U.S. Pat. No. 7,282,567), hA20 (anti-CD20, U.S. Pat. No. 7,251 ,164), hA19 (anti-CD19, U.S. Pat. No. 7,109,304), WMMU31 (anti-AFP, U.S. Pat. No. 7,300,655), hLLl (anti- CD74, U.S. Pat. No. 7,312,318), hLL2 (anti-CD22, U.S. Pat. No. 7,074,403), hMu-9 (anti-CSAp, U.S. Pat. No. 7,387,773), hL243 (anti-HLA-DR, U.S. Pat. No. 7,612,180), hMN-14 (anti-CEACAM5, U.S. Pat. No. 6,676,924), hMN-15 (anti-CEACAM6, U.S. Pat. No. 7,541 ,440), hRS7 (anti-EGP-1 , U.S. Pat. No. 7,238,785), hMN-3 (anti- CEACAM6, U.S. Pat. No. 7,541,440), Ab124 and Abl 25 (anti-CXCR4, U.S. Pat. No. 7, 138,496).

Alternative antibodies of use include, but are not limited to, abciximab (anti- glycoprotein Ilb/IIIa), alemtuzumab (anti-CD52), bevacizumab (anti-VEGF), cetuximab (anti-EGFR), gemtuzumab (anti-CD33), ibritumomab tiuxetan (anti-CD20), panitumumab (anti-EGFR), rituximab (anti-CD20), tositumomab (anti-CD20), trastuzumab (anti-ErbB2), abagovomab (anti-CA-125), adecatumumab (anti-EpCAM), atlizumab (anti-IL-6 receptor), benralizumab (anti-CD125), CC49 (anti-TAG-72), AB-PG1 -XG1-026 (anti-PSMA, U.S. Patent Application 11/983,372, deposited as ATCC PTA-4405 and PTA-4406), D2/B (anti- PSMA, WO 2009/130575), tocilizumab (anti-IL-6 receptor), basiliximab (anti-CD25), daclizumab (anti-CD25), efalizumab (anti-CD11a), GA101 (anti-CD20; Glycart Roche), muromonab-CD3 (anti-CD3 receptor), natalizumab (anti-a4 integrin), omalizumab (anti- IgE); anti-TNF- a antibodies such as CDP571 (Ofei et al., 2011 , Diabetes 45:881 -85), MTNFAI, M2TNFAI, M3TNFAI, M3TNFABI, M302B, M303 (Thermo Scientific, Rockford, IL), infliximab (Centocor, Malvern, PA), certolizumab pegol (UCB, Brussels, Belgium), anti-CD40L (UCB, Brussels, Belgium), adalimumab (Abbott, Abbott Park, IL), Benlysta (Human Genome Sciences); antibodies for therapy of Alzheimer’s disease such as Alz 50 (Ksiezak-Reding et al., 1987, J Biol Chem 263:7943-47), gantenerumab, solanezumab and infliximab; anti-fibrin antibodies like 59D8, T2G1 s, MH1 ; anti-HIV antibodies such as P4/D10 (U.S. Pat. Application Serial No. 11/745,692), Ab 75, Ab 76, Ab 77 (Paulik et al., 1999, Biochem Pharmacol 58: 1781-90); and antibodies against pathogens such as CR6261 (anti-influenza), exbivirumab (anti-hepatitis B), felvizumab (anti-respiratory syncytial virus), foravirumab (anti-rabies virus), motavizumab (anti-respiratory syncytial virus), palivizumab (anti-respiratory syncytial virus), panobacumab (anti-Pseudomonas), rafivirumab (anti-rabies virus), regavirumab (anti-cytomegalovirus), sevirumab (anti-cytomegalovirus), tivirumab (anti-hepatitis B), and urtoxazumab (anti-E. coli).

In certain embodiments, the antibody may be a bispecific anti-VEGF/anti-angiopoietin-2 (Ang-2) antibody, an anti-alpha synuclein (aSyn) antibody, a bispecific anti-FAP/anti-OX40 antibody, a bispecific anti-VEGF/anti-PDGF antibody (dutafab), Bevacizumab, Pertuzumab or Gantenerumab.

That is, in a certain embodiment, the antibody may be a bispecific anti-VEGF/anti-angiopoietin-2 (Ang-2) antibody. In a certain embodiment, the bispecific anti-VEGF/anti-angiopoietin-2 (Ang-2) antibody may be faricimab, as disclosed in WO2014/009465 as “VEGFang2-0016”. In a certain embodiment, the bispecific anti-VEGF/anti-angiopoietin-2 (Ang-2) antibody may be a dutafab.

In a certain embodiment, the antibody may be an anti-alpha synuclein (aSyn) antibody. In a certain embodiment, the antibody may be a bispecific anti-FAP/anti-OX40 antibody. In a certain embodiment, the antibody may be a dutafab. In a certain embodiment, the dutafab may be a bispecific anti-VEGF/anti-PDGF dutafab. In a certain embodiment, the antibody may be Bevacizumab. In a certain embodiment, the antibody may be Pertuzumab. In a certain embodiment, the antibody may be Gantenerumab.

In another embodiment, the antibody is human or humanized. In one aspect, the antibody is selected from alemtuzumab (LEMTRADA®), atezolizumab (TECENTRIQ®), bevacizumab (AVASTIN®), cetuximab (ERBITUX®), panitumumab (VECTIBIX®), pertuzumab (OMNITARG®, 2C4), trastuzumab (HERCEPTIN®), tositumomab (Bexxar®), abciximab (REOPRO®), adalimumab (HUMIRA®), apolizumab, aselizumab, atlizumab, bapineuzumab, basiliximab (SIMULECT®), bavituximab, belimumab (BENLYSTA®) briankinumab, canakinumab (ILARIS®), cedelizumab, certolizumab pegol (CIMZIA®), cidfusituzumab, cidtuzumab, cixutumumab, clazakizumab, crenezumab, daclizumab (ZENAPAX®), dalotuzumab, denosumab (PROLIA®, XGEVA®), eculizumab (SOLIRIS®), efalizumab, epratuzumab, erlizumab, emicizumab (HEMLIBRA®), felvizumab, fontolizumab, golimumab (SIMPONI®), ipilimumab, imgatuzumab, infliximab (REMICADE®), labetuzumab, lebrikizumab, lexatumumab, lintuzumab, lucatumumab, lulizumab pegol, lumretuzumab, mapatumumab, matuzumab, mepolizumab, mogamulizumab, motavizumab, motovizumab, muronomab, natalizumab (TYSABRI®), necitumumab (PORTRAZZA®), nimotuzumab (THERACIM®), nolovizumab, numavizumab, olokizumab, omalizumab (XOLAIR®), onartuzumab (also known as MetMAb), palivizumab (SYNAGIS®), pascolizumab, pecfusituzumab, pectuzumab, pembrolizumab (KEYTRUDA®), pexelizumab, priliximab, ralivizumab, ranibizumab (LUCENTIS®), reslivizumab, reslizumab, resyvizumab, robatumumab, rontalizumab, rovelizumab, ruplizumab, sarilumab, secukinumab, seribantumab, sifalimumab, sibrotuzumab, siltuximab (SYLVANT®) siplizumab, sontuzumab, tadocizumab, talizumab, tefibazumab, tocilizumab (ACTEMRA®), toralizumab, tucusituzumab, umavizumab, urtoxazumab, ustekinumab (STELARA®), vedolizumab (ENTYVIO®), visilizumab, zanolimumab, zalutumumab.

In a particular embodiment, the invention relates to the method according to the invention, wherein the complexing agent has a negative net charge when comprised in the buffer solution.

Within the present invention, it is preferred that the pH of the buffer solution is adjusted to be lower than the isoelectric point of the protein. At the same time, it is preferred that the complexing agent has a negative charge when comprised in the buffer solution. The complexing agents dextran sulfate and chondroitin sulfate comprise a sulfate group which may be negatively charged when comprised in the buffer solution. In particular, the sulfate group will be negatively charged when the pH of the buffer solution is adjusted to a pH value that is higher than the pKα value of the sulfate group of dextran sulfate and/or chondroitin sulfate.

Thus, in a particular embodiment, the invention relates to the method according to the invention, wherein the pH of the buffer solution is adjusted to be lower than the isoelectric point of the protein and higher than the pKα value of the buffering agent, in particular wherein the buffering agent is dextran sulfate and/or chondoritin sulfate.

In a particular embodiment, the invention relates to the method according to the invention, wherein the composition comprising the RPCs comprises at least one excipient.

It has been shown by the inventors that the presence of excipients in the buffering solution does not significantly interfere with the formation of reversible protein complexes or the dissolution of these complexes. For example, it has been shown that the commonly used excipients sucrose, polysorbate 20 and poloxamer 188 either alone or in combination do not interfere with reversible protein complex formation and dissolution (FIGS. 14 and 15). Thus, the excipient(s) may be added to the buffer solution before the formation of the RPCs, such that the RPCs form in the presence of the excipient(s). Accordingly, in a particular embodiment, the invention relates to the method according to the invention, wherein the at least one excipient is added to the composition before the formation of the RPCs.

Alternatively, the excipient(s) may be added to the suspension after the formation of the RPCs. That is, the RPCs may be formed in the absence of any excipient and the excipients are added to the suspensions subsequently. Thus, in another embodiment, the invention relates to the method according to the invention, wherein the at least one excipient is added to the composition after the formation of the RPCs.

In further embodiments, excipient(s) may be added to the composition of the invention before and after formation of the RPCs.

In a particular embodiment, the invention relates to the method according to the invention, wherein the at least one excipient is a stabilizer and/or a surfactant.

The term “stabilizer”, as used herein, denotes a pharmaceutically acceptable excipient, which protects the protein and/or the composition from chemical and/or physical degradation during manufacturing, storage and application. Stabilizers include but are not limited to saccharides, amino acids, polyols, e.g. mannitol, sorbitol, xylitol, dextran, glycerol, arabitol, propylene glycol, polyethylene glycol, cyclodextrines, e.g. hydroxypropyl-β-cyclodextrine, sulfobutylethyl-β-cyclodextrine, β-cyclodextrine, polyethylenglycols, e.g. PEG 3000, PEG 3350, PEG 4000, PEG 6000, albumines, e.g. human serum albumin (HSA), bovine serum albumin (BSA), salts, e.g. sodium chloride, magnesium chloride, calcium chloride, chelators, e.g. EDTA as hereafter defined. More than one stabilizer, selected from the same or from different groups, can be present in the composition.

The term “saccharide” as used herein includes monosaccharides and oligosaccharides. A monosaccharide is a monomeric carbohydrate which is not hydrolysable by acids, including simple sugars and their derivatives, e.g. aminosugars. Saccharides are usually in their D conformation. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a linear chain. The monomeric saccharide units within an oligosaccharide can be identical or different. Depending on the number of monomeric saccharide units the oligosaccharide is a di-, tri-, tetra- penta- and so forth saccharide. In contrast to polysaccharides the monosaccharides and oligosaccharides are water soluble. Examples of oligosaccharides include sucrose, trehalose, lactose, maltose and raffinose. Preferred saccharides are sucrose and trehalose (i.e. α,α—D—trehalose), most preferred is sucrose.

The term “amino acid” as used herein denotes a pharmaceutically acceptable organic molecule possessing an amino moiety located at α-position to a carboxylic group. Examples of amino acids include but are not limited to arginine, glycine, ornithine, lysine, histidine, glutamic acid, asparagic acid, isoleucine, leucine, alanine, phenylalanine, tyrosine, tryptophane, methionine, serine, proline. The amino acid employed is preferably in each case the L-form. Basic amino acids, such as arginine, histidine, or lysine, are preferably employed in the form of their inorganic salts (advantageously in the form of the hydrochloric acid salts, i.e. as amino acid hydrochlorides).

A subgroup within the stabilizers are lyoprotectants. The term “lyoprotectant” denotes pharmaceutically acceptable excipients, which protect the labile active ingredient (e.g. a protein) against destabilizing conditions during the lyophilisation process, subsequent storage and reconstitution. Lyoprotectants comprise but are not limited to the group consisting of saccharides, polyols (such as e.g. sugar alcohols) and amino acids. Preferred lyoprotectants can be selected from the group consisting of saccharides such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine or mixtures thereof.

A subgroup within the stabilizers are antioxidants. The term “antioxidant” denotes pharmaceutically acceptable excipients, which prevent oxidation of the active pharmaceutical ingredient. Antioxidants comprise but are not limited to ascorbic acid, gluthathione, cysteine, methionine, citric acid, EDTA.

The composition according to the invention may also comprise one or more tonicity agents. The term “tonicity agents” denotes pharmaceutically acceptable excipients used to modulate the tonicity of the composition. The composition may be hypotonic, isotonic or hypertonic. Isotonicity in general relates to the osmotic pressure of a solution, usually relative to that of human blood serum (around 250-350 mOsmol/kg). The composition according to the invention may be hypotonic, isotonic or hypertonic but will preferably be isotonic. An isotonic composition is liquid or liquid reconstituted from a solid form, e.g. from a lyophilized form, and denotes a solution having the same tonicity as some other solution with which it is compared, such as physiologic salt solution and the blood serum. Suitable tonicity agents comprise but are not limited to sodium chloride, potassium chloride, glycerin and any component from the group of amino acids or sugars, in particular glucose.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is sucrose.

The compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, e.g. paraben, chlorobutanol, phenol, sorbic acid, and the like. Preservatives comprise but are not limited to ethanol, benzyl alcohol, phenol, m-cresol, p-chlor-m-cresol, methyl or propyl parabens, benzalkonium chloride.

The term “surfactant” as used herein denotes a pharmaceutically acceptable, surface-active agent. Preferably, a non-ionic surfactant is used. Examples of pharmaceutically acceptable surfactants include, but are not limited to, polyoxyethylen-sorbitan fatty acid esters (Tween), polyoxy ethylene alkyl ethers (Brij), alkylphenylpolyoxyethylene ethers (Triton X), polyoxyethylene-polyoxypropylene copolymers (Poloxamer, Pluronic), and sodium dodecyl sulphate (SDS). Preferred polyoxyethylene-sorbitan fatty acid esters are polysorbate 20 (polyoxy ethylene sorbitan mono laureate, sold under the trademark Tween 20™) and polysorbate 80 (polyoxy ethylene sorbitan monooleate, sold under the trademark Tween 80™). Preferred polyethylene-polypropylene copolymers are those sold under the names Pluronic® F68 or Poloxamer 188™. Preferred polyoxyethylene alkyl ethers are those sold under the trademark Brij™. Preferred alkylphenylpolyoxyethylene ethers are sold under the tradename Triton X, most preferred is p-tert-octylphenoxy polyethoxyethanol (sold under the tradename Triton X- 100™).

It is to be understood that the stabilizers and surfactants listed above may be added to the compositions of the invention at a concentration that is sufficient to obtain the intended effect. The skilled person is aware of these concentrations or, alternatively, is able to determine these concentrations by routine experimentation.

A preferred stabilizer in the compositions of the present invention is sucrose. Sucrose is commonly used as a stabilizer in pharmaceutical compositions due to its protein-stabilizing properties. In certain embodiments, sucrose is added to the composition of the invention before the RPCs are formed. That is, sucrose is added to the buffer solution before the protein and the complexing agent are contacted in said buffer solution. In certain embodiments, sucrose is present in the buffer solution at a concentration ranging from 50 to 500 mM, preferably ranging from 100 to 250 mM.

In other embodiments, sucrose may be added to the composition after the formation of the RPCs. That is, in certain embodiments, sucrose may be added to a suspension before a spray drying or lyophilization step. In certain embodiments, sucrose may be added to a suspension according to the invention at a concentration ranging from 0.5 to 10 mg/mL, ranging from 0.5 to 5 mg/mL, or ranging from 1 to 3 mg/mL.

Preferred solubilizers in the compositions of the present invention are poloxamer 188 and polysorbate 20. Poloxamer 188 and polysorbate 20 are commonly used as solubilizers in pharmaceutical compositions. In certain embodiments, poloxamer 188 and/or polysorbate 20 are added to the composition of the invention before the formation of RPCs. That is, poloxamer 188 and/or polysorbate 20 may be added to the buffer solution before the contacting of the protein and the complexing agent in said buffer solution. In certain embodiments, poloxamer 188 and/or polysorbate 20 are present in the buffer solution at a concentration ranging from 0.01 to 1% (w/v), ranging from 0.01 to 0.5% (w/v), ranging from 0.01 to 0.1% (w/v), or ranging from 0.02 to 0.06% (w/v).

In other embodiments, poloxamer 188 and/or polysorbate 20 may be added to the composition, in particular any suspension of the present invention, after the formation of the RPCs. That is, in certain embodiments, poloxamer 188 and/or polysorbate 20 may be added to a composition comprising RPCs or an enriched RPC suspension before the spray drying step. In certain embodiments, poloxamer 188 and/or polysorbate 20 may be added to a suspension according to the invention at a concentration ranging from 0.1 to 2 mg/mL, ranging from 0.1 to 1 mg/mL, or ranging from 1 to 0.5 mg/mL.

In certain embodiments, the suspension comprising RPCs or the enriched RPC suspension is free of stabilizers and/or solubilizers. That is, in certain embodiments, the suspension comprising RPCs or the enriched RPC suspension comprises one or more stabilizer but is free of solubilizers. In other embodiments, the suspension comprising RPCs or the enriched RPC suspension comprises one or more solubilizers but is free of stabilizers. In further embodiments, the suspension comprising RPCs or the enriched RPC suspension is free of solubilizers and stabilizers.

In a particular embodiment, the invention relates to the method according to the invention, wherein the method comprises a further step of exchanging the liquid fraction of the suspension comprising the RPCs.

That is, the liquid fraction of the suspension comprising the RPCs may be replaced with another liquid. The skilled person is aware of methods for exchanging the liquid fraction of a suspension.

For example, the liquid fraction of the suspension comprising the RPCs may be exchanged by centrifugation. That is, the suspension comprising the RPCs may be centrifuged at an appropriate speed to facilitate sedimentation of the RPCs. Subsequently, the sedimented RPCs may be resuspended in another liquid. The centrifugation and resuspension steps may be repeated 1, 2, 3, 4, 5 or more times. Preferably, the sedimented RPCs are resuspended in a buffer solution or in water.

Thus, in a particular embodiment, the invention relates to the method according to the invention, wherein the liquid fraction of the suspension comprising the RPCs is exchanged by centrifugation of the suspension comprising the RPCs and resuspension of the sedimented RPCs in a buffer solution or water.

Alternatively, the liquid fraction of the suspension comprising the RPCs may be exchanged by dialysis. Dialysis may be performed, without limitation, in a dialysis cartridge or in a dialysis tube. The skilled person is able to select a suitable molecular weight cutoff of the dialysis cartridge or the dialysis tube based on the size of the protein comprised in the RPC, such that the RPCs are retained in the dialysis cartridge or tube. Preferably, the dialysis cartridge or tube may have a molecular weight cutoff ranging from 10 to 10,000 kDa. Dialysis of the RPCs may be performed against any liquid, preferably against a buffer solution or water. The dialysis step may be repeated 1, 2, 3, 4 or 5 times.

Thus, in a particular embodiment, the invention relates to the method according to the invention, wherein the liquid fraction of the suspension comprising the RPCs is exchanged by dialysis of the suspension comprising the RPCs against a buffer solution or water.

In certain embodiments, the liquid fraction of the suspension comprising the RPCs is exchanged with a buffer solution. In certain embodiments, the buffer solution may have a similar or identical composition as the buffer solution in which the protein and/or the complexing agent have been dissolved before the formation of the RPCs. That is, in certain embodiments, the liquid fraction of the suspension comprising the RPCs may be exchanged with a fresh buffer solution. A fresh buffer solution is a buffer solution which does not comprise a protein, a complexing agent and/or an RPC. In certain embodiments, the fresh buffer solution may be any of the buffer solutions disclosed herein. In certain embodiments, the liquid fraction of the suspension comprising the RPCs may be exchanged with 20 mM histidine buffer (pH 5).

In certain embodiments, the liquid fraction of the suspension comprising the RPCs is exchanged with water. It has been surprisingly shown by the inventors that dialysis of RPCs against ultrapure water results in very small particles with diameters in the nanometer range. Such small RPCs are particularly attractive for the administration to patients through a syringe. That is, in one preferred embodiment, RPCs are dialyzed against ultrapure water.

The term “water” refers to the chemical compound having the chemical formula H₂O. Within the meaning of the present invention, water is free or substantially free of solutes. Preferably, the water used in the method of the invention is distilled water and/or deionized water.

In certain embodiments, the water used in the method of the invention is ultrapure water. The term “ultrapure water” as used herein means water from which impurities have been removed as much as possible and which has a specific resistance of 16 MΩ·cm or above. In certain embodiments, the term “ultrapure water” means water with a specific resistance of at least 17 MQ cm. In certain embodiments, the term “ultrapure water” means water with a specific resistance of at least 18 MQ cm. The term ultrapure water encompasses Ultra pure water (MilliQ water).

In a particular embodiment, the invention relates to the method according to the invention, wherein the method comprises a further step of enriching the RPCs in the suspension to obtain an enriched RPC suspension.

The method of the present invention may comprise a further step of enriching the RPCs in the suspension. That is, in a particular embodiment, the invention relates to the method according to the invention, wherein enriching the RPCs in the suspension comprises the steps of: (a) centrifuging the suspension comprising the RPCs to obtain a supernatant and a precipitate comprising an enriched RPC suspension; and (b) removing the supernatant from the precipitate to obtain an enriched RPC suspension.

The RPCs in the suspension may be enriched by any method known in the art. Preferably, the RPCs in the suspension are enriched by centrifugation. The RPCs in the suspension have a higher density than the soluble components of the suspension. Thus, centrifugation of the suspension will result in the migration of the RPCs towards the bottom of the centrifugation vessel. Exhaustive centrifugation will consequently result in the formation of a precipitate or pellet comprising the RPCs at the bottom of the vessel and of a supernatant that is substantially free of RPCs. The supernatant is said to be “substantially free” of RPC if, after the centrifugation step, less than 10%, less than 9%, less than 8%, less than 7%, less than 6%, less than 5%, less than 4%, less than 3%, less than 2%, or less than 1% of the RPCs in a sample are comprised in the supernatant. The skilled person is aware that the concentration of RPCs in the supernatant depends on the duration and the speed of the centrifugation step.

To enrich the RPCs in the suspension, the supernatant may be partially or completely removed after the centrifugation step. That is, in certain embodiments, the supernatant is partially removed from the centrifugation vessel and the precipitate is subsequently re-suspended in the remaining supernatant to obtain an enriched suspension. In other embodiments, the supernatant may be removed completely from the centrifugation vessel and the precipitate may be re-suspended in a liquid that is added to the centrifugation vessel. In certain embodiments, this liquid has a lower volume than the supernatant that has been removed in the first step. The liquid in which the precipitate is resuspended may be identical to the buffer solution in which the RPCs have been formed or may be different from the buffer solution in which the RPCs have been formed.

The term “centrifugation” as used herein refers to the rotation in a compartment of an apparatus, said compartment spun about an axis for the purpose of separating materials. The term “precipitate”, as used herein, refers to any solid or semisolid material capable of being physically separated from the fluid portion of the suspension. The term “supernatant,” as used herein, describes the fluid portion of the suspension, after particles, such as RPCs, have settled to the bottom of the vessel.

Besides centrifugation, other methods may be used for obtaining an enriched suspension comprising RPCs. For example, in certain embodiments, an enriched suspension may be obtained by letting the RPCs in the suspension settle by gravity and decanting the supernatant. In other embodiments, an enriched suspension comprising RPCs may be obtained by filtration and/or dialysis of the suspension comprising RPCs.

The skilled person is aware of means of centrifugation to obtain enriched RPC suspensions. Further, the skilled person is aware that the concentration of RPCs in the enriched RPC suspensions depends at least on the centrifugation speed, the centrifugation time and the volume of the liquid the RPCs are re-suspended in. Thus, the skilled person is able to adjust the centrifugation method such that an enriched RPC suspension with a desired protein concentration can be obtained.

Within the present invention, the RPCs in the suspension may be enriched to obtain a protein concentration above 50 mg/mL. Preferably, the suspension comprising RPCs may be enriched to obtain a protein concentration between 50 and 300 mg/mL. More preferably, the suspension comprising RPCs may be enriched to obtain a protein concentration between 50 and 250 mg/mL. Most preferably, the suspension comprising RPCs may be enriched to obtain a protein concentration between 100 and 250 mg/mL.

It has to be noted that the enrichment step may be performed before or after exchanging the liquid fraction of the suspension comprising the RPCs. That is, in certain embodiments, the liquid fraction of the suspension comprising the RPCs may be exchanged first and the RPCs in the obtained suspension are enriched subsequently. For example, the liquid fraction of the suspension comprising the RPCs may be dialysed against ultrapure water in a first step and the RPCs in the resulting suspension may then be enriched to a desired concentration by centrifugation in a second step.

In certain embodiments, RPCs in a suspension may first be enriched to a desired concentration and the liquid fraction of the enriched suspension may then be exchanged by centrifugation and resuspension or by dialysis in a second step.

In certain embodiments, RPCs may be enriched while the liquid fraction of the suspension comprising the RPCs is exchanged. That is, RPCs may be sedimented by centrifugation and subsequently resuspended in a smaller volume of a fresh buffer solution or ultrapure water. Optionally, the RPCs may be washed one or multiple times before the final resuspension in a smaller volume.

In a particular embodiment, the invention relates to the method according to the invention, wherein the liquid fraction of the enriched RPC suspension is at least in part replaced with a non-aqueous solvent during the enrichment step.

After the centrifugation step, the supernatant may be partially or completely removed from the centrifugation vessel and replaced with a non-aqueous solvent. That is, in certain embodiments, the supernatant may be removed completely after the centrifugation step and the precipitate comprising the RPCs may be resuspended in a non-aqueous solvent. In other embodiments, the supernatant may be removed partially after the centrifugation step and the precipitate may be resuspended in a mixture of the remaining supernatant and a non-aqueous solvent.

In a particular embodiment, the invention relates to the method according to the invention, wherein the non-aqueous solvent is any one of Table 4, but preferably triacetin, diethylene glycol monoethyl ether or ethyl oleate.

In a particular embodiment, the invention relates to the method according to the invention, wherein the method comprises a further step of lyophilizing the suspension comprising the RPCs or the enriched RPC suspension to obtain a lyophilisate.

In an additional step, the suspension comprising the RPCs or the enriched RPC suspension may be lyophilized. It has been shown by the inventors that lyophilized RPCs can be reconstituted such that they efficiently dissociate after storage at 5° C., 25° C. or 40° C. for at least 4 weeks (Table 12). In addition, lyophilized RPCs remained stable for at least 4 weeks without significant formation of protein aggregates or degradation of the protein (Table 13). Thus, lyophilisates that have been obtained from suspensions comprising RPCs or from enriched RPC suspensions are suitable for storing therapeutic proteins for longer time periods without the need for an intact cooling chain.

The term “lyophilization” as used herein refers to a “freeze-dry” process comprising the conversion of water from a frozen state to a gaseous state without going through a liquid state. The freeze-dry process removes moisture from a water-containing material while the material remains frozen. The basic process of lyophilization comprises the following steps: freezing, primary drying (sublimation), and secondary drying (desorption). At first, a dissolved and/or suspended substance is frozen at a low temperature (for example, -60° C.). Slow freezing produces larger crystals which allow the sublimating material to escape. Some products form a glassy material and annealing may be required during the freezing process. Annealing, first lowering the temperature then raising the temperature and then lowering it again, locks the constituents in place and then allows the crystals to grow. Freezing can range from 1 hour to 24 hours, depending on the application. In step 2 (primary drying) the water or diluent is then extracted via vacuum, resulting in a porous, dry “cake”. Sublimation occurs under vacuum with the product temperature below its critical temperature. This is typically the longest process. At the end of the primary drying cycle, the product will have 3 to 5% moisture content. There is a final drying step (secondary drying) to remove residual unfrozen water molecules. This is done by heating the product. Secondary drying can result in moisture levels of about 0.5%. The term “lyophilisate” refers to the freeze-dried product of a lyophilisation step.

In a particular embodiment, the invention relates to the method according to the invention, wherein at least one cryoprotectant is added to the suspension comprising the RPCs or the enriched RPC suspension before the lyophilisation step.

To protect the proteins comprised in the RPCs from damage during the lyophilisation step, a cryoprotectant may be added to the suspension before the lyophilisation step.

The term “cryoprotectant” is herein used analogously with the term “lyoprotectant” to describe molecules that protect freeze-dried material. Known as lyoprotectants, these molecules are typically polyhydroxy compounds such as sugars (mono-, di-, and polysaccharides), polyalcohols, and their derivatives. Trehalose and sucrose are natural lyoprotectants. The term “lyoprotectant” as used herein, includes agents that provide stability to a biologically active compound during the drying process, e.g., by providing an amorphous glassy matrix and by binding with a protein through hydrogen bonding, replacing the water molecules that are removed during the drying process. This helps to maintain a protein’s conformation, minimize protein degradation during the drying cycle, and improve the long-term product stability. Nonlimiting examples of lyoprotectants include sugars, such as sucrose or trehalose; an amino acid, such as monosodium glutamate, non-crystalline glycine or histidine; a methylamine such, as betaine; a lyotropic salt, such as magnesium sulfate; a polyol, such as trihydric or higher sugar alcohols, e.g., glycerin, erythritol, glycerol, arabitol, xylitol, sorbitol, and mannitol; propylene glycol; polyethylene glycol; pluronics; and combinations thereof. The amount of lyoprotectant added to a formulation is generally an amount that does not lead to an unacceptable amount of degradation/aggregation of the protein when the protein formulation is dried. Thus, in a particular embodiment, the invention relates to the method according to the invention, wherein the at least one cryoprotectant is selected from a group consisting of: sugars, amino acids, methylamines, lyotropic salts, polyols, propylene glycol, polyethylene glycol and pluronics. In certain embodiments, the cryoprotectant is a sugar, in particular sucrose.

In a particular embodiment, the invention relates to the method according to the invention, wherein the protein concentration of the suspension comprising the RPCs or the enriched RPC suspension is adjusted to 10 to 100 mg/mL, in particular to 40 to 80 mg/mL, prior to the lyophilisation step.

The lyophilization step may be performed with any suspension. It has been demonstrated by the inventors that lyophilizing enriched RPC suspensions with a protein concentration of 60 mg/mL does not significantly reduce the stability and/or the dissociation efficiency of the RPCs. Thus, it is preferred that the lyophilization step is performed with a suspension comprising RPCs in which the protein concentration is adjusted to 10 - 100 mg/mL. More preferably, the protein concentration in the suspension comprising the RPCs is adjusted to 40 - 80 mg/mL. Most preferably, the protein concentration in the suspension comprising the RPCs is adjusted to 60 mg/mL.

In a particular embodiment, the invention relates to the method according to the invention, wherein the method comprises a further step of spray drying the suspension comprising the RPCs or the enriched RPC suspension to obtain a spray dried powder.

Besides lyophilization, the suspension comprising RPCs or the enriched RPC suspension may be spray dried to obtain a solid composition, in this case a spray dried powder.

The term “spray-drying”, as used herein, refers to a method of producing a dry powder comprising micron-sized particles from a solution or suspension by using a spray-dryer. Spray-drying is, in principle, a solvent extraction process. The constituents of the product to be obtained are dissolved/dispersed in a liquid and then fed, for example by using a peristaltic pump, to an atomiser of a spray-dryer. A suitable atomizer which can be used for atomization of the liquid, include nozzles or rotary discs. With nozzles, atomization occurs due to the action of the compressed gas, while in case of using rotary discs atomization occurs due to the rapid rotation of the disc. In both cases, atomization leads to disruption of the liquid into small droplets into the drying chamber, wherein the solvent is extracted from the aerosol droplets and is discharged out, for example through an exhaust tube to a solvent trap.

Drop sizes from 1 to 500 pm may be generated by spray-drying. As the solvent (water or organic solvent) dries, the nanoparticles-containing droplets dries into a micron-sized particle, forming powder-like particles.

A number of commercially available spray drying machines can be used to prepare the composition of the invention, for example, suitable machines are manufactured by Buchi and Niro. Examples of suitable spray-driers include lab scale spray-dryers from Buchi, such as the Mini Spray Dryer 290, or a MOBILE MINOR™, or a Pharma Spray Dryer PharmaSD® from Niro, or a 4M8-TriX from Procept NV.

In a typical spray drying machine the suspension to be dried is pumped from a stirred reservoir to an atomization chamber where it is sprayed from a nozzle as fine droplets into a stream of heated air, for example, inlet temperatures in the range of 50 to 250° C. (nitrogen can be used in place of air if there is a risk of undesirable oxidation of the product). The temperature of the heated air must be sufficient to evaporate the liquid and dry the microparticles to a free flowing powder but should not be so high as to degrade the product. The microparticles may be collected in a cyclone or a filter or a combination of cyclones and filters.

The suspension comprising RPCs or the enriched RPC suspension may be adjusted to a specific protein concentration before the spray-drying step. That is, in a particular embodiment, the invention relates to the method according to the invention, wherein the protein concentration of the suspension comprising the RPCs or the enriched RPC suspension is adjusted to 1 to 10 mg/mL, in particular to 1 to 5 mg/mL, prior to the spray drying step.

It has been shown by the inventors that the protein concentration in the suspension has an influence on the size of the spray dried particles. Particles with a smaller size may facilitate injectability of the spray-dried powder when resuspended in a liquid. Thus, in certain embodiments, it is preferred that the protein concentration of the suspension comprising the RPCs or the enriched RPC suspension is lower than 10 mg/mL, 9 mg/mL, 8 mg/mL, 7 mg/mL, 6 mg/mL, 5 mg/mL, 4 mg/mL, 3 mg/mL, 2 mg/mL or 1 mg/mL.

In a particular embodiment, the invention relates to the method according to the invention, wherein the liquid fraction of the suspension comprising the RPCs or the enriched RPC suspension is exchanged prior to the spray drying step.

That is, the liquid phase of the suspension comprising RPCs or the enriched RPC suspension may be exchanged before the spray-drying step. Exchange of the liquid phase of the suspension may be achieved, without limitation, by dialysis.

The term “dialysis” as used herein refers to the diffusion of dissolved solutes across a selectively permeable membrane against a concentration gradient in an effort to achieve equilibrium. While small solutes pass through the membrane larger solutes and particles, such as RPCs, are trapped on one side. By exchanging the dialysate buffer on the outside side of the membrane, smaller solutes can be continuously removed to purify the trapped larger molecules.

Several rounds of dialysis may be used for buffer exchange. In general, dialysis will be most effective when the buffer is replaced multiple times, for example 2, or 3 times, and then preferably left overnight at room temperature on a stir plate. A standard protocol for dialysis is 16 to 24 hours. Many factors affect the dialysis rate, including: diffusion coefficients, pH, temperature, time, concentration of species, sample volume, dialysate (buffer) volume, number of dialysate changes, membrane surface area, membrane thickness, molecular charges and dialysate agitation (stirring). Several types of membranes for dialysis are commercially available and are well known in the art. Illustrative non limiting examples are Polyvinylidene Difluoride (PVDF) membranes, cellulose ester (CE) membranes and regenerated cellulose (C) membranes.

In a particular embodiment, the invention relates to the method according to the invention, wherein exchanging the liquid fraction of the suspension comprising the RPCs or the enriched RPC suspension reduces the concentration of at least one buffering agent, complexing agent, stabilizer and/or solubilizer in the suspension.

In certain embodiments, it is desirable that the protein content in the spray dried powder is as high as possible. Thus, it may be desired to reduce the non-protein components in the suspension before the spray drying step.

It has been demonstrated by the inventors in Example 2.2 that a spray dried powder wherein the integrity of the protein is not significantly compromised may be obtained in the absence of a buffering agent. Thus, in a particular embodiment, the invention relates to the method according to the invention, wherein the suspension comprising the RPCs or the enriched RPC suspension is substantially free of buffering agent after exchanging the liquid fraction of the suspension. In certain embodiments, the RPCs may be dialysed against ultrapure water before the spray drying step.

It is to be understood that due to the dilution effect during dialysis the buffering agent can never be completely removed from the liquid fraction of the suspension. Thus, a suspension is said to be substantially free of buffering agent if the concentration of the buffering agent in the suspension before the spray-drying step is below 5 mM, below 4 mM, below 3 mM, below 2 mM, below 1 mM, below 0.5 mM, below 0.1 mM or below 0.01 mM.

In a particular embodiment, the invention relates to the method according to the invention, wherein exchanging the liquid fraction of the suspension comprising the RPCs or the enriched RPC suspension reduces the concentration the buffering agent in the suspension comprising the RPCs or the enriched RPC suspension by at least 10%, at least 20%, at least 30%, at least 40%, at least 50%, at least 60%, at least 70%, at least 80%, at least 90, at least 95% or at least 99%.

Further, it has been demonstrated in Example 2.2 that the protein content in the spray dried powder can be increased by reducing the concentration of the complexing agent, the surfactant and/or the stabilizer in the suspension before the spray drying step without compromising the stability of the protein.

That is, in certain embodiments, the invention relates to the method according to the invention, wherein the liquid fraction of the suspension comprising the RPCs or the enriched RPC suspension is exchanged before the spray-drying step to reduce the concentration of the complexing agent in the suspension comprising the RPCs or the enriched RPC suspension by at least 10%, at least 20%, at least 30%, at least 40%, at least 50%, at least 60%, at least 70%, at least 80%, at least 90, at least 95% or 100%.

In a preferred embodiment, the invention relates to the method according to the invention, wherein the liquid fraction of the suspension comprising the RPCs or the enriched RPC suspension is exchanged before the spray-drying step to reduce the concentration of the complexing agent in the suspension comprising the RPCs or the enriched RPC suspension by 20 - 50%, more preferably by 30 - 40%, most preferably by 33%.

In further embodiments, the invention relates to the method according to the invention, wherein the liquid fraction of the suspension comprising the RPCs or the enriched RPC suspension is exchanged before the spray-drying step to reduce the concentration of the stabilizer in the suspension comprising the RPCs or the enriched RPC suspension by at least 10%, at least 20%, at least 30%, at least 40%, at least 50%, at least 60%, at least 70%, at least 80%, at least 90%, at least 95% or 100%. In a preferred embodiment, the invention relates to the method according to the invention, wherein the liquid fraction of the suspension comprising the RPCs or the enriched RPC suspension is exchanged before the spray-drying step to reduce the concentration of the stabilizer in the suspension comprising the RPCs or the enriched RPC suspension by 30 - 70%, more preferably by 40 - 60%, most preferably by 50%.

In further embodiments, the invention relates to the method according to the invention, wherein the liquid fraction of the suspension comprising the RPCs or the enriched RPC suspension is exchanged before the spray-drying step to reduce the concentration of the solubilizer in the suspension comprising the RPCs or the enriched RPC suspension by at least 10%, at least 20%, at least 30%, at least 40%, at least 50%, at least 60%, at least 70%, at least 80%, at least 90%, at least 95% or 100%. In a preferred embodiment, the invention relates to the method according to the invention, wherein the liquid fraction of the suspension comprising the RPCs or the enriched RPC suspension is exchanged before the spray-drying step to reduce the concentration of the stabilizer in the suspension comprising the RPCs or the enriched RPC suspension by 30 - 70%, more preferably by 40 - 60%, most preferably by 50%.

Consequently, reducing the complexing agent in the suspension will result in a reduced mole-charge ratio between the protein and the complexing agent. Thus, in a particular embodiment, the invention relates to the method according to the invention, wherein the liquid fraction of the suspension is exchanged before the spray-drying step to obtain a mole-charge ratio between the protein and the complexing agent between 1:0.2 to 1:1, in particular between 1:0.4 to 1:0.8.

In a preferred embodiment, the invention relates to the method according to the invention, wherein the liquid fraction of the suspension is exchanged before the spray-drying step to obtain a mole-charge ratio between the protein and the complexing agent chondroitin sulfate between 1:0.2 to 1:1, more preferably between 1:0.2 to 1:0.6, even more preferably between 1:0.2 to 1:0.4, most preferably of about 1:0.2.

In another preferred embodiment, the invention relates to the method according to the invention, wherein the liquid fraction of the suspension is exchanged before the spray-drying step to obtain a mole-charge ratio between the protein and the complexing agent dextran sulfate between 1:0.2 to 1:1, more preferably between 1:0.4 to 1:0.8, even more preferably between 1:0.5 to 1:0.7, most preferably of about 1:0.6.

The spray dried powder may be obtained at any condition that does not result in aggregation or damaging of the proteins comprised in the RPCs. The skilled person is capable of optimizing spray drying conditions to prevent damaging and/or aggregation of the proteins by routine experimentation and is aware of methods to determine the stability of the proteins in the RPCs subsequent to the spray drying step (see Examples 1.2.8 and 2.2)

It has been demonstrated by the inventors in Example 2.2 that spray drying at an inlet temperature of 115° C., an outlet temperature of 48° C. and a flow rate of 17 mL/min results in the formation of a spray dried powder comprising RPCs, wherein the proteins comprised in the RPCs are stable.

Accordingly, spray drying may be performed at an inlet temperature ranging from 50 - 250° C., preferably 100 - 200° C., more preferably 100 - 150° C., even more preferably 100 - 130° C., most preferably 115° C.

Further, spray drying may be performed at an outlet temperature ranging from 40 - 150° C., preferably 40 - 100° C., more preferably 40 - 80° C., even more preferably 40 - 60° C., most preferably 48° C.

Further, spray drying may be performed at a flow rate ranging from 1 - 35 mL/min, preferably 5 - 30 mL/min, more preferably 10 - 25 mL/min, even more preferably 15 - 20 mL/min, most preferably 17 mL/min.

In a particular embodiment, the invention relates to the method according to the invention, wherein spray drying is performed at an inlet temperature ranging from 90 - 250° C., an outlet temperature ranging from 40 - 150° C. and/or a flow rate ranging from 1 - 35 mL/min.

In a preferred embodiment, the invention relates to the method according to the invention, wherein spray drying is performed at an inlet temperature ranging from 100 - 200° C., an outlet temperature ranging from 40 - 100° C. and/or a flow rate ranging from 5 - 30 mL/min.

In a more preferred embodiment, the invention relates to the method according to the invention, wherein spray drying is performed at an inlet temperature ranging from 100 - 150° C., an outlet temperature ranging from 40 - 80° C. and/or a flow rate ranging from 10 - 25 mL/min.

In an even more preferred embodiment, the invention relates to the method according to the invention, wherein spray drying is performed at an inlet temperature ranging from 100 - 130° C., an outlet temperature ranging from 40 - 60° C. and/or a flow rate ranging from 15 - 20 mL/min.

In an most preferred embodiment, the invention relates to the method according to the invention, wherein spray drying is performed at an inlet temperature of 115° C., an outlet temperature of 48° C. and/or a flow rate of 17 mL/min.

In a particular embodiment, the invention relates to the method according to the invention, wherein spray drying is performed at an inlet temperature 115° C. and/or an outlet temperature of 48° C.

In a particular embodiment, the invention relates to the method according to the invention, wherein spray drying is performed at a feed rate of 17 mL/min.

In a particular embodiment, the invention relates to the method according to the invention, wherein the method comprises a further step of re-suspending the spray dried powder in a non-aqueous solvent (NAS) to obtain an RPC-NAS suspension.

That is, the spray dried powder comprising the RPCs may be resuspended in a non-aqueous solvent (NAS) to obtain an RPC-NAS suspension. It has been demonstrated by the inventors in Example 2.3 that spry dried powders comprising RPCs can be resuspended in non-aqueous solvents to obtain a suspension with high protein concentration. In particular, it has been demonstrated that the viscosity of RPC-NAS suspensions is significantly lower compared to the suspensions that have been obtained by re-suspending spray-dried RPCs in aqueous solvents.

The term “RPC-NAS suspension” refers to a heterogenous mixture comprising solid particles, in this case RPCs, and a liquid phase. The liquid phase consists of or comprises at least one non-aqueous solvent.

In a particular embodiment, the invention relates to the method according to the invention, wherein the non-aqueous solvent is at least one selected from a group consisting of: diethylene glycol monoethyl ether, ethyl oleate, triacetin, isosorbide dimethyl ether and glycofurol.

The non-aqueous solvent in which the spray-dried RPCs are re-suspended may be any non-aqueous solvent known in the art that retains the protein in a stable and non-aggregated form and does not interfere with subsequent dissociation of the RPCs at physiological conditions. Further, it is preferred that the NAS is a NAS that is contained in the EU and/or US pharmacopeia.

Diethylene glycol monoethyl ether (CAS Number 111-90-0; C₂H₅OCH₂CH₂OCH₂CH₂OH), also known under the trade name Transcutol®, is a liquid which has a long history of use in cosmetic and over-the-counter topically applied products. Transcutol® has been applied in several commercial preparations and is used in many studies as the main ingredient of formulations.

Ethyl oleate (CAS Number 111-62-6, CH₃(CH₂)₇CH═CH(CH₂)₇COOC₂H₅) is a fatty acid ester formed by the condensation of oleic acid and ethanol. Ethyl oleate is used as a solvent for pharmaceutical drug preparations involving lipophilic substances such as steroids. It also finds use as a lubricant and a plasticizer.

The triglyceride 1,2,3-triacetoxypropane (CAS Number 102-76-1, C₉H₁₄O₆) is more generally known as triacetin, glycerin triacetate or 1,2,3-triacetylglycerol. It is the triester of glycerol and acetylating agents, such as acetic acid and acetic anhydride. It is an artificial chemical compound, commonly used as a food additive, for instance as a solvent in flavourings, and for its humectant function, with E number E1518 and Australian approval code A1518. It is used as an excipient in pharmaceutical products, where it is used as a humectant, a plasticizer, and as a solvent.

Isosorbide dimethyl ether (CAS Number 5306-85-4; C₈H₁₄O₄) is a sustainable solvent that is widely used in various cosmetic and pharmaceutical formulation.

Glycofurol (CAS Number: 31692-85-0), also known as tetraglycol or tetrahydrofurfuryl alcohol polyethyleneglycol ether, is a non-aqueous solvent that is frequently used as a solvent in parenteral products for intravenous or intramuscular injection.

It has been demonstrated by the inventors in Example 2.3 that transcutol, ethyl oleate and triacetin are particularly suitable for the production of RPC-NAS suspensions, as they result in high protein stability and only low levels of protein aggregation. Thus, in a preferred embodiment, the invention relates to the method according to the invention, wherein the non-aqueous solvent is at least one selected from a group consisting of: diethylene glycol monoethyl ether, ethyl oleate and triacetin.

In a particular embodiment, the invention relates to the method according to the invention, wherein the spray dried powder is resuspended to obtain an RPC-NAS suspension with a protein concentration ranging from 50 to 300 mg/mL, preferably 100 - 250 mg/mL.

The skilled person is aware of methods for producing RPC-NAS suspensions with specific protein concentrations. In particular, RPC-NAS suspensions with a specific protein concentration may be produced by re-suspending a defined amount of spray dried RPCs in a defined amount of a non-aqueous solvent.

In another aspect, the invention relates to a composition comprising reversible protein complexes (RPCs), wherein the composition is obtained by the method according to the invention.

That is, the invention relates to a composition comprising RPCs that has been obtained with any of the methods described above in any combination. In particular, the invention relates to a composition comprising RPCs, wherein the RPCs comprise the complexing agent dextran sulfate and/or chondroitin sulfate.

Thus, in another aspect, the invention relates to a composition comprising reversible protein complexes (RPCs), wherein the RPCs comprise a protein and a complexing agent, and wherein the complexing agent is dextran sulfate or chondroitin sulfate. In a preferred embodiment, the invention relates to the composition according to the invention, wherein the complexing agent is dextran sulfate, in particular dextran sulfate with 40 kDa molecular weight.

That is, the complexing agent may be any one of the complexing agents that have been disclosed above.

In a particular embodiment, the invention relates to the composition according to the invention, wherein the protein is an antibody, a growth factor, a hormone, a cytokine, an enzyme, or a fragment and/or fusion protein of any of the foregoing.

Further, the protein comprised in the RPCs may be any one of the proteins that have been disclosed above.

In a particular embodiment, the invention relates to the composition according to the invention, wherein the antibody is a monoclonal antibody, a polyclonal antibody, a chimeric antibody, a multispecific antibody, an antibody fusion protein, an antibody-drug-conjugate or an antibody fragment.

The protein which is comprised in the composition of the invention is preferably essentially pure and desirably essentially homogeneous (i.e. free from contaminating proteins). “Essentially pure” means that at least 70%, at least 75%, at least 80%, at least 85%, at least 90%, at least 95%, at least 96%, at least 97%, at least 98%, at least 99% of the proteins in the composition of the invention have an identical amino acid sequence. Correspondingly, it is preferred that at least 70%, at least 75%, at least 80%, at least 85%, at least 90%, at least 95%, at least 96%, at least 97%, at least 98%, at least 99% of the proteins comprised in the RPCs of the composition of the invention have an identical amino acid sequence. That is, the composition of the invention may comprise more than one type of protein.

The RPCs in the composition are stabilized mainly by electrostatic interactions. Thus, it is preferred that the RPCs are formed between a positively charged protein and one or more negatively charged complexing agents. Thus, in a particular embodiment, the invention relates to the composition according to the invention, wherein the protein has a positive net charge when comprised in the RPCs. In another embodiment, the invention relates to the composition according to the invention, wherein the complexing agent has a negative charge when comprised in the RPCs.

The composition of the invention may comprise one or more excipients, in particular a stabilizer and/or a surfactant. Thus, in a particular embodiment, the invention relates to the composition according to the invention, wherein the composition comprises at least one excipient. In a preferred embodiment, the invention relates to the composition according to the invention, wherein the at least one excipient is a stabilizer and/or a surfactant. Examples of excipients, stabilizers and solubilizers are given above.

It has been demonstrated by the inventors that the thermal stability of proteins is higher when the protein is comprised in an RPC. Thus, in a particular embodiment, the invention relates to the composition according to the invention, wherein the protein has a higher melting temperature when comprised in the RPC compared to the uncomplexed protein.

The melting temperature of a protein, also referred to as the denaturation midpoint, is defined as the temperature (Tm) at which both the folded and unfolded states are equally populated at equilibrium (assuming two-state protein folding). Tm may be determined using a thermal shift assay. Different thermal shift assays for determining the Tm of a protein, namely nano differential scanning fluorimetry (nanoDSF) and differential scanning calorimetry (DSC) are described in more detail in Example 1.9.

A protein is said to be in an uncomplexed state if it is not part of a complex with a complexing agent, in particular, if it is not part of a complex with the complexing agents chondroitin sulfate and/or dextran sulfate.

In a particular embodiment, the invention relates to the composition according to the invention, wherein the RPCs comprising the protein and the complexing agent dissociate at physiological pH and ionic strength.

It was surprisingly found by the inventors that RPCs comprising dextran sulfate and/or chondroitin sulfate as the complexing agents dissociate at physiological pH and ionic strength. Thus, liquid formulations comprising the RPCs of the invention may be administered to a subject such that they dissociate at the site of administration, consequently resulting in the release of the uncomplexed protein in said subject.

The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

The term “physiological ionic strength”, as used herein, refers to the normal pH in the cells of the tissues and organs of the mammalian body. For instance, the physiological ionic strength of a human is normally approximately 0.15 mol/L.

Accordingly, a protein complex is said to dissociate at physiological pH and ionic strength, if at least 85%, at least 90%, at least 91%, at least 92%, at least 93%, at least 94%, at least 95%, at least 96%, at least 97%, at least 98%, at least 99% of the complexes in a sample dissociate into soluble proteins at physiological pH and ionic strength.

Within the present invention, physiological conditions have been simulated by using phosphate buffered saline (PBS) with a pH of 7.4 and an ionic strength of 0.137 mol/L. It has been demonstrated by the inventors that RPCs comprising different proteins dissociate in PBS (pH 7.4, 137 mM NaCl) with a dissociation efficiency of at least 96% (see Example 3.1). Hence, it is plausible that the reversible protein complexes of the present invention dissociate at physiological conditions and thus may be directly applied to a subject in an appropriate formulation and via an appropriate route of administration, e.g., subcutaneous administration.

Thus, in a particular embodiment, the invention relates to the composition according to the invention, wherein the RPCs comprising the protein and the complexing agent dissociate in 10 mM to 100 mM PBS (pH 7.4, 137 mM NaCl).

It has been shown by the inventors that diluting a suspension comprising RPCs in 10 mM or 100 mM PBS (pH 7.4, 137 mM to 1370 mM NaCl) to a protein concentration of 1 mg/mL results in dissociation of the RPCs. Thus, in a preferred embodiment, the invention relates to the composition according to the invention, wherein the RPCs comprising the protein and the complexing agent dissociate in 10 mM PBS (pH 7.4, 137 mM NaCl) when diluted to a protein concentration ranging from 0.1 to 10 mg/mL, preferably ranging from 0.1 to 5 mg/mL, more preferably ranging from 0.1 to 2 mg/mL, most preferably 1 mg/mL.

In an alternative embodiment, the invention relates to the composition according to the invention, wherein the RPCs comprising the protein and the complexing agent dissociate in 100 mM PBS (pH 7.4, 137 mM NaCl) when diluted to a protein concentration ranging from 0.1 to 10 mg/mL, preferably ranging from 0.1 to 5 mg/mL, more preferably ranging from 0.1 to 2 mg/mL, most preferably 1 mg/mL.

In a particular embodiment, the invention relates to the composition according to the invention, wherein the composition is a suspension.

That is, in certain embodiments, the RPCs of the invention may be formulated as a suspension. In a particular embodiment, the invention relates to the composition according to the invention, wherein the suspension is obtained with the method according to the invention.

The suspension may have any protein concentration. However, it is preferred that the suspension of the invention is suitable for subcutaneous administration to a subject, which ideally requires protein a concentration in the suspension of at least 100 mg/mL. Thus, in a particular embodiment, the invention relates to the composition according to the invention, wherein the protein concentration in the suspension ranges from 50 to 250 mg/mL. In a preferred embodiment, the invention relates to the composition according to the invention, wherein the protein concentration in the suspension ranges from 100 to 200 mg/mL.

Methods for obtaining suspensions with the claimed protein concentrations and methods for determining the protein concentration in a suspension are disclosed herein. Methods for determining the protein concentration in a suspension are described herein. For example, the protein concentration in a suspension may be determined by preparing serial dilutions of the suspension in 10 mM PBS (pH 7.4, 137 mM NaCl) and by measuring the protein concentration in the dilutions by any methods known in the art, such as UV-Vis spectrophotometry (NanoDrop) or Bradford assay. Suspensions with the desired protein concentration may be obtained by enriching or diluting the suspensions as disclosed herein.

In a particular embodiment, the invention relates to the composition according to the invention, wherein the suspension comprises uncomplexed complexing agent.

That is, in certain embodiments, the suspension of the invention may comprise an excess of complexing agent, preferably wherein the uncomplexed complexing agent is dissolved in the liquid fraction of the suspension. In certain embodiments, the uncomplexed complexing agent is the same complexing agent that is comprised in the RPCs. In certain embodiments, the complexing agent is comprised in the liquid fraction of the suspension at a concentration ranging from 0.1 - 100 mM, preferably ranging from 0.1 - 10 mM, more preferably ranging from 0.1 - 1 mM, most preferably ranging from 0.2 - 0.6 mM.

Preferably, the complexing agent is dextran sulfate or chondroitin sulfate. That is, in certain embodiments, the liquid fraction of the suspension comprises dextran sulfate at a concentration ranging from 0.1 - 100 mM, preferably ranging from 0.1 - 10 mM, more preferably ranging from 0.1 - 1 mM, most preferably ranging from 0.2 - 0.6 mM. In other embodiments, the liquid fraction of the suspension comprises chondroitin sulfate at a concentration ranging from 0.1 -100 mM, preferably ranging from 0.1 - 10 mM, more preferably ranging from 0.1 - 1 mM, most preferably ranging from 0.2 - 0.6 mM.

The additional complexing agent may be added to the suspension after the formation of the RPCs by adding the complexing to the suspension or when exchanging the liquid fraction of the suspension.

The RPCs in the suspension of the invention may have any particle size. However, it is assumed that suspensions comprising smaller RPCs have a higher injectability compared to suspension comprising larger RPCs.

Thus, in a particular embodiment, the invention relates to the composition according to the invention, wherein the RPCs comprised in the suspension have a mean particle size ranging from 5 to 20 µm. In a preferred embodiment, the invention relates to the composition according to the invention, wherein the RPCs comprised in the suspension have a mean particle size ranging from 6 to 12 µm.

It has been surprisingly found by the inventors that dialysis of RPCs against ultrapure water results in particularly small RPCs (see e.g. FIGS. 20A-20B). That is, in a particular embodiment, the invention relates to the composition according to the invention, wherein the RPCs comprised in the suspension have a mean particle size ranging from 100 to 4000 nm, in particular wherein the RPCs comprised in the suspension have a mean particle size ranging from 150 to 2000 nm.

In certain embodiments, the invention relates to the composition according to the invention, wherein the RPCs comprised in the suspension have a mean particle size ranging from 0.1 to 20 µm, in particular wherein the RPCs comprised in the suspension have a mean particle size ranging from 0.1 to 12 µm.

The term “mean particle size”, as used herein, generally refers to the statistical mean particle size (diameter) of the particles in the composition. The diameter of an essentially spherical particle may be referred to as the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering, laser diffraction analysis (see Example 1.2.5) or scanning electron microscopy (SEM).

It is preferred that the suspension comprising the RPCs is directly applied to a subject by means of injection. That is, the suspension of the invention is preferably injectable through needles commonly used in the art. Thus, in a particular embodiment, the invention relates to the composition according to the invention, wherein the suspension is injectable through a 26G needle.

The term “26G needle” as used herein refers to a 26 gauge needle. The term “gauge”, as used herein, is meant to provide referrals to inner and outer diameters of the common numerical gauge system used for syringe needles. Advantageously, compositions of the present invention can be injected through small gauge needles having a gauge that is at least 26. A 26G needle has an outer diameter of 0.4636 mm.

In a particular embodiment, the invention relates to the composition according to the invention, wherein the suspension is stable for at least 4 weeks at 4° C. and/or 25° C.

Within the present invention, a suspension comprising RPCs is said to be stable, if the protein that is comprised in the RPCs, after dissociation of the RPCs, retains its original size, structure and/or function.

It has been demonstrated by the inventors that the suspension remains stable for at least 4 weeks at 4° C. and/or 25° C. (see Example 5.2). For example, only low levels of protein degradation and/or aggregation were observed after storage of the suspension for at least 4 weeks at 4° C. or 25° C. (Table 13).

In a preferred embodiment, a suspension is said to be stable if at least 70%, at least 75%, at least 80%, at least 85%, at least 90%, at least 91%, at least 92%, at least 93%, at least 94%, at least 95% of the proteins in the suspension are in monomeric form after dissociation of the RPCs in the suspension, preferably when measured by size exclusion chromatography (SEC).

Alternatively, a suspension is said to be stable, if the main peak percentage for the protein differs by not more than 1%, 2%, 3%, 4%, 5%,. 6%, 7%, 8%, 9%, 10%, 11%, 12%, 13%, 14%, 15%, 16%, 17%, 18%, 19%, 20% when analyzed by ion exchange chromatography (IEC) and compared before the formation of the RPC and after dissociation of the RPC.

In a particular embodiment, the invention relates to the composition according to the invention, wherein the suspension has a viscosity ranging from 2 to 20 centipoise (cP), in particular ranging from 3 to 15 cP, when measured at 20° C.

Within the present invention, it is preferred that the suspension has a low viscosity to facilitate injectability of the suspension. It has been shown by the inventors that suspensions with a protein concentration as high as 120 mg/mL have a viscosity of less than 20 cP and can thus be injected through a 26G needle.

A centipoise is one millipascal-second (mPa·s) in SI units (1 cP = 10⁻²P = 10⁻³ Pa·s). Centipoise is properly abbreviated cP. Water has a viscosity of 0.0089 poise at 25° C., or 1 centipoise at 20° C. The viscosity of a fluid may be measured with a rheometer, as described in Example 1.2.7.

To ensure the integrity of the RPCs in the suspension, it is preferred that the suspension has a pH that is lower than the isoelectric point of the protein and higher than the pKa of the complexing agent.

Thus, in a particular embodiment, the invention relates to the composition according to the invention, wherein the pH of the suspension is lower than the isoelectric point of the protein.

In a particular embodiment, the invention relates to the composition according to the invention, wherein the pH of the suspension is 2 to 5 pH units lower than the isoelectric point of the protein, in particular wherein the pH of the suspension is 3 pH units lower than the isoelectric point of the protein.

That is, in certain embodiments, the pH of the suspension is adjusted to be lower than the isoelectric point of the protein that is comprised in the RPCs. A pH below the isoelectric point of the protein results in positively charged proteins and thus prevents dissociation of the RPCs in the suspension.

In certain embodiments the protein is an antibody. Antibodies have an isoelectric point ranging from 6 to 9. To maintain the antibody in a positive charge, it is preferred that the suspension has a pH ranging from 1 to 6, preferably from 4.5 to 5.5. Thus, in a particular embodiment, the invention relates to the composition according to the invention, wherein the pH of the suspension ranges from 1 to 6, in particular wherein the pH of the suspension ranges from 4.5 to 5.5.

In certain embodiments, the suspension comprises a buffering agent. The suspension may comprise any buffering agent that allows to maintain the RPCs in suspension without compromising the stability of the RPCs or the proteins comprised therein. Buffering agents that may be used in the composition of the present invention include, without limitation, formate, citrate, succinate, acetate, propionate, malate, pyridine, piperazine, cacodylate, succinate, MES, maleate, histidine, bis-tris, phosphate, ethanolamine, ADA and carbonate.

It has been demonstrated that suspensions comprising the buffering agent histidine remain stable for long periods and have a low viscosity, which allows injection of the suspension through a 26G needle. Thus, in a particular embodiment, the invention relates to the composition according to the invention, wherein the suspension comprises a buffering agent. In a preferred embodiment, the invention relates to the composition according to the invention, wherein the buffering agent is histidine or citrate.

In a particular embodiment, the invention relates to the composition according to the invention, wherein the suspension has an ionic strength ranging from 20 to 50 mM, in particular wherein the suspension has an ionic strength ranging from 20 to 30 mM.

In certain embodiments, the suspension comprises 20 to 50 mM histidine or citrate as buffering agent. Preferably, the suspension comprises 20 to 30 mM histidine or citrate as buffering agent.

In other embodiments, the suspension is free of a buffering agent. It has been demonstrated by the inventors that RPCs suspensions comprising only RPCs, a complexing agent and ultrapure (MilliQ) water can be prepared. Thus, in a particular embodiment, the invention relates to the composition according to the invention, wherein the suspension is substantially free of buffering agents.

A suspension is said to be substantially free of buffering agent if the concentration of the buffering agent in the suspension before the spray-drying step is below 5 mM, below 4 mM, below 3 mM, below 2 mM, below 1 mM, below 0.5 mM, below 0.1 mM or below 0.01 mM.

In a particular embodiment, the invention relates to the composition according to the invention, wherein the liquid fraction of the suspension consists of ultrapure (MilliQ) water. Ultrapure (MilliQ) water, when exposed to air, has a pH of approximately 5.5. Thus, suspensions, wherein the liquid fraction consists of ultrapure (MilliQ) water, preferably comprise RPCs comprising proteins with an isoelectric point of 6 or higher.

The skilled person is aware of methods to remove a buffering agent from a suspension comprising RPCs. For example, a suspension comprising RPCs may be dialyzed against a liquid that is free of buffering agents. The dialysis step may be repeated until the suspension is substantially free of buffering agents. A suspension is said to be substantially free of buffering agents, if the concentration of buffering agents in the suspension is below 1 mM, 0.5 mM, 0.2 mM, 0.1 mM, 0.01 mM.

In a particular embodiment, the invention relates to the composition according to the invention, wherein the suspension further comprises a non-aqueous solvent.

In certain embodiments, the liquid fraction of the suspension may be a mixture of aqueous solvents and non-aqueous solvents. That is, the ratio between aqueous solvents and non-aqueous solvent may be 90:10, 80:20, 70:30, 60:40, 50:50, 40:60, 30:70, 20:80, 10:90.

The suspension may comprise any non-aqueous solvent that allows to maintain the RPCs in the suspension without comprising the stability of the RPCs or the proteins comprised therein. In a particular embodiment, the invention relates to the composition according to the invention, wherein the non-aqueous solvent is any one of Table 4, but preferably triacetin, diethylene glycol monoethyl ether or ethyl oleate.

In a particular embodiment, the invention relates to the composition according to the invention, wherein the composition is a lyophilisate.

That is, in certain embodiments, the invention relates to a lyophilisate comprising the RPCs of the invention. In a particular embodiment, the invention relates to the composition according to the invention, wherein the lyophilisate is obtained with the method according to the invention.

The term “lyophilisate” as used herein in connection with the composition according to the invention denotes a formulation which is manufactured by freeze-drying methods known in the art per se. The solvent (e.g., water) is removed by freezing following sublimation under vacuum and desorption of residual water at elevated temperature. In the pharmaceutical field, the lyophilisate has usually residual moisture of about 0.1 to 5% (w/w) and is present as a powder or a physical stable cake. The lyophilisate is characterized by a fast dissolution after addition of a reconstitution medium.

In a particular embodiment, the invention relates to the composition according to the invention, wherein the lyophilisate comprises a buffering agent.

That is, the lyophilisate may comprise a buffering agent. Preferably, the lyophilisate is obtained by freeze-drying the suspension of the invention. Thus, in a preferred embodiment, the lyophilisate comprises the same buffering agent as the suspension of the invention. More preferably, the buffering agent is histidine or citrate. Thus, in a preferred embodiment, the invention relates to the composition according to the invention, wherein the buffering agent is histidine or citrate.

In certain embodiments, the lyophilisate may be free or substantially free of buffering agents. In particular, the suspension comprising the RPCs may be dialysed against water before the lyophilization step.

Freeze-drying of a suspension may confer damage to the protein comprised in the RPCs. To prevent damage to the proteins during lyophilization, a cryoprotectant may be added to the suspension before the freeze-drying step. Thus, in a particular embodiment, the invention relates to the composition according to the invention, wherein the lyophilisate comprises at least one cryoprotectant. In a preferred embodiment, the invention relates to the composition according to the invention, wherein the at least one cryoprotectant is selected from a group consisting of: sugars, amino acids, methylamines, lyotropic salts, polyols, propylene glycol, polyethylene glycol and pluronics.

It has been shown by the inventors that the lyophilisate of the invention is stable for a prolonged time at elevated temperatures. For example, the lyophilisate of the invention has been stored for 4 weeks at 40° C. To the surprise of the inventors, it was shown that the RPCs comprised in the lyophilisate of the invention can be efficiently dissolved after 4 weeks at 40° C. and that the proteins comprised in the lyophilisate can be completely recovered (Table 12). Further, it was shown by the inventors that the protein comprised in the lyophilisate remains stable after storing the lyophilisate for 4 weeks at 40° C. Thus, in a particular embodiment, the invention relates to the composition according to the invention, wherein the lyophilisate is stable for at least 4 weeks at 40° C. In an alternative embodiment, the invention relates to the composition according to the invention, wherein the lyophilisate is stable for at least 4 weeks at 5° C. and/or 25° C.

Within the present invention, a lyophilisate comprising RPCs is said to be stable, if the protein that is comprised in the RPCs, after dissociation of the RPCs, retains its original size, structure and/or function.

Thus, in a preferred embodiment, a lyophilisate is said to be stable if at least 70%, at least 75%, at least 80%, at least 85%, at least 90%, at least 91%, at least 92%, at least 93%, at least 94%, at least 95% of the proteins in the lyophilisate are in monomeric form after reconstitution of the lyophilisate and dissociation of the RPCs in the reconstituted lyophilisate, preferably when measured by size exclusion chromatography (SEC). When analyzing if a protein is in monomeric form, the RPCs are preferably reconstituted and dissociated in PBS (pH 7.4, 137 mM NaCl).

Alternatively, a lyophilisate is said to be stable, if the main peak percentage for the protein differs by not more than 1%, 2%, 3%, 4%, 5%,. 6%, 7%, 8%, 9%, 10%, 11%, 12%, 13%, 14%, 15%, 16%, 17%, 18%, 19%, 20% when analyzed by ion exchange chromatography (IEC) and compared before the formation of the RPCs and after dissociation of the RPCs, preferably when the lyophilisate is reconstituted and dissociated in PBS (pH 7.4, 137 mM NaCl).

In a particular embodiment, the invention relates to the composition according to the invention, wherein the lyophilisate is reconstituted in a liquid to a protein concentration ranging from 50 to 250 mg/mL, in particular wherein the lyophilisate is reconstituted in a liquid to a protein concentration ranging from 100 to 200 mg/mL.

The lyophilisate of the invention may be reconstituted in a liquid before administration to a subject. Thus, in certain embodiments, the composition of the invention may be a reconstituted lyophilisate comprising the RPCs of the invention.

The term “reconstitute” as used herein, unless otherwise specified, refers to a process by which the lyophilisate is mixed with a liquid to form a liquid product. Once reconstituted in the liquid, the ingredients of the lyophilisate may be any combination of one or more of dissolved, dispersed, suspended, colloidally suspended, emulsified, or otherwise blended within the matrix of the liquid product. Therefore, the resulting reconstituted liquid product, may be characterized as any combination of a solution, a dispersion, a suspension, a colloidal suspension, an emulsion, or a homogeneous blend. A reconstituted composition may be said to be “reconstituted” even if a nominal portion (e.g., less than 10%) of the polyophilisate remains un-reconstituted in the resulting liquid product.

The lyophilisate of the invention may be reconstituted in any volume. Preferably, the lyophilisate of the invention may be reconstituted in a liquid such that a protein concentration ranging from 50 to 250 mg/mL is obtained in the reconstituted lyophilisate. Even more preferably, the lyophilisate of the invention is reconstituted in a liquid such that a protein concentration ranging from 100 to 200 mg/mL is obtained in the reconstituted lyophilisate.

Methods for obtaining reconstituted lyophilisates with the claimed protein concentrations and methods for determining the protein concentration in a reconstituted lyophilisate are disclosed herein. Methods for determining the protein concentration in a reconstituted lyophilisate are described herein. For example, the protein concentration in a reconstituted lyophilisate may be determined by preparing serial dilutions of the resuspended lyophilisate in 10 mM PBS (pH 7.4, 137 mM NaCl) and by measuring the protein concentration in the dilutions by any method known in the art, such as UV-Vis spectrophotometry (NanoDrop) or Bradford assay. Reconstituted lyophilisates with the desired protein concentration may be obtained by reconstituting the lyophilisate in a specific volume of a liquid.

Preferably, the lyophilisate of the invention is reconstituted in PBS, more preferably PBS (pH 7.4, 137 mM NaCl). Thus, in a particular embodiment, the invention relates to the composition according to the invention, wherein the liquid is PBS.

It has been shown by the inventors that the lyophilisate of the invention can be reconstituted in PBS to obtain a reconstituted lyophilisate with a protein concentration of at least 130 mg/mL (Table 12). Further, reconstituting the lyophilisate in PBS (pH 7.4, 137 mM NaCl) may result in the dissociation of the RPCs comprised in the lyophilisate. Thus, in certain embodiments, the reconstituted lyophilisate is a solution, preferably a solution comprising PBS, more preferably a solution comprising PBS and a protein concentration ranging from 50 to 250 mg/mL, most preferably a solution comprising PBS and a protein concentration ranging from 100 to 200 mg/mL.

The reconstituted lyophilisate may have a viscosity ranging from 2 to 20 cP, 3 to 20 cP, 4 to 20 cP, 5 to 20 cP, 6 to 20 cP, 7 to 20 cP, 8 to 20 cP, 9 to 20 cP, or 10 to 20 cP. Thus, in a particular embodiment, the invention relates to the composition according to the invention, wherein the resuspended lyophilisate has a viscosity ranging from 2 to 20 cP, in particular ranging from 10 to 20 cP.

In a particular embodiment, the invention relates to the composition according to the invention, wherein the composition is a spray dried powder.

That is, in certain embodiments, the RPCs of the invention may be formulated as a spray dried powder. In a particular embodiment, the invention relates to the composition according to the invention, wherein the spray dried powder is obtained with the method according to the invention.

It has been surprisingly shown by the inventors that spray drying of the suspension of the invention may result in a spray dried powder with a protein content of more than 40% (see Table 3). Thus, in a particular embodiment, the invention relates to the composition according to the invention, wherein the protein content of the spray dried powder is at least 40% by weight (w/w), at least 50% by weight (w/w), at least 60% by weight (w/w) .

The skilled person is aware of methods to determine the protein content of a spray dried powder. For example, the spray dried powder may be reconstituted in PBS to dissolve the RPCs comprised in the spray dried powder and the concentration of the re-solubilized protein may be determined by any method known in the art, such as Nanodrop, Bradford assay and so forth. The skilled person is able to calculate the protein content of the spray dried powder based on the protein concentration in the reconstituted spray dried powder.

In a particular embodiment, the invention relates to the composition according to the invention, wherein the spray dried powder comprises a buffering agent.

That is, the spray dried powder may comprise a buffering agent. Preferably, the spray dried powder is obtained by spray-drying the suspension of the invention. Thus, in a preferred embodiment, the spray dried powder comprises the same buffering agent as the suspension of the invention. More preferably, the buffering agent is histidine or citrate. Thus, in a particular embodiment, the invention relates to the composition according to the invention, wherein the buffering agent is histidine or citrate. The concentration of the buffering agent in the suspension before the spray drying step was shown to have an influence on the protein content in the spray dried powder (Table 3). That is, the higher the concentration of the buffering agent in the suspension before the spray drying step, the lower the protein content in the resulting spray dried powder. To increase the protein content in the spray dried powder, the concentration of the buffering agent in the suspension may be reduced before the spray drying step. For example, the concentration of the buffering agent may be reduced by at least 10%, at least 20%, at least 30%, at least 40%, at least 50%, at least 60%, at least 70%, at least 80%, at least 90% or 100% before the spray drying step. That is, in a particular embodiment, the invention relates to the composition according to the invention, wherein the spray dried powder is substantially free of buffering agents.

It has been demonstrated by the inventors that spray drying of a suspension that is free of the buffering agent histidine results in spray dried powders with a protein content of at least 60% (see Table 3). Thus, in a preferred embodiment, the invention relates to the composition according to the invention, wherein the spray dried powder has been obtained from a suspension comprising less than 20 mM, less than 15 mM, less than 10 mM, less than 5 mM, less than 1 mM or 0 mM of a buffering agent, in particular wherein the buffering agent is histidine or citrate.

Similarly, it has been shown by the inventors that reducing the concentration of the complexing agent and/or the excipients in the suspension before the spray drying step increases the protein content in the spray dried powder, without compromising the stability of the protein in the spray dried powder.

That is, in certain embodiments, the invention relates to the composition according to the invention, wherein the spray dried powder has been obtained from a suspension comprising less than 100 mg/mL, less than 90 mg/mL, less than 80 mg/mL, less than 70 mg/mL, or less than 60 mg/mL of a complexing agent, in particular wherein the complexing agent is dextran sulfate.

In other embodiments, the invention relates to the composition according to the invention, wherein the spray dried powder has been obtained from a suspension comprising less than 2.5 mg/mL, less than 2 mg/mL, less than 1.75 mg/mL, less than 1.5 mg/mL, or less than 1 mg/mL of a stabilizer, in particular wherein the stabilizer is sucrose.

In other embodiments, the invention relates to the composition according to the invention, wherein the spray dried powder has been obtained from a suspension comprising less than 0.5 mg/mL, less than 0.4 mg/mL, less than 0.3 mg/mL or less than 0.25 mg/mL of a solubilizer, in particular wherein the solubilizer is polysorbate 20.

In a more preferred embodiment, the invention relates to the composition according to the invention, wherein the spray dried powder has been obtained from a suspension comprising between 0 and 20 mM histidine, between 0.5 and 1 mg/mL dextrane sulphate, between 0.5 and 2.5 mg/mL sucrose, between 0.1 and 0.5 mg/mL polysorbate 20 and between 1 and 10 mg/mL RPCs.

In an even more preferred embodiment, the invention relates to the composition according to the invention, wherein the spray dried powder has been obtained from a suspension comprising between 0 and 10 mM histidine, between 0.5 and 0.7 mg/mL dextrane sulphate, between 0.5 and1.5 mg/mL sucrose, between 0.1 and 0.3 mg/mL polysorbate 20 and between 1 and 5 mg/mL RPCs.

The particles in the spray dried powder may have any size. However, it is preferred that the particles in the spray dried powder have a small particle size to facilitate injection of these particles. Thus, in a particular embodiment, the invention relates to the composition according to the invention, wherein the RPCs comprised in the spray dried powder have a mean particle size ranging from 5 to 50 µm. In a preferred embodiment, the invention relates to the composition according to the invention, wherein the RPCs comprised in the spray dried powder have a mean particle size ranging from 10 to 40 µm. In more preferred embodiment, the invention relates to the composition according to the invention, wherein the RPCs comprised in the spray dried powder have a mean particle size ranging from 20 to 35 µm.

In an alternative embodiment, the invention relates to the composition according to the invention, wherein the RPCs comprised in the spray dried powder have a mean particle size ranging from 10 to 25 µm.

Within the present invention, a spray dried powder comprising RPCs is said to be stable, if the proteins comprised in the RPCs, after re-suspension and/or dissociation of the RPCs, retain their original size, structure and/or function.

Thus, in a preferred embodiment, a spray dried powder is said to be stable if at least 70%, at least 75%, at least 80%, at least 85%, at least 90%, at least 91%, at least 92%, at least 93%, at least 94%, at least 95% of the proteins in the spray dried powder are in monomeric form after re-suspension and/or dissociation of the RPCs, preferably when measured by size exclusion chromatography (SEC). For analyzing if a protein is in monomeric state, the RPCs are preferably dissolved in PBS (pH 7.4, 137 mM NaCl).

Alternatively, a spray dried powder is said to be stable, if the main peak percentage for the protein differs by not more than 1%, 2%, 3%, 4%, 5%,. 6%, 7%, 8%, 9%, 10%, 11%, 12%, 13%, 14%, 15%, 16%, 17%, 18%, 19%, 20% when analyzed by ion exchange chromatography (IEC) and compared before the formation of the RPCs and after re-suspension and/or dissociation of the RPCs, preferably when the RPCs are dissolved in PBS (pH 7.4, 137 mM NaCl).

In a particular embodiment, the invention relates to the composition according to the invention, wherein the spray dried powder is reconstituted in a liquid.

The spray dried powder of the invention may be re-suspended in a liquid before administration to the subject. Thus, in certain embodiments, the composition of the invention may be a re-suspended spray dried powder comprising RPCs.

In a particular embodiment, the invention relates to the composition according to the invention, wherein the spray dried powder is re-suspended in a liquid to a protein concentration ranging from 50 to 300 mg/mL, in particular wherein the spray dried powder is reconstituted in a liquid to a protein concentration ranging from 100 to 250 mg/mL.

The spray dried powder of the invention may be re-suspended in any volume. Preferably, the spray dried powder of the invention is re-suspended in a liquid such that a protein concentration ranging from 50 to 300 mg/mL is obtained in the re-suspended spray dried powder. Even more preferably, the spray dried powder of the invention is re-suspended in a liquid such that a protein concentration ranging from 100 to 250 mg/mL is obtained in the re-suspended spray dried powder.

The spray dried powder may be resuspended in any liquid. However, it is preferred that the spray dried powder is re-suspended in a non-aqueous solvent such that an RPC-NAS suspension is obtained. Thus, in a preferred embodiment, the invention relates to the composition according to the invention, wherein the liquid is a non-aqueous solvent.

In a particular embodiment, the invention relates to the composition according to the invention, wherein the non-aqueous solvent is at least one selected from the solvents of Table 4, a group consisting of: diethylene glycol monoethyl ether, ethyl oleate, triacetin, isosorbide dimethyl ester and glycofurol, preferably triacetin, diethylene glycol monoethyl ether or ethyl oleate.

It has been shown by the inventors that re-suspending the RPCs in the non-aqueous solvents transcutol, ethyl oleate or triacetin results does not significantly compromise the stability of the protein comprised in the RPCs. Thus, in a preferred embodiment, the invention relates to the composition according to the invention, wherein the non-aqueous solvent is at least one selected from the solvents of Table 4, or a group consisting of: diethylene glycol monoethyl ether, ethyl oleate, triacetin, isosorbide dimethyl ester and glycofurol, preferably triacetin, diethylene glycol monoethyl ether or ethyl oleate.

Within the present invention, a liquid is suitable for re-suspending the spray dried powders of the invention, if the proteins comprised in the spray dried powder, after re-suspension of the RPCs, retain their original size, structure and/or function.

Thus, in a preferred embodiment, a liquid is said to be suitable for the re-suspension of the spray dried powder of the invention, if at least 70%, at least 75%, at least 80%, at least 85%, at least 90%, at least 91%, at least 92%, at least 93%, at least 94%, at least 95% of the proteins in the resuspended spray dried powder are in monomeric form after dissociation of the RPCs, preferably when measured by size exclusion chromatography (SEC). For analyzing if a protein is in monomeric state, the RPCs are preferably dissolved in PBS (pH 7.4, 137 mM NaCl).

Alternatively, a liquid is said to be suitable for the re-suspension of the spray dried powder of the invention, if the main peak percentage for the protein differs by not more than 1%, 2,%, 3%, 4%, 5%,. 6%, 7%, 8%, 9%, 10%, 11%, 12%, 13%, 14%, 15%, 16%, 17%, 18%, 19%, 20% when analyzed by ion exchange chromatography (IEC) and compared before the formation of the RPCs and after re-suspension and dissociation of the RPCs in said liquid, preferably when the RPCs are dissolved in PBS (pH 7.4, 137 mM NaCl).

Further, it has been shown that re-suspending the RPCs in a non-aqueous solvent results in lower viscosity when compared to a suspension comprising the same concentration of RPCs in an aqueous solvent (see Table 4). Thus, in a particular embodiment, the invention relates to the composition according to the invention, wherein the reconstituted spray dried powder has a viscosity ranging from 10 to 300 cP, preferably 20 to 80 cP, preferably 20 to 50 cP. In another embodiment, the invention relates to the composition according to the invention, wherein the reconstituted spray dried powder has a viscosity ranging from 50 to 300 cP. In another embodiment, the invention relates to the composition according to the invention, wherein the reconstituted spray dried powder has a viscosity ranging from 80 to 280 cP. In another embodiment, the invention relates to the composition according to the invention, wherein the reconstituted spray dried powder has a viscosity ranging from 100 to 250 cP.

In another aspect, the invention relates to a pharmaceutical formulation comprising the composition according to the invention.

In certain embodiments, the composition as such is a pharmaceutical formulation. In particular, the suspension comprising RPCs, the enriched RPC suspension, the reconstituted lyophilisate, or the RPC-NAS suspension may be used as pharmaceutical formulations.

In other embodiments, a pharmaceutical formulation may be obtained by bringing a solid composition into liquid form. That is, in certain embodiments, a pharmaceutical formulation may be obtained by reconstituting the lyophilisate of the invention in a suitable liquid, preferably in PBS (pH 7.4, 137 mM NaCl). In other embodiments, a pharmaceutical formulation may be obtained by re-suspending the spray dried powder of the invention in a suitable liquid, preferably a non-aqueous solvent.

The term “pharmaceutical formulation”, as used herein, refers to a dosage form comprising the reversible protein complex of the invention and at least one pharmaceutically accepted excipient. The term “pharmaceutically acceptable”, as used herein, means suited for normal pharmaceutical applications, i.e. giving rise to no adverse events in patients. The term “excipient” or “pharmaceutically acceptable excipient” as used herein means a nontoxic material that is compatible with the physical and chemical characteristics of the active pharmaceutical ingredient and does not interfere with the effectiveness of the biological activity of the active pharmaceutical ingredient, which is generally safe, non-toxic and neither biologically nor otherwise undesirable, and acceptable for veterinary use as well as human pharmaceutical use. An “excipient” or “pharmaceutically acceptable excipient” as used in the specification includes both one and more than one such excipient.

In certain embodiments, the pharmaceutical formulation is a liquid that may be administered to a subject via injection. Preferably, the pharmaceutical formulation may be administered subcutaneously, intramuscularly or transdermally. In certain embodiments, the pharmaceutical formulation may be administered ocularly.

In another aspect, the invention relates to the pharmaceutical formulation according to the invention for use as a medicament.

That is, the pharmaceutical formulation of the invention, in particular the suspension, the reconstituted lyophilisate or the re-suspended spray dried powder, may be used as a medicament. Thus, in a preferred embodiment, the pharmaceutical formulation of the invention is the suspension comprising RPCs of the invention, the enriched RPC suspension of the invention, the reconstituted lyophilisate of the invention or the RPC-NAS suspension of the invention.

The term “medicament” as used herein means a pharmaceutical formulation suitable for administration of a pharmaceutically active compounds, i.e., a therapeutic protein to a patient in need thereof.

In a preferred embodiment, the pharmaceutical formulation according to the invention is used as a medicament for the treatment of a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis. Thus, in a particular embodiment, the invention relates to the pharmaceutical composition according to the invention for use in the treatment of an autoimmune disease, an immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, or atherosclerosis

In certain embodiments, the pharmaceutical formulation according to the invention is used for the treatment of cancer. The term “cancer”, as used herein, refers to diseases in which abnormal cells divide without control and can invade nearby tissues, and includes, but is not restricted to, acute lymphoblastic leukemia, acute myelogenous leukemia, bladder cancer, bone sarcoma, breast cancer, cervical cancer, chorioadenoma destruens, choriocarcinoma, gastric cancer, Hodgkin lymphoma, hydatidiform mole, lung cancer, malignant mesothelioma, mycosis fungoides (a type of cutaneous T-cell lymphoma), neuroblastoma, non-Hodgkin lymphoma, non-small cell lung cancer, osteosarcoma, ovarian cancer, small cell lung cancer, soft tissue sarcoma, squamous cell carcinoma of the head and neck, testicular cancer, thyroid cancer, transitional cell bladder cancer, Wilms tumor and the like.

The pharmaceutically active ingredient in the pharmaceutical formulation of the invention is a protein. Preferably, the pharmaceutical formulation for use according to the invention comprises an antibody. That is, the RPCs comprised in the pharmaceutical formulation for use according to the invention may comprise an antibody and a complexing agent, in particular the complexing agent dextran sulfate or chondroitin sulfate. The skilled person is able to identify antibodies known in the art that can be used in the treatment of a specific disease.

However, in certain embodiments, the antibody may bind to any of the herein disclosed target molecules, for example CD proteins such as CD3, CD4, CD8, CD19, CD20 and CD34; members of the HER receptor family such as the EGF receptor, HER2, HER3 or HER4 receptor; cell adhesion molecules such as LFA-1, Mol, p150,95, VLA-4, ICAM-1, VCAM and αv/β3 integrin including either α or β subunits thereof (e.g. anti-CD11a, anti-CD18 or anti-CD11b antibodies); growth factors such as VEGF; IgE; blood group antigens; flk2/flt3 receptor; obesity (OB) receptor; protein C etc.

Exemplary antibodies that may be comprised in the pharmaceutical formulation of the invention include, but are not limited to, hRl (anti- IGF-1R, U.S. Pat. Application Serial No. 12/722,645, filed 3/12/10), hPAM4 (anti-mucin, U.S. Pat. No. 7,282,567), hA20 (anti-CD20, U.S. Pat. No. 7,251 ,164), hA19 (anti-CD19, U.S. Pat. No. 7,109,304), WMMU31 (anti-AFP, U.S. Pat. No. 7,300,655), hLLl (anti- CD74, U.S. Pat. No. 7,312,318), hLL2 (anti-CD22, U.S. Pat. No. 7,074,403), hMu-9 (anti-CSAp, U.S. Pat. No. 7,387,773), hL243 (anti-HLA-DR, U.S. Pat. No. 7,612,180), hMN-14 (anti-CEACAM5, U.S. Patent No. 6,676,924), hMN-15 (anti-CEACAM6, U.S. Pat. No. 7,541 ,440), hRS7 (anti-EGP-1 , U.S. Pat. No. 7,238,785), hMN-3 (anti- CEACAM6, U.S. Pat. No. 7,541 ,440), Abl24 and Abl 25 (anti-CXCR4, U.S. Pat. No. 7, 138,496).

Alternative antibodies that may be comprised in the pharmaceutical formulation of the invention include, but are not limited to, abciximab (anti- glycoprotein Ilb/IIIa), alemtuzumab (anti-CD52), bevacizumab (anti-VEGF), cetuximab (anti-EGFR), gemtuzumab (anti-CD33), ibritumomab tiuxetan (anti-CD20), panitumumab (anti-EGFR), rituximab (anti-CD20), tositumomab (anti-CD20), trastuzumab (anti-ErbB2), abagovomab (anti-CA-125), adecatumumab (anti-EpCAM), atlizumab (anti-IL-6 receptor), benralizumab (anti-CD125), CC49 (anti-TAG-72), AB-PG1 -XG1-026 (anti-PSMA, U.S. Pat. Application 11/983,372, deposited as ATCC PTA-4405 and PTA-4406), D2/B (anti- PSMA, WO 2009/130575), tocilizumab (anti-IL-6 receptor), basiliximab (anti-CD25), daclizumab (anti-CD25), efalizumab (anti-CDl 1 a), GA101 (anti-CD20; Glycart Roche), muromonab-CD3 (anti-CD3 receptor), natalizumab (anti-a4 integrin), omalizumab (anti- IgE); anti-TNF- a antibodies such as CDP571 (Ofei et al., 2011 , Diabetes 45:881 -85), MTNFAI, M2TNFAI, M3TNFAI, M3TNFABI, M302B, M303 (Thermo Scientific, Rockford, IL), infliximab (Centocor, Malvern, PA), certolizumab pegol (UCB, Brussels, Belgium), anti-CD40L (UCB, Brussels, Belgium), adalimumab (Abbott, Abbott Park, IL), Benlysta (Human Genome Sciences); antibodies for therapy of Alzheimer’s disease such as Alz 50 (Ksiezak-Reding et al., 1987, J Biol Chem 263:7943-47), gantenerumab, solanezumab and infliximab; anti-fibrin antibodies like 59D8, T2Gl s, MH1 ; anti-HIV antibodies such as P4/D10 (U.S. Pat. Application Serial No. 11/745,692), Ab 75, Ab 76, Ab 77 (Paulik et al., 1999, Biochem Pharmacol 58: 1781-90); and antibodies against pathogens such as CR6261 (anti-influenza), exbivirumab (anti-hepatitis B), felvizumab (anti-respiratory syncytial virus), foravirumab (anti-rabies virus), motavizumab (anti-respiratory syncytial virus), palivizumab (anti-respiratory syncytial virus), panobacumab (anti-Pseudomonas), rafivirumab (anti-rabies virus), regavirumab (anti-cytomegalovirus), sevirumab (anti-cytomegalovirus), tivirumab (anti-hepatitis B), and urtoxazumab (anti-E. coli).

In certain embodiments, the antibody may be a bispecific anti-VEGF/anti-angiopoietin-2 (Ang-2) antibody, an anti-alpha synuclein (aSyn) antibody, a bispecific anti-FAP/anti-OX40 antibody, a bispecific anti-VEGF/anti-PDGF antibody (dutafab), Bevacizumab, Pertuzumab or Gantenerumab.

Preferably, the pharmaceutical formulation of the invention may be used for subcutaneous, intramuscular or transdermal application to a subject. Thus, in a particular embodiment, the invention relates to the pharmaceutical formulation for use according to the invention, wherein the pharmaceutical formulation is administered subcutaneously, intramuscularly or transdermally. In a preferred embodiment, the invention relates to the pharmaceutical formulation for use according to the invention, wherein the pharmaceutical formulation is administered subcutaneously.

Subcutaneous, intramuscular and transdermal administration of the pharmaceutical formulation of the invention has the advantage that the pharmaceutical formulation may be self-administered by a subject. Further, the pharmaceutical formulation according to the invention may be used for the administration of subjects in which intravenous application is difficult or impossible.

In certain embodiments, the invention relates to pharmaceutical formulation for use according to the invention, wherein the pharmaceutical formulation is the enriched RPC suspension of the invention, and wherein the enriched RPC suspension is administered subcutaneously, intramuscularly, transdermally, ocullarly, such as subconjunctivally, intracamerally, intravitreally, subretinally, or suprachoroidally, to the brain, such as intralumbarly, intrathecally, or intraventricularly, intra-articularly, or by inhalation.

In other embodiments, the invention relates to pharmaceutical formulation for use according to the invention, wherein the pharmaceutical formulation is the reconstituted lyophilisate of the invention, and wherein the reconstituted lyophilisate is administered subcutaneously, intramuscularly or transdermally.

In further embodiments, the invention relates to pharmaceutical formulation for use according to the invention, wherein the pharmaceutical formulation is the re-suspended spray dried powder of the invention, and wherein the re-suspended spray dried powder is administered subcutaneously, intramuscularly, transdermally, ocullarly, such as subconjunctivally, intracamerally, intravitreally, subretinally, or suprachoroidally, to the brain, such as intralumbarly, intrathecally, or intraventricularly, intra-articularly, or by inhalation.

In certain embodiments, the invention relates to the pharmaceutical formulation according to the invention for use in the treatment of ophthalmic diseases. In certain embodiments, the ophthalmic disease may be an ocular vascular disease.

The RPCs in the pharmaceutical formulation for use in the treatment of ocular vascular diseases may comprise an antibody or an antibody fragment. In certain embodiments, the antibody or antibody fragment may specifically bind to human vascular endothelial growth factor (VEGF/VEGF-A). In certain embodiments, the antibody or antibody fragment may specifically bind to human angiopoietin-2 (Ang-2). In certain embodiments, the antibody or antibody fragment may specifically bind to VEGF/VEGF-A and Ang-2.

That is, in certain embodiments, the antibody or the antibody fragment comprised in the RPCs of the pharmaceutical formulation may be a bispecific antibody or a bispecific antibody fragment. In certain embodiments, the bispecific antibody may bind specifically to VEGF/VEGF-A and Ang-2. In certain embodiments, the bispecific anti-VEGF/anti-angiopoietin-2 (Ang-2) antibody may be faricimab, as disclosed in WO2014/009465 as “VEGFang2-0016”.

In certain embodiments, the bispecific antibody fragment may be a dutafab. In certain embodiments, the dutafab may bind specifically to VEGF/VEGF-A and/or Ang-2.

The term “ocular vascular disease” includes, but is not limited to intraocular neovascular syndromes such as diabetic retinopathy, diabetic macular edema,, retinopathy of prematurity, neovascular glaucoma, retinal vein occlusions, central retinal vein occlusions, macular degeneration, age-related macular degeneration, retinitis pigmentosa, retinal angiomatous proliferation, macular telangectasia, ischemic retinopathy, iris neovascularization, intraocular neovascularization, corneal neovascularization, retinal neovascularization, choroidal neovascularization, and retinal degeneration. (Garner, A., Vascular diseases, In: Pathobiology of ocular disease, A dynamic approach, Garner, A., and Klintworth, G.K., (eds.), 2nd edition, Marcel Dekker, New York (1994), pp. 1625-1710). As used herein, ocular vascular disorder refers to any pathological conditions characterized by altered or unregulated proliferation and invasion of new blood vessels into the structures of ocular tissues such as the retina or cornea. In one embodiment the ocular vascular disease is selected from the group consisting of: wet age-related macular degeneration (wet AMD), dry age-related macular degeneration (dry AMD), diabetic macular edema (DME), cystoid macular edema (CME), non-proliferative diabetic retinopathy (NPDR), proliferative diabetic retinopathy (PDR), cystoid macular edema, vasculitis (e.g. central retinal vein occlusion), papilledema, retinitis, conjunctivitis, uveitis, choroiditis, multifocal choroiditis, ocular histoplasmosis, blepharitis, dry eye (Sjogren’s disease) and other ophthalmic diseases wherein the eye disease or disorder is associated with ocular neovascularization, vascular leakage, and/or retinal edema. So the pharmaceutical formulation according to the invention may be useful in the prevention and treatment of wet AMD, dry AMD, CME, DME, NPDR, PDR, blepharitis, dry eye and uveitis, also preferably wet AMD, dry AMD, blepharitis, and dry eye, also preferably CME, DME, NPDR and PDR, also preferably blepharitis, and dry eye, in particular wet AMD and dry AMD, and also particularly wet AMD. In some embodiments, the ocular disease is selected from the group consisting of wet age-related macular degeneration (wet AMD), macular edema, retinal vein occlusions, retinopathy of prematurity, and diabetic retinopathy. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien’s marginal degeneration, mariginal keratolysis, rheumatoid arthritis, systemic lupus, polyarteritis, trauma, Wegeners sarcoidosis, Scleritis, Steven’s Johnson disease, periphigoid radial keratotomy, and corneal graph rejection. Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme’s disease, systemic lupus erythematosis, retinopathy of prematurity, retinitis pigmentosa, retina edema (including macular edema), Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

In certain embodiments, the pharmaceutical formulation of the invention is used for the treatment of AMD, in particular wet AMD. In certain embodiments, the pharmaceutical formulation of the invention is used for the treatment of diabetic macular edema. In certain embodiments, the RPCs comprised in the pharmaceutical formulation for the treatment of AMD and/or diabetic macular edema comprise an antibody or an antibody fragment that binds specifically to VEGF/VEGF-A and/or Ang-2. In certain embodiments, the RPCs comprised in the pharmaceutical formulation for the treatment of AMD and/or diabetic macular edema comprise a bispecific antibody that binds specifically to VEGF/VEGF-A and Ang-2. In certain embodiments, said bispecific antibody is faricimab. In certain embodiments, the RPCs comprised in the pharmaceutical formulation for the treatment of AMD and/or diabetic macular edema comprise a dutafab that binds specifically to VEGF/VEGF-A and Ang-2.

Retinopathy of prematurity (ROP) is a disease of the eye that affects prematurely born babies. It is thought to be caused by disorganized growth of retinal blood vessels which may result in scarring and retinal detachment. ROP can be mild and may resolve spontaneously, but may lead to blindness in serious cases. As such, all preterm babies are at risk for ROP, and very low birth weight is an additional risk factor. Both oxygen toxicity and relative hypoxia can contribute to the development of ROP. Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

Age-related macular degeneration begins with characteristic yellow deposits in the macula (central area of the retina which provides detailed central vision, called fovea) called drusen between the retinal pigment epithelium and the underlying choroid. Most people with these early changes (referred to as age-related maculopathy) have good vision. People with drusen can go on to develop advanced AMD. The risk is considerably higher when the drusen are large and numerous and associated with disturbance in the pigmented cell layer under the macula. Large and soft drusen are related to elevated cholesterol deposits and may respond to cholesterol lowering agents or the Rheo Procedure.

Advanced AMD, which is responsible for profound vision loss, has two forms: dry and wet. Central geographic atrophy, the dry form of advanced AMD, results from atrophy to the retinal pigment epithelial layer below the retina, which causes vision loss through loss of photoreceptors (rods and cones) in the central part of the eye. While no treatment is available for this condition, vitamin supplements with high doses of antioxidants, lutein and zeaxanthin, have been demonstrated by the National Eye Institute and others to slow the progression of dry macular degeneration and in some patients, improve visual acuity.

Retinitis pigmentosa (RP) is a group of genetic eye conditions. In the progression of symptoms for RP, night blindness generally precedes tunnel vision by years or even decades. Many people with RP do not become legally blind until their 40s or 50s and retain some sight all their life. Others go completely blind from RP, in some cases as early as childhood. Progression of RP is different in each case. RP is a type of hereditary retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease.

Macular edema occurs when fluid and protein deposits collect on or under the macula of the eye, a yellow central area of the retina, causing it to thicken and swell. The swelling may distort a person’s central vision, as the macula is near the center of the retina at the back of the eyeball. This area holds tightly packed cones that provide sharp, clear central vision to enable a person to see form, color, and detail that is directly in the line of sight. Cystoid macular edema is a type of macular edema that includes cyst formation.

In certain embodiments the pharmaceutical formulation according to the invention is administered alone (without an additional therapeutic agent) for the treatment of one or more ocular vascular diseases described herein.

In other embodiments the pharmaceutical formulation according to the invention may be administered in combination with one or more additional therapeutic agents or methods for the treatment of one or more ocular vascular diseases described herein.

The additional therapeutic agents may include, but are not limited to, Tryptophanyl-tRNA synthetase (TrpRS), Eye001 (Anti-VEGF Pegylated Aptamer), squalamine, RETAANE(TM) (anecortave acetate for depot suspension; Alcon, Inc.), Combretastatin A4 Prodrug (CA4P), MACUGEN(TM), MIFEPREX(TM) (mifepristone-ru486), subtenon triamcinolone acetonide, intravitreal crystalline triamcinolone acetonide, Prinomastat (AG3340-synthetic matrix metalloproteinase inhibitor, Pfizer), fluocinolone acetonide (including fluocinolone intraocular implant, Bausch & Lomb/Control Delivery Systems), VEGFR inhibitors (Sugen), VEGF-Trap (Regeneron/Aventis), VEGF receptor tyrosine kinase inhibitors such as 4-(4-bromo-2-fluoroanilino)-6-methoxy-7-(l-methylpiperidin-4-ylmethoxy)quinazoline (ZD6474), 4-(4-fluoro-2-methylindol-5-yloxy)-6-methoxy-7-(3-pyrrolidin-1-ylpropoxy)quinazoline (AZD2171), vatalanib (PTK787) and SU1 1248 (sunitinib), linomide, and inhibitors of integrin v.beta.3 function and angiostatin.

Other pharmaceutical therapies that may be used in combination with the pharmaceutical formulation according to the invention include, but are not limited to, VISUDYNE(TM) with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEtema Laboratories, Inc.), Sima-027 (Sima Therapeutics, Inc.), pegaptanib (NeXstar Pharmaceuticals/Gilead Sciences), neurotrophins (including, by way of example only, NT-⅘, Genentech), Cand5 (Acuity Pharmaceuticals), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/ Albany, Mera Pharmaceuticals), D-9120 (Celltech Group pic), ATX-S10 (Hamamatsu Photonics), TGF-beta 2 (Genzyme/Celtrix), tyrosine kinase inhibitors (Allergan, SUGEN, Pfizer), NX-278- L (NeXstar Pharmaceuticals/Gilead Sciences), Opt-24 (OPTIS France SA), retinal cell ganglion neuroprotectants (Cogent Neurosciences), N— nitropyrazole derivatives (Texas A&M University System), KP-102 (Krenitsky Pharmaceuticals), cyclosporin A, Timited retinal translocation”, photodynamic therapy, (including, by way of example only, receptor-targeted PDT, Bristol-Myers Squibb, Co.; porfimer sodium for injection with PDT; verteporfin, QLT Inc.; rostaporfm with PDT, Miravent Medical Technologies; talaporfm sodium with PDT, Nippon Petroleum; motexafm lutetium, Pharmacyclics, Inc.), antisense oligonucleotides (including, by way of example, products tested by Novagali Pharma SA and ISIS- 13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

Any anti-angiogenic agent may be used in combination with the pharmaceutical formulation according to the invention, include, but are not limited to those listed by Carmeliet and Jain, 2000, Nature 407:249-257. In certain embodiments, the anti-angiogenic agent may be a VEGF antagonist or a VEGF receptor antagonist such as VEGF variants, soluble VEGF receptor fragments, aptamers capable of blocking VEGF or VEGFR, neutralizing anti- VEGFR antibodies, low molecule weight inhibitors of VEGFR tyrosine kinases and any combinations thereof and these include anti-VEGF aptamers (e.g. Pegaptanib), soluble recombinant decoy receptors (e.g. VEGF Trap).. In certain embodiments, the anti-angiogenic agent may include corticosteroids, angiostatic steroids, anecortave acetate, angiostatin, endostatin, small interfering RNA’s decreasing expression of VEGFR or VEGF ligand, post- VEGFR blockade with tyrosine kinase inhibitors, MMP inhibitors, IGFBP3, SDF-1 blockers, PEDF, gamma-secretase, Delta-like ligand 4, integrin antagonists, HIF-1 alpha blockade, protein kinase CK2 blockade, and inhibition of stem cell (i.e. endothelial progenitor cell) homing to the site of neovascularization using vascular endothelial cadherin (CD- 144) and stromal derived factor (SDF)-I antibodies. Small molecule RTK inhibitors targeting VEGF receptors including PTK787 can also be used. Agents that have activity against neovascularization that are not necessarily anti-VEGF compounds can also be used and include anti-inflammatory drugs, m-Tor inhibitors, rapamycin, everolismus, temsirolismus, cyclospohne, anti-TNF agents, anti-complement agents, and nonsteroidal antiinflammatory agents. Agents that are neuroprotective and can potentially reduce the progression of dry macular degeneration can also be used, such as the class of drugs called the ‘neurosteroids.’ These include drugs such as dehydroepiandrosterone (DHEA)(Brand names: Prastera(R) and Fidelin(R)), dehydroepiandrosterone sulfate, and pregnenolone sulfate. Any AMD (age-related macular degeneration) therapeutic agent can be used in combination with the pharmaceutical formulation according to the invention, including but not limited to verteporfin in combination with PDT, pegaptanib sodium, zinc, or an antioxidant(s), alone or in any combination.

In embodiments where the pharmaceutical formulation according to the invention is used for the treatment of ophthalmic diseases, such as, without limitation, ocular vascular diseases, it is preferred that the pharmaceutical formulation according to the invention is administered intraocularly or intravitreally.

The term “intraocular” as used herein refers to anywhere within the globe of the eye. The term “intravitreal” as used herein refers to inside the gel in the back of the eye. That is, the pharmaceutical formulation for the treatment of ophthalmic diseases may be administered subretinally, intracapsularly, suprachoroidally, intracamerally, intrapalpebrally, to the subtenon, to the subconjunctival area, to the cul-de-sac, to the retrobulbar space or to the pribulbar space.

The term “subretinal” as used herein refers to the area between the retina and choroid. The term “intracapsular” as used herein refers to within the lens capsule. The term “suprachoroidal” as used herein refers to the area between the choroid and sclera. The term “subtenon” as used herein refers to the area posterior to the orbital septum, outside the sclera, below tenon’s capsule. The term “subconjunctival” as used herein refers to the area between the conjunctiva and sclera. The term “intracameral” as used herein refers to “into a chamber” of the eye, for e.g., into the anterior or posterior chamber of the eye. The term “intrapalpebral” as used herein refers to into the eyelid. The term “cul-de-sac” as used herein refers to the space between the eyelid and globe. The term “retrobulbar” as used herein refers to behind the orbit of the eye. The term “peribulbar” as used herein refers to within the orbit or adjacent to the eye.

In another aspect, the invention relates to the use of the pharmaceutical formulation according to the invention for the treatment of a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis.

In another aspect, the invention relates to the use of the pharmaceutical formulation according to the invention in the preparation of a medicament for treatment of a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis.

In certain embodiments, the invention relates to the use of the pharmaceutical formulation according to the invention for the treatment of ophthalmic diseases. In certain embodiments, the invention relates to the use of the pharmaceutical formulation according to the invention in the preparation of a medicament for the treatment of ophthalmic diseases. The ocular disease may be any one of the ophthalmic diseases disclosed herein.

In another embodiment, the invention relates to a method of treating a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis, the method comprising the steps of (a) producing a pharmaceutical formulation according to the method of the invention; and (b) administering the pharmaceutical formulation to a subject in need thereof.

In a preferred embodiment, the invention relates to a method of treating a subject suffering from a disease selected from the group consisting of: autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis, the method comprising the steps of (a) producing a pharmaceutical formulation according to the method of the invention; and (b) administering the pharmaceutical formulation to a subject in need thereof, wherein the pharmaceutical composition is administered subcutaneously, intramuscularly or transdermally, in particular wherein the pharmaceutical composition is administered subcutaneously.

In another embodiment, the invention relates to a method of treating an ophthalmic disease, in particular an ocular vascular disease, the method comprising the steps of (a) producing a pharmaceutical formulation according to the method of the invention; and (b) administering the pharmaceutical formulation to a subject in need thereof. In certain embodiments, the pharmaceutical formulation for use in the method of treating an ophthalmic disease may comprise an antibody or antibody fragment that specifically binds to VEGF/VEGF-A and Ang-2. In certain embodiments, the pharmaceutical formulation for use in the method of treating an ophthalmic disease may be administered intraocularly or intravitreally.

In another embodiment, the invention relates to a method of subcutaneous, intramuscular or transdermal administration of a pharmaceutical formulation, the method comprising the steps of (a) producing a pharmaceutical formulation according to the method of the invention; and (b) administering the pharmaceutical formulation to a subject subcutaneously, intramuscularly, transdermally.

In another embodiment, the invention relates to a method of ocularly administering a pharmaceutical formulation to a subject, the method comprising the steps of (a) producing a pharmaceutical formulation according to the method of the invention; and (b) administering the pharmaceutical formulation to a subject ocularly, such as subconjunctivally, intracamerally, intravitreally, subretinally, or suprachoroidally.

In another embodiment, the invention relates to a method of administering a pharmaceutical formulation to the brain of a subject, the method comprising the steps of (a) producing a pharmaceutical formulation according to the method of the invention; and (b) administering the pharmaceutical formulation to the brain of a subject, such as intralumbarly, intrathecally, or intraventricularly.

In another embodiment, the invention relates to a method of intra-articularly administering a pharmaceutical formulation to a subject, the method comprising the steps of (a) producing a pharmaceutical formulation according to the method of the invention; and (b) administering the pharmaceutical formulation to a subject intra-articularly.

The term “administration”, as used herein to refer to the delivery of an inventive pharmaceutical formulation to a subject, is not limited to any particular route but rather refers to any route accepted as appropriate by the medical community. For example, the present invention contemplates routes of delivering or administering that include, but are not limited to, subcutaneously, intramuscularly, transdermally, ocullarly, such as subconjunctivally, intracamerally, intravitreally, subretinally, or suprachoroidally, to the brain, such as intralumbarly, intrathecally, or intraventricularly, intra-articularly, or by inhalation.. In certain embodiments of the invention, administration is subcutaneously.

The term “subcutaneous,” as used herein, refers to below the skin (e.g., in the connective tissue underlying the dermis and above the facia of the muscle tissue).

The term “intramuscular” as used herein refers to the intramuscular route (IM route). In the IM route of administration, injections are made into the striated muscle fibers that lie beneath the subcutaneous layer.

The term “transdermal” as used herein means passage into and/or through skin or mucosa for localized or systemic delivery of an active agent.

Within the present invention, it is preferred that the pharmaceutical formulation according to the invention is administered to the subject in a therapeutically effective amount.

As used herein, the term “therapeutically effective amount” means an amount that is sufficient, when administered to an individual suffering from or susceptible to a disease, disorder, and/or condition, to treat the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response when administered or delivered to a significant number of subjects in need of such treatment. It is specifically understood that particular subjects may, in fact, be “refractory” to a “therapeutically effective amount.” It is to be understood that he therapeutically effective amount may differ between pharmaceutical formulations comprising different proteins. However, the skilled person is aware of methods to determine the amount of the composition that is required to obtain the desired therapeutic effect.

In certain embodiments, the pharmaceutical formulation according to the invention is administered to a subject subcutaneously, intramuscularly, transdermally, ocullarly, such as subconjunctivally, intracamerally, intravitreally, subretinally, or suprachoroidally, to the brain, such as intralumbarly, intrathecally, or intraventricularly, intra-articularly, as a liquid composition with a protein concentration of at least 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL or 200 mg/mL. The volume of the pharmaceutical formulation administered as a single dose may range from 0.1 to 10 mL, 0.1 to 9 mL, 0.1 to 8 mL, 0.1 to 7 mL, 0.1 to 6 mL, 0.1 to 5 mL, 0,1 to 4 mL, 0.1 to 3 mL, 0.1 to 2 mL, 0.1 to 1 mL, 1 to 3 mL, or 1 to 2 mL.

The term “subject” or “patient,” as used herein, refers to any animal to which the pharmaceutical formulation according to the invention may be delivered or administered. For example, a subject may be a human, dog, cat, cow, pig, horse, mouse, rat, gerbil, hamster etc. In many embodiments of the present invention, the subject is a human.

As used herein, the term “treatment” (also “treat” or “treating”) refers to any administration of a biologically active agent that partially or completely alleviates, ameliorates, relives, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

While aspects of the invention are illustrated and described in detail in the Figures, Tables and in the foregoing description, such Figures, tables and description are to be considered illustrative or exemplary and not restrictive. Also reference signs in the claims should not be construed as limiting the scope.

It will also be understood that changes and modifications may be made by those of ordinary skill within the scope and spirit of the claims. In particular, the present invention covers further embodiments with any combination of features from different embodiments described above. It is also to be noted in this context that the invention covers all further features shown in the figures individually, although they may not have been described in the previous or following description. Also, single alternatives of the embodiments described in the figures and the description and single alternatives of features thereof can be disclaimed from the subject matter according to aspects of the invention.

Whenever the word “comprising” is used in the claims, it should not be construed to exclude other elements or steps. It should also be understood that the terms “essentially”, “substantially”, “about”, “approximately” and the like used in connection with an attribute or a value may define the attribute or the value in an exact manner in the context of the present disclosure. The terms “essentially”, “substantially”, “about”, “approximately” and the like could thus also be omitted when referring to the respective attribute or value. The terms “essentially”, “substantially”, “about”, “approximately” when used with a value may mean the value ±10%, preferably ±5%.

A number of documents including patent applications, manufacturer’s manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### BRIEF DISCRIPION OF FIGURES

FIG. 1: Visual aspect of RPC suspension obtained after mixing VEGF-Ang2 and dextran sulfate solutions at 1:1 mole-charge ratio in histidine buffer 20 mM pH 5.0.

FIG. 2: Protein melting temperature before (naked protein) and after complexation (RPC) and dissociation.

FIG. 3: DSC thermogram showing the melting temperature of a, VA2 in a complexed form (Tm=144.2° C.) and b, dextran sulfate sodium salt (Tm=171.9° C.) present in RPC formulations.

FIG. 4: Visual aspect of RPC suspension at 200 mg/mL showing the paste-like aspect of the formulation. a, spatula kept face up; b, spatula turned face down.

FIG. 5: Visual aspect of spray dried VA2 RPC.

FIG. 6: SEM images of VA2 RPC particles after spray drying of the suspensions in histidine buffer and in Ultra pure water (MilliQ water).

FIG. 7: VA2 RPC spray dried powder suspended in NAS solvents. EO, ethyl oleate; IDME, isosorbide dimethyl ether.

FIG. 8: Percentages of complexation and dissociation of the different protein formats with dextran sulfate (DS) at 1:1 mole-charge ratio.

FIG. 9 : Percentage complexation and dissociation of RPC using VEGF-Ang2 as a protein model and different complexing agents.* Complexation performed at pH 4.0. ** Dissociation performed with PBS 100 mM.

FIG. 10: Percentages of complexation and dissociation of VA2 and DS at different protein concentrations.

FIG. 11: RPC particle size obtained after complexation with DS at different protein concentrations.

FIG. 12: Percentage complexation of VA2 and DS in histidine buffer 20 mM at different pH; and their corresponding percentage dissociation in PBS.

FIG. 13: Percentage complexation of VA2 and DS in histidine buffer pH 5.0 at different ionic strengths; and their corresponding percentage dissociation in PBS.

FIG. 14: Percentage complexation and dissociation of VA2 with DS in presence of different additives. FIG. 15: Visual aspect of the VA2 RPC in a, histidine buffer (control); b, in presence of different additives (sucrose, polysobate 20, poloxamer 188 or mixture of those); c, in ultra pure water (MilliQ water) (no complexation).

FIG. 16: Visual aspect of RPC formulations (F1-F4) and VA2 control DP solution after 4 weeks storage at different temperatures. Note hard-cake formation, gel-aspect and shrinkage of RPC suspensions after 4 weeks at 25 or 40° C. A, front view; B, bottom view.

FIG. 17: Visual aspect of VA2 RPC 60 mg/mL a, before lyophilization; b and c, after lyophilization (b, front view; c, bottom view); d, after reconstitution of the lyophilized cake in PBS (120 mg/mL) stored 4-weeks at 5° C.

FIG. 18: Visual aspect of RPC formulation after 4 weeks storage at different temperatures. Ctrl - control (in histidine buffer, no buffer exchange), C — centrifuged (in MilliQ, buffer exchange by centrifugation), D - dialyzed (in MilliQ, buffer exchange by dialysis), A - front view, B - bottom view before vortexing, C — bottom view after vortexing.

FIG. 19: Comparison of visual aspect of the samples - “hard-cake” has formed in control sample (left), whereas particles in dialysed sample remained dispersed (right).

FIGS. 20A-20B: Comparison of particle size for RPC that had buffer exchanged to ultrapure water by means of centrifugation (FIG. 20A) or dialysis (FIG. 20B). The first results were obtained by laser diffraction measuring techniques (FIG. 20A), whereas the second by dynamic light scattering (FIG. 20B).

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1: Development of Reversible Protein Complex (RPC) Formulations

### 1.1 Materials

Therapeutic proteins including monoclonal antibodies (mAbs), bispecific crossmAb, cytokine fusion mAb and DutaFab, were provided by F.Hoffmann-La Roche AG (Basel, Switzerland). Histidine-HCl and L-Histidine base were obtained from Ajinomoto (Osaka, Japan), citric acid and Trisodium citrate from Merck (Darmstadt, Germany), polysorbate 20 from Croda (East Yorkshire, UK), poloxamer 188 from BASF (Ludwigshafen, Germany) and sucrose from Pfanstiehl (Zug, Switzerland). Dextran sulfate sodium salt (DS), Sodium dodecyl sulfate (SDS), Chondroitin sulfate (CS), Sodium taurocholate hydrate (ST), Triacetin, Diethylene glycol monoethyl ether (Transcutol®), Isosorbide dimethyl ether, Tetraglycol (Glycofurol), Ethyl oleate, PBS tablets and PVDF filters were purchased from Sigma-Aldrich (Buchs, Switzerland). Slide-A-Lyzer Dialysis Cassettes (MWCO 10 K) were obtained from Thermo Scientific. MilliQ water (resistivity > 18 MQ cm) was prepared using a Merck Millipore MilliQ water purification system (Darmstadt, Germany). All solvents used were from an analytical grade.

### 1.2 Methods

### 1.2.1 Preparation of Protein Solutions and Complexing Agent Solutions

The different protein solutions in histidine buffer (20 mM, pH 5.3-5.8), containing mainly sucrose, surfactants and/or sodium chloride; were dialyzed prior complexation to exchange the buffer with fresh histidine buffer (20 mM, pH 5.0). Dialysis was run during 2h at room temperature then over night at 5 ± 3° C. The dialyzed protein was further diluted to 5 mg/mL in histidine buffer (20 mM, pH 5.0).

Complexing agent solutions (Dextran sulfate sodium salt, Sodium dodecyl sulfate, Chondroitin sulfate, Sodium taurocholate hydrate) were prepared at 50 mg/mL in histidine buffer (20 mM, pH 5.0).

### 1.2.2 Protein Content

Protein concentration was measured by UV absorbance at 280 nm using a spectrophotometer (NanoDrop One^{c}, ThermoFisher Scientific). In order to determine the protein concentration, RPC suspension was first dissociated using PBS 10 mM, then 4 µL were placed on the instrument pedestal for quantification.

### 1.2.3 Formation of Reversible Protein Complexes

Proteins charge was calculated from their amino acid sequences, then the corresponding charge per mole was determined for each protein. The mole-charge was also determined for each of the complexing agents. Reversible protein complexes were prepared by mixing the protein solution (5 mg/mL) with the complexing agent solution (50 mg/mL) at 1:1 mole-charge ratio, aiming for a 100% charge neutralization. Total neutralization of the protein charge by the complexing agent leads to precipitation of the protein and formation of a whitish protein-particulate suspension.

Percentage complexation was determined after centrifugation of 1 mL sample of the RPC suspension (10 000 rpm, 5 min) and quantifying the amount of protein remaining in the supernatant using UV spectrometry (nanoDrop One^{c}, Thermo Scientific) according to the following equation:

$$C o m p l e x a t i o n \left(\%\right) = \frac{i n i t i a l p r o t . c o n c - P r o t . c o n c . i n s u p e r n a t a n t}{I n i t i a l p r o t e i n c o n c e n t r a t i o n}$$

### 1.2.4 Dissociation of Reversible Protein Complexes

Reversible protein complexes were dissociated following pH increase by diluting the RPC suspension to 1 mg/mL final protein concentration in PBS 10 mM, pH 7.4. Proteins being uncharged at pH 7.4, charge interactions between the proteins and the complexing agents decrease leading to dissociation of the complexes.

Protein concentration following dissociation was determined by UV and the percentage dissociation was calculated according the following equation:

$$D i s s o c i a t i o n \left(\%\right) = \frac{c o n c . p r o t e i n d i s s o c i a t e d}{c o n c . p r o t e i n c o m p l e x e d}$$

### 1.2.5 Particle Size

RPC particle size distribution was measured using a laser diffraction analyzer (Partica LA-960, HORIBA). RPC suspension was loaded in the sample bath of the instrument containing Ultra pure water (MilliQ water) to a concentration that allows 70-95% transmittance then the measurement was performed under circulation mode. Mean particle size values are reported. Particle size distribution of the spray dried RPC powder was also evaluated using SEM.

### 1.2.6 Zeta-Potential

Particle surface charge was evaluated using Malvern Zetasizer Nano ZS. Particle surface charge of RPC was evaluated in both histidine buffer and Ultra pure water (MilliQ water) media. Since complexation only occurs under specific conditions, complexation was first performed in histidine buffer then dialysis was run against Ultra pure water (MilliQ water) to remove buffer ions. RPC suspensions in both media were diluted to 0.1 mg/mL in their corresponding media then samples (~0.8 mL) were loaded into the zeta potential cells for analysis.

### 1.2.7 Viscosity

Viscosity measurements were performed using a rheometer (Physica MCR 301, Anton Paar) equipped with a cone-plate geometry. The viscosity of RPC formulations was determined by placing 80 µL of the sample in the center of the plate. The method used consisted of 3 steps; 120 s for sample equilibration to 20° C. in the first step, followed by a second step where 1000 s⁻¹ shear rate was applied for 10 s, then a last step where 1000 s⁻¹ shear rate was applied for 5 s.

### 1.2.8 Protein Stability

Protein stability was evaluated before complexation and after complexation and dissociation. Protein purity was monitored using size exclusion chromatography (SEC) and protein charge was monitored using ion exchange chromatography (IEC).

### 1.2.9 Protein Melting Temperature

Melting temperature of reversible protein complexes was measured in both liquid form (suspension) using nanoDSF and solid form (spray-dried powder) by DSC.

### 1.2.9.1 Melting Temperature by nanoDSF

Protein melting temperature was measured before complexation as naked protein, after complexation as RPC suspension, and after dissociation of RPC in PBS 10 mM. 30 µL of each solution (~ 1.2 mg/mL) were placed in a 384 well microplate, transferred into a 48 capillary array (~10 µL) then introduced in the instrument (Prometheus, nanoTemper). The method consists on applying a temperature ramp from 25 to 95° C. with a 0.5° C./min heating rate. Excitation power was set at 28%, protein fluorescence intensities were recorded (ratio 350/330 nm) and melting temperature (transition midpoint Tₘ, 50% unfolded protein) was recorded.

### 1.2.9.2 Melting Temperature by DSC

Protein melting temperature as RPC powder was measured after complexation and spray drying using DSC (Q2000, TA instruments). The sample was placed in the instrument (~ 50 mg) then the method consisted of an equilibration step at 25° C. followed by a ramp of 5° C./min to -5° C. and an isothermal step for 5 min, then a ramp of 2° C./min to 250° C. and an isothermal step for 5 min and finally a ramp of 5° C./min to 25° C.

### 1.3 Results

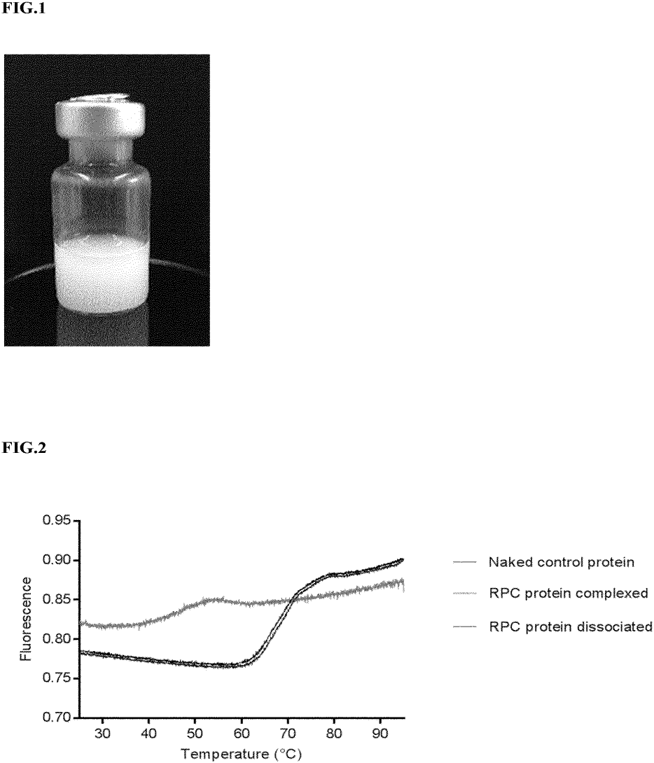

Mixing the protein (VEGF-Ang2) solution with the complexing agent (dextran sulfate) solution in histidine buffer (20 mM pH 5.5) at 1:1 mole-charge ratio resulted in the formation of a whitish suspension of protein particles (FIG. 1)

Mean particle size of the RPC suspension measured by laser diffraction was 10.8 ± 0.8 µm. Particles sedimentation was observed when formulation was left to stand, however, particles were easily resuspended after simple agitation.

Surface charge of the RPC particles in histidine buffer was -13.9±0.6 mV; after buffer exchange (wash) with Ultra pure water (MilliQ water) using dialysis, the surface charge was -43.6±0.5 mV.

Stability of the protein evaluated by SEC showed no significant difference before and after complexation and dissociation in PBS with percentages monomer of 95.2% and 95.3%, respectively. Same observation using IEC with percentages main peak of 67.2% and 66.7%, respectively before and after complexation and dissociation in PBS.

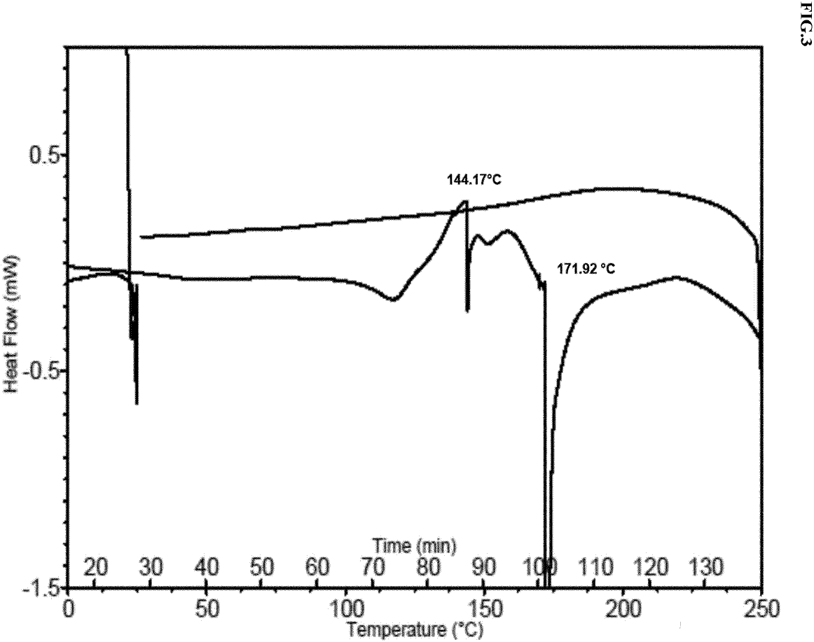

Protein melting temperature (Tm) was determined in the control naked form (uncomplexed), after complexation as a suspension form and after dissociation of RPC in PBS. Results showed no significant difference in the Tm of the naked control protein (67.5° C.) and after complexation and dissociation of the RPC (68.4° C.) confirming the results observed by SEC and IEC concluding that complexation-dissociation process does not affect the stability of the protein (FIG. 2). Interestingly, the complexed protein (RPC) does not show a clear inflection point suggesting that the Tm of the protein in the complexed form shifted to temperatures higher than 95° C. (limit of the nanoDSF temperature range), which indicates a higher stability compared to the naked protein.

DSC was further used to determine the exact Tm of the protein in the complexed form (FIG. 3). Results showed a shift of the protein Tm from 67.5° C. to 144.2° C. when it is complexed with DS, demonstrating a higher stability of the protein in the RPC form.

### Example 2: Post-Processing Steps of Reversible Protein Complex Formulations

RPC concept was used to develop highly concentrated protein formulations. Two approaches were evaluated; the first consists on up-concentration of the RPC suspension in histidine buffer to a series of concentrations ranging from 60 mg/mL up to 200 mg/mL. The second approach consists on re-suspending the spray dried RPC particles in a non-aqueous solvent.

### 2.1 Up-Concentration

RPC suspensions were up-concentrated by centrifugation (Eppendorf Centrifuge 5810 R). RPC suspension (5 mg/mL) was filled into falcon tubes (50 mL) containing magnetic stirrers then centrifuged (3900 rpm, 15 min, 5° C.) for up-concentration. Supernatant was discarded (100% complexation) then depot were homogenized and pooled into one falcon tube. Intermediate concentration was determined by dissociating 10 µL suspension in 990 µL PBS 10 mM pH 7.4. Concentration was adjusted to 120 mg/mL by further centrifugation or dilution with histidine buffer.

For higher concentrations (up to 200 mg/mL), high-speed centrifuge (Beckman Coulter Optima L-90K Prep Ultracentrifuge) was used for up-concentration. RPC suspension was placed in a high-speed centrifuge tube (Beckman Coulter, 70 mL) containing magnetic stirrer then centrifuged at 10 000 rpm during 10 min at 5° C. Supernatant was removed then depot was homogenized by vortex, 10 µL were dissociated in 990 µL PBS to evaluate the intermediate concentration. Centrifugation was carried on until reaching final target concentration of 200 mg/mL.

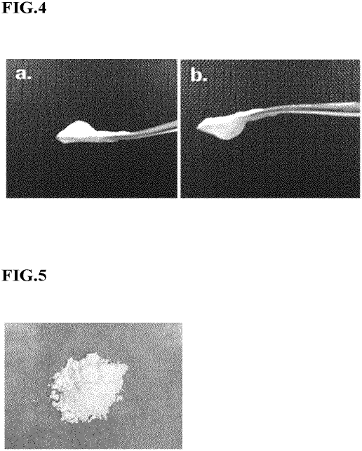

Up-concentration of RPC suspension up to 200 mg/mL resulted in a very dense white suspension with a paste-like aspect (FIG. 4). Nevertheless, this formulation was injectable through a 26G needle. RPC suspensions at 180 and 160 mg/mL had a similar paste-like aspect, whereas RPC suspensions become more liquid at around 120 mg/mL, and RPC suspensions at 90 and 60 mg/mL were liquid.

Viscosity measurement of the different up-concentrated suspensions showed a shear thinning effect (viscosity decrease upon application of a constant shear rate). This feature renders the injectability of the formulation easier compared to a Newtonian solution. However, the viscosity measurement method used was developed for liquid solutions and was not appropriate for the paste-like RPC formulation.

### 2.2 Spray Drying

Spray drying was performed using Büchi Mini Spray Dryer B-290. Inlet temperature was set at 115° C. (outlet ~ 48° C.) and nitrogen aspiration was set at 100%. The feed rate (peristaltic pump) was set at 17 mL/min. RPC formulation was kept under stirring to avoid particle sedimentation during spray drying process.

Spray drying of VA2 RPC formulations at 5 mg/mL (F1-F3) resulted in a fine, white powder (FIG. 5).

In order to optimize the protein content in the spray dried RPC powder and the stability of the protein following the spray drying process of RPC suspensions, different formulation compositions (F1 to F3) were evaluated (Table 1). RPC formulations were prepared as mentioned in Example 1, then the corresponding amounts of sucrose and polysorbate 20 were added to F1. Since the inventors targeted the highest protein content in the final powder, the inventors used the minimal excipients possible to limit their contribution to the final powder content while still ensuring protein stability during spray drying process. Given that the components of histidine buffer highly contribute to the final solid content of the RPC powder, histidine buffer was exchanged with ultra pure water (MilliQ water) using dialysis following complexation (F2 and F3). The corresponding amounts of sucrose and polysorbate 20 were then added to F3.

TABLE 1

Composition of RPC solutions at 5 mg/mL to be spray dried.

| Excipient | Medium | Sucrose (mg/mL) | Polysorbate 20 (mg/mL) |
|---|---|---|---|
| F1 | Histidine buffer | 2.05 | 0.40 |
| F2 | Ultra pure water (MilliQ water) | - | - |
| F3 | Ultra pure water (MilliQ water) | 1.00 | 0.20 |

Following spray drying, protein content in the RPC powder was determined by UV after dissociation of a specific amount in PBS, and using the following the equation:

$$P r o t e i n c o n t e n t \left(\%\right) = \frac{A c t u a l V A 2 c o n c .}{T h e o r i t i c a l V A 2 c o n c .}$$

Protein stability was evaluated by SEC and IEC and protein melting temperature was determined by DSC in RPC as powder form and by nanoDSF after solubilisation in PBS.

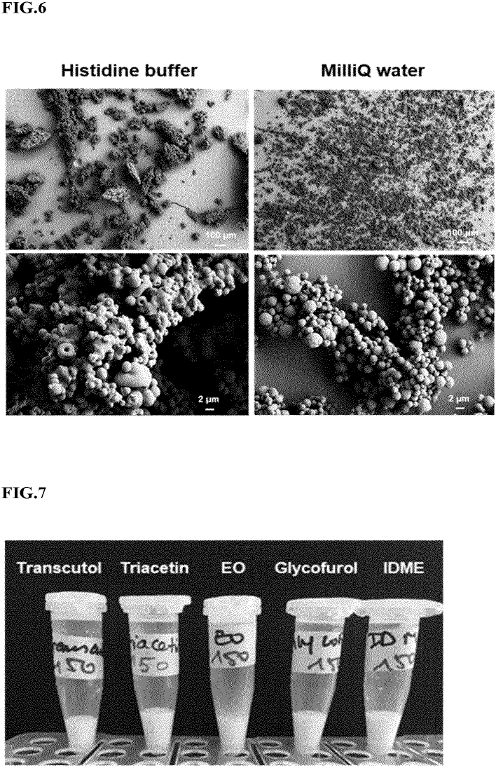

Scanning electron microscope (SEM) analysis of F1 and F3 particles obtained after spray drying showed loose, round and dispersed particles in VA2 RPC washed with Ultra pure water (MilliQ water) (F3, FIG. 6), while particles in VA2 RPC in histidine buffer were more agglomerated forming clusters and fused-like particles (F1, FIG. 6). Whether this difference in particle shape is due to the different media used or to the spray drying process itself is to be further investigated.

Analysis of VA2 RPC particles in histidine buffer versus Ultra pure water (MilliQ water) before and after spray drying showed an increase in the particle size after spray drying, from ~8 µm to ~30 µm (Table 2), probably due to the adsorption of the excipients added (sucrose and PS20) on the RPC particles and possible cluster formation during the spray drying process.

TABLE 2

VA2 RPC particle size in histidine buffer versus Ultra pure water (MilliQ water) before and after spray drying.

| Formulation | Mean (µm) | Median (µm) |
|---|---|---|
| F1 -Before SD | 7.2 | 6.4 |
| F1 - After SD | 30.6 | 20.1 |
| F3 - Before SD | 8.7 | 7.3 |
| F3 - After SD | 33.5 | 8.2 |

As expected, protein content in the spray dried powder varied in the formulations (F1-F3) according to the amount of the buffer salts and the excipients added (Table 3).

TABLE 3

Protein content and stability in VA2 RPC spray dried powder.

| VA2:DS | Medium | Excipients | VA2 cont. (%) | SEC (% mono) | IEC (% main peak) |
|---|---|---|---|---|---|
| F1 1:1 | Histidine buffer 20 mM pH 5.0 | DS 0.84 mg/mL Sucrose 2.1 mg/mL PS20 0.4 mg/mL | 43.1 | 97.1 | 65.2 |
| F2 1:1 | Ultra pure water (MilliQ water) | DS 0.84 mg/mL | 70.2 | 94.6 | 66.8 |
| F3 1:0.6 | Ultra pure water (MilliQ water) | DS 0.56 mg/mL Sucrose 1 mg/mL PS20 0.2 mg/mL | 63.0 | 95.5 | 66.1 |

Protein content in F1 was 43.1%, with a high contribution of histidine buffer salts to the solid content of the spray dried powder. Removal of the excipient and washing out histidine buffer with MQ water in F2 increased the protein content up to 70.2%. Decreasing the protein:DS mole-charge ratio to 1:0.6, followed by a wash out of the histidine buffer salts and a decrease of the amount of excipients added in F3 led to a final protein content of 63.0%.

Protein stability during spray drying process was assessed by SEC and IEC. No significant change was observed in the chemical stability (IEC) in all formulations. However, an increase in the high molecular weight species (HMWS) was observed by SEC, especially in F2, probably due to the absence of histidine buffer and excipients to protect the RPC during the spray drying process. Indeed, in F3, adding half excipients could decrease the HMW species. Hence, F3 seems to be a good compromise to optimize the protein content in the final spray dried powder and ensure the protein stability during the spray drying process.

### 2.3 Resuspension of Spray Dried RPCs

A series of apolar solvents were tested as resuspension media including ethyl oleate, triacetin, Transcutol (diethylene glycol monoethyl ether), Glycofurol, and Isosorbide dimethyl ether. RPC spray dried powder was incorporated in the corresponding volume of the solvent to reach 100, 150, 200 and 250 mg/mL then homogenized.

RPC dissociation after up-concentration or resuspension in non-aqueous media was evaluated after dilution in PBS. Melting temperature of the spray dried RPC powder was evaluated by DSC and using nanoDSF after dissociation in PBS. Protein stability after up-concentration or resuspension in non-aqueous media was evaluated by SEC and IEC. Particle size and viscosity of the high-concentration formulations were evaluated as mentioned in Example 1.

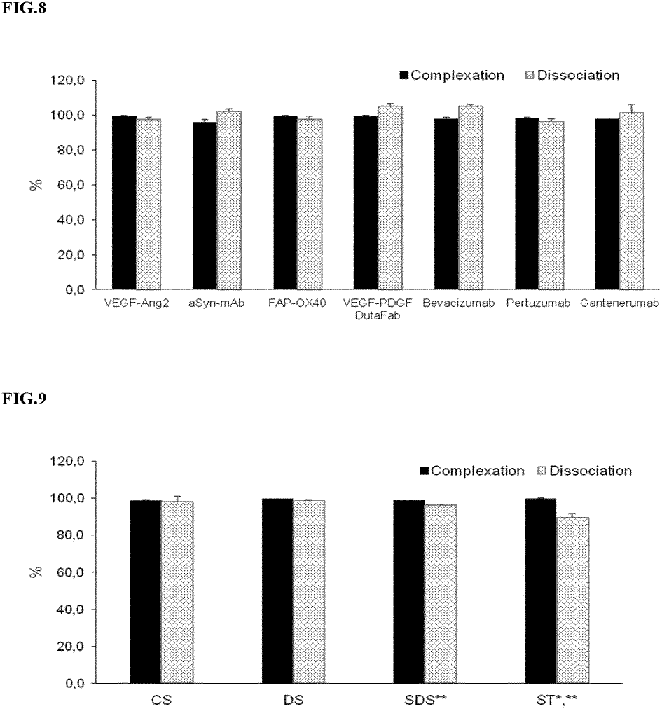

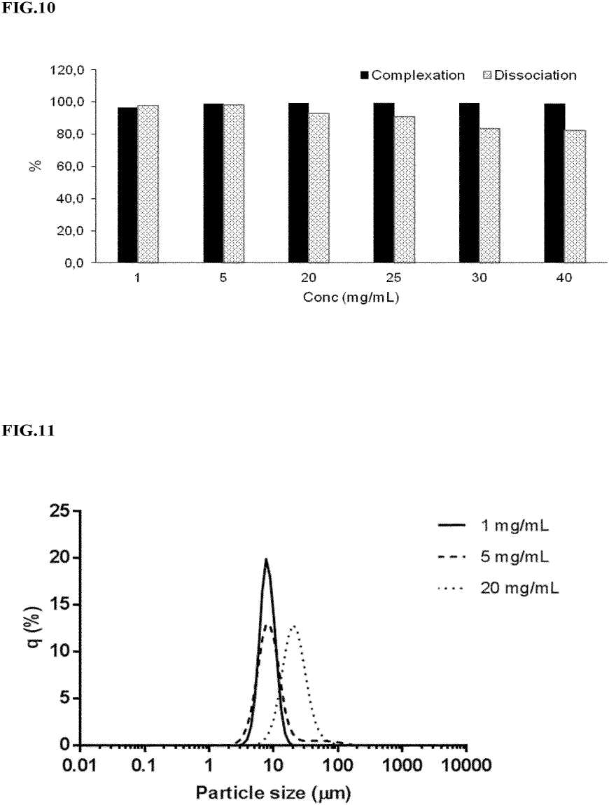

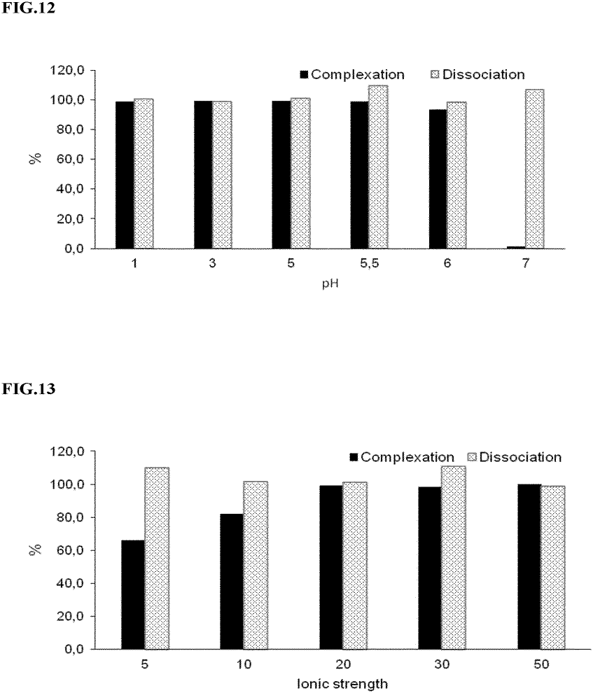

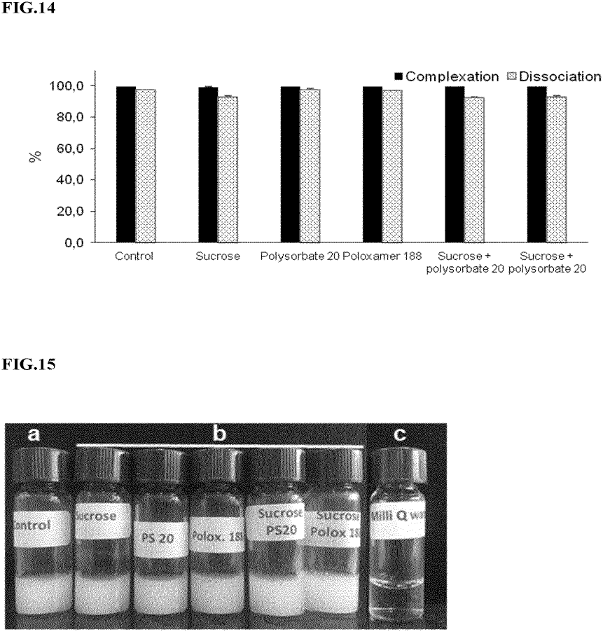

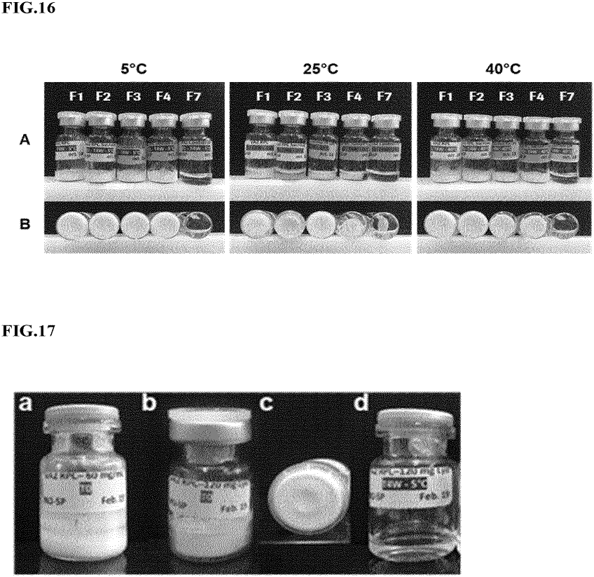

Suspension of VA2 RPC spray dried powder in NAS resulted in white suspensions (FIG. 7).

After dissolving 110 mg of RPC spray dried powder in 0.25 mL PBS, the final volume was 0.29 mL. The contribution of the solid content is 0.04 mL (~14% of the total volume). This volume was taken into account when preparing the VA2 RPC-NAS suspensions at different concentrations (ex. spray dried powder was dissolved in 0.26 mL NAS for a total volume of 0.3 mL). The target and actual concentrations of VA2 in NAS are listed in Table 4.

TABLE 4

Target and actual protein concentrations in NAS, their corresponding viscosity and stability (SEC and IEC).

| NAS | Target Cone (mg/mL) | Actual Cone (mg/mL) | SD (mg/mL) | Viscosity (cP) | SEC (% Mono) | IEC (% Main p.) |
|---|---|---|---|---|---|---|
| Diethylene glycol monoethyl ether | 180 | 196.7 | 11.8 | 20 | | |
| | 230 | 238.3 | 6.2 | - | 93.4 | 64.7 |
| | 280 | 258.3 | 2.4 | 30 | | |
| Isosorbide dimethyl ether | 180 | 186.7 | 8.5 | - | | |
| | 230 | 220.0 | 4.1 | 28 | 89.6 | 61.9 |
| | 280 | 231.7 | 4.7 | - | | |
| Glycofurol | 180 | 188.3 | 4.7 | - | | |
| | 230 | 210.0 | 7.1 | 60 | 88.1 | 61.7 |
| | 280 | 241.7 | 4.7 | - | | |
| Ethyl oleate | 180 | 157.3 | 6.8 | - | | |
| | 230 | 223.3 | 20.5 | 50 | 94.9 | 64.2 |
| | 280 | 237.3 | 1.9 | - | | |
| Triacetin | 180 | 183.3 | 6.2 | - | | |
| | 230 | 193.3 | 6.2 | 70 | 95.0 | 64.2 |
| | 280 | 283.3 | 8.5 | 100 | | |
| PBS | 200 | 213 | 3.2 | 275 | 95.5 | 66.2 |

Dissociation of VA2 RPC SD powder in PBS 10 mM at 213 mg/mL resulted in very viscous solution (275 cP, Table 4). Suspension of VA2 RPC SD powder in NAS resulted in lower viscosity compared to PBS ranging from 20 to 70 cP for concentrations ranging from 193 to 223 mg/mL (Table 4). Protein stability evaluation after a short incubation time with the NAS showed an increase in the HMWS within Glycofurol and IDME. Protein aggregation was also observed within Triacetin, Transcutol and EO but at a lower extent (Table 4). Results from IEC also showed higher stability within Triacetin, Transcutol and EO compared to Glycofurol and IDME. Even though there is a reduction in the viscosity, injectability of the solutions was not possible through a 26G needle due to a large particle size.

### Example 3: Universality of the Concept

### 3.1 Universality of the Concept Using Different Proteins

Feasibility of RPC was evaluated using different protein formats (mAbs, bispecific crossmAb, cytokine fusion mAb, DutaFab) and dextran sulfate as the complexing agent at 1:1 mole-charge ratio.

The inventors have used dextran sulfate with 40 kDa molecular weight as the complexing agent. It is reported that DS has an average of two negative charges per monomer, corresponding to 240 negative charges per mole polymer. The number of positive charges of each protein was calculated from the amino acid sequence and are summarized in Table 5. Accordingly, the determined protein:DS complexation weight ratios corresponding to a 1:1 mole-charge ratio between each protein and DS are summarized in Table 5.

TABLE 5

Weight ratios between proteins and the complexing agent, dextran sulfate (DS), corresponding to 1:1 mole-charge ratio, used to prepare reversible protein complexes.

| Proteins | MW | Total (+) | Weight ratio | |
|---|---|---|---|---|
| | (kDa) | charges | Protein | DS |
| VEGF-Ang 2 | 146 | 146 | 1 | 0.167 |
| aSyn-mAb | 145 | 152 | 1 | 0.175 |
| FAP-OX40 | 195 | 188 | 1 | 0.160 |
| VEGF-PDGF DutaFab | 48 | 52 | 1 | 0.181 |
| Bevacizumab | 149 | 150 | 1 | 0.168 |
| Pertuzumab | 148 | 148 | 1 | 0.167 |
| Gantenerumab | 146 | 156 | 1 | 0.178 |

The study showed that RPC formation was possible with the different proteins tested corresponding to different protein formats (mAbs, bispecific crossmAbs, fusion mAbs, DutaFabs) with percentage complexation ranging from 96.1±1.4% to 99.5±0.3% (FIG. 8). The reversibility of the concept was also confirmed with regards to the different protein formats with percentage dissociation ranging from 96.5±1.6% to 105.4±0.8% in PBS 10 mM. Both complexation and dissociation were instantaneous at room temperature. RPC concept is hence applicable to a range a wide range of biologics.

Protein complexation with DS followed by dissociation in PBS 10 mM did not affect the stability of the protein. SEC and IEC results showed no significant difference between the percentages of monomers and main peaks obtained with the different proteins tested before and after complexation and dissociation (Table 6).

TABLE 6

Percentage monomer by SEC and main peak by IEC obtained with the different proteins before complexation and after complexation-dissociation. * Analytical methods not available.

| Proteins | % Monomer by SEC | | % Main Peak by IEC | |
|---|---|---|---|---|
| | Before complexation | After dissociation | Before complexation | After dissociation |
| VEGF-Ang 2 | 95.2 | 95.3 | 67.2 | 66.7 |
| aSyn-mAb | 99.0 | 98.6 | * | * |
| FAP-OX40 | * | * | * | * |
| VEGF-PDGF DutaFab | 99.1 | 98.8 | 88.7 | 86.9 |
| Bevacizumab | 97.2 | 97.5 | 71.3 | 69.8 |
| Pertuzumab | 99.4 | 98.6 | 62.1 | 61.5 |
| Gantenerumab | 97.7 | 98.4 | 49.0 | 49.1 |

### 3.2 Universality of the Concept Using Different Complexing Agents

Feasibility of the RPC was also evaluated using different complexing agents (CA) including dextran sulfate (DS), chondroitin sulfate (CS), SDS and sodium taurocholate (ST), with a bispecific cross mAb (VEGF-Ang2) as the protein model. Protein solution was prepared at 5 mg/mL and complexing agents’ solutions were prepared at 50 mg/mL in histidine buffer 20 mM pH 5.0, complexation was performed at different prot:CA mole-charge ratios.

The optimized Protein (VEGF-Ang2) to the complexing agent’s mole-charge ratios and the corresponding volume ratios are summarized in Table 7. Typically, with VEGF-Ang2 and DS, the inventors previously assessed 100 % complexation and dissociation at 1:1 mole-charge ratio. The ratio optimization showed that the same complexation-dissociation efficiency could be reached even at 1:0.6 ratio, corresponding to the minimal DS to be added for a total complexation of the protein. This suggests that the theoretical charge calculation method based on the protein sequence overestimated the number of positive charges available for complexation by DS, thus the actual amount of DS needed to provide 100% charge neutralization is lower than calculated. It is important to note that the inventors were considering the total number of positive charges distributed over the protein and not the protein net charge, as the protein net charge would underestimate the number of positive charges by charge addition.

TABLE 7

Optimized mole-charge and weight ratio between VEGF-Ang2 and different complexing agents (CA) used to prepare reversible protein complexes (RPC).

| Complexing agent (CA) | Mole-charge ratio | | Weight ratio | |
|---|---|---|---|---|
| | Protein | CA | Protein | CA |
| Chondroitin sulfate (CS) | 1 | 0.2 | 1 | 0.1 |
| Dextran sulfate (DS) | 1 | 0.6 | 1 | 0.1 |
| Sodium dodecyl sulfate (SDS) | 1 | 0.7 | 1 | 0.2 |
| Sodium taurocholate (ST) | 1 | 4.0 | 1 | 2.0 |

When adding DS, SDS and CS to the protein solution, complexation occurs at pH 5.0 (~ 100%). When using ST, complexation occurs only after adjusting the pH of the buffer solution to 4.0; moreover, 3 hours incubation time are needed to reach 100% complexation (FIG. 9).

Dissociation of the RPC formed with DS or CS is instant and complete. Dissociation of RPC formed using SDS and ST require more time; the use of PBS with a higher ionic strength (100 mM) accelerated the dissociation process (96.1% and 89.4%, respectively for SDS and ST, FIG. 5).

SEC analysis showed a good stability of protein after complexation and dissociation when using DS and CS. However, ST and even more, SDS, significantly degraded the protein. These results were also confirmed by IEC (Table 8).

TABLE 8

Percentage monomer by SEC and main peak by IEC of a bispecific mAb obtained before complexation and after complexation-dissociation using different complexing agents.

| Complexing agent (CA) | % Monomer by SEC | | % Main Peak by IEC | |
|---|---|---|---|---|
| | Before complexation | After dissociation | Before complexation | After dissociation |
| Chondroitin sulfate | 97.3 | 96.7 | 66.1 | 65.2 |
| Dextran sulfate | | 95.2 | | 66.7 |
| Sodium dodecyl sulfate | | 21.1 | | 61.1 |
| Sodium taurocholate | | 90.1 | | 62.9 |

### Example 4: Robustness of the Concept

Robustness of the RPC formation was evaluated along a range of protein concentration, buffer type, pH and ionic strength to determine the optimal conditions for RPC formation.

### 4.1 Effect of Protein Concentration

RPC formation was evaluated using protein concentrations ranging from 1 to 100 mg/mL in histidine buffer. VEGF-Ang2 dialyzed stock solution (130 mg/mL) was diluted to 100, 50, 40, 30, 25, 20, 5 and 1 mg/mL in histidine buffer 20 mM pH 5.0. The corresponding volume of DS (50 mg/mL) was added to 1 mL protein solution of every concentration at 1:1 mole charge ratio (1:0.167 weight ratio). Percentage complexation was evaluated after centrifugation and percentage dissociation was evaluated in PBS 10 mM.

Percentages of complexation and dissociation of VA2 and DS were determined at different protein concentrations (FIG. 10). Results showed that complexation occurs at any protein concentration, from 1 mg/mL to at least 100 mg/mL (highest concentration evaluated). Although, percentage complexation ranged from 96.7±2.4% to 99.5±0.0% at protein concentration ranging from 1 mg/mL to 40 mg/mL; complexation at concentrations higher than 40 mg/mL are limited because of the high viscosity associated with the high protein concentration, hindering the complexing agent from spreading over the solution and reaching every protein molecule to achieve a homogenous protein complexation.

Percentages of dissociation of the complexes formed at protein concentration from 1 mg/mL to 40 mg/mL ranged from 98.1±2.3% to 82.3±5.4%, with lower dissociation percentages at higher concentrations. Thus, the optimal protein concentration range for complete complexation and dissociation is defined to be from 1 mg/mL to 5 mg/mL (FIG. 10).

Initial protein concentration used for complexation was also found to have an effect on the final RPC particle size distribution (Table 9 and FIG. 11).

TABLE 9

RPC particle size after complexation with DS at different protein concentrations.

| Protein concentration | Mean (µm) | Median (µm) | SD (um) |
|---|---|---|---|
| 1 mg/mL | 7.8 | 7.5 | 2.1 |
| 5 mg/mL | 11.1 | 8.0 | 13.8 |
| 20 mg/mL | 21.6 | 19.4 | 10.3 |

### 4.2 Effect of Buffer Strength and pH on Complexation

RPC formation was evaluated using a range of histidine buffers with ionic strengths ranging from 5 to 50 mM and pH ranging from 1 to 7; and within citrate buffer 10-20 mM pH 5.0 at 5 mg/mL protein concentration and 1:1 mole-charge ratio with DS. RPC formation was also evaluated using ultra pure water (MilliQ water) as medium. Protein stock solution was dialyzed against ultra pure water (MilliQ water) then diluted to 5 mg/mL in ultra pure water (MilliQ water). The complexation was prepared by mixing the protein solution with the DS (50 mg/mL in ultra pure water (MilliQ water)) solution at 1:1 mole-charge ratio. Percentage complexation was evaluated after centrifugation and percentage dissociation was evaluated in PBS 10 mM.

Percentage complexation was evaluated within histidine buffer 20 mM at different pH ranging from 1 to 7 (FIG. 12). Their corresponding percentage dissociation were determined in PBS.

Percentages complexation ranged from 98.6±1.0% to 99.2±0.9% from pH 1 to 5.5, 93.5±0.2% at pH 6 and 1.3±0.4% at pH 7. The corresponding percentages dissociation ranged from 98.1±4.6 % to 109.2±3.8% (FIG. 12). Thus, the optimal buffer pH range for complexation is from 4.5 to 5.5 in order to ensure complete complexation and protein stability (strong acidic pH may degrade protein).

On the other hand, percentage complexation was evaluated in histidine buffer pH 5 at different ionic strengths ranging from 5 mM to 50 mM (FIG. 13). Their corresponding percentage dissociation were determined in PBS.

Percentages complexation ranged from 98.6±1.0% to 99.9±0.1% for ionic strengths ranging from 20 mM to 50 mM, 82.2±0.2% at 10 mM and 66.3±0.3% at 5 mM. The corresponding percentages dissociation ranged from 98.9±1.4% to 109.9±0.9% (FIG. 13). Although, RPC formulation with histidine buffer 50 mM showed a precipitation of the particles followed by the formation of a gel-like depot. Thus, the optimal buffer ionic strength range for complexation is from 20 mM to 30 mM in order to ensure complete complexation and formulation stability.

The inventors mainly used histidine buffer in this study, although, complexation also occurs using other buffers including citrate buffer 20 mM pH 5 with a percentage complexation of 97.2±2.0% and a percentage dissociation of 108.0±1.1%.

### 4.3 Effect of Buffer Strength, pH and Volume on Dissociation

Complex dissociation was evaluated using different media including PBS 10 mM (pH 7.4, NaCl 137 mM), phosphate buffer 10 mM (pH 7.4) and histidine buffer 20 mM containing saline (pH 5.0, NaCl 137 mM). RPC suspension was diluted in the corresponding media to 1 mg/mL, vortexed then dissociation was evaluated by visual inspection of the samples; when the solution is clear, protein content was measured by UV to determine the percentage of dissociation.

PBS (10 mM pH 7.4) volume needed for total instantaneous dissociation of RPC 5 mg/mL was evaluated at different RPC:PBS dilution ratios; 1:4, 1:2, 1:1.5 and 1:1 by mixing 100, 100, 200, 250 µL RPC with respectively 400, 300, 300, 250 µL PBS 10 mM. Percentage disscociation and pH of the final solutions were determined.

Different buffer ionic strengths and pH were tested to dissociate RPC particles. RPC particles dissociate instantly and completely within PBS resulting in a clear solution. RPC dilution in phosphate buffer 10 mM pH 7.4 resulted in a turbid solution due to incomplete dissociation of the RPC particles (~ 50% dissociation); addition of NaCl enabled total dissociation of the RPC particles. Dissociation of RPC particles in histidine buffer 20 mM pH 5.0 containing saline was even lower (~17%), where addition of NaCl also enabled total dissociation of the RPC particles. Thus, the best buffer for complete RPC dissociation is PBS 10 mM pH 7.4 (100 mM can also be used in some cases to accelerate the dissociation). In terms of PBS volume, a ratio PBS:RPC of at least 2:1 is required for total dissociation of RPC particles, resulting in pH increases to at least pH 6.5 in the formulation for total dissociation.

### 4.4 Effect of Excipients

The effect of generic excipients used in protein formulations on the RPC formation was evaluated by adding sucrose and/or surfactants to the formulation buffer. Sucrose, polysorbate 20 and/or poloxamer 188 were added to the protein solution diluted to 5 mg/mL in histidine buffer 20 mM pH 5.0 (formulations F1 to F6, Table 10) prior complexation with DS (1:1 mole-charge ratio) then the corresponding percentages of complexation and dissociation were determined.

TABLE 10

Additional excipients to RPC formulations in histidine buffer.

| Excipient | F1 | F2 | F3 | F4 | F5 | F6 |
|---|---|---|---|---|---|---|
| Sucrose | - | 240 mM | - | - | 240 mM | 240 mM |
| Polysorbate 20 | - | - | 0.05% | - | 0.05% | - |
| Poloxamer 188 | - | - | - | 0.05% | - | 0.05% |

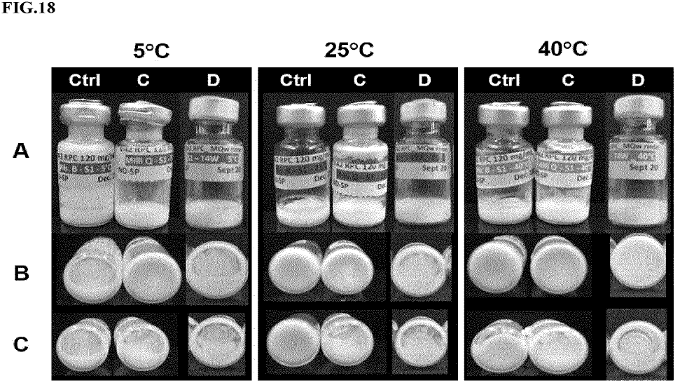

The presence of the additives (sucrose, polysorbate 20 and/or poloxamer 188) did not affect the RPC formation. Percentage complexation ranged from 99.2 to 99.6%. Additives did not affect RPC dissociation in PBS neither, with percentages ranging from 92.3% to 97.5% (FIG. 14,FIG. 15).

Complexation of VA2 with DS in ultra pure water (MilliQ water) instead of histidine buffer was not possible. Buffer is necessary for RPC formation (FIG. 15).

### Example 5: Short-Term Stability Study

A short-term study was conducted to evaluate the stability of the protein in RPC formulation (120 mg/mL) at different temperatures, in presence or absence of additives (sucrose, Poloxamer 188, polysorbate 20). RPC formulations (F1-F4) were formulated in histidine buffer and compared to formulation F5 where histidine buffer was exchanged with ultra pure water (MilliQ water) by dialysis.

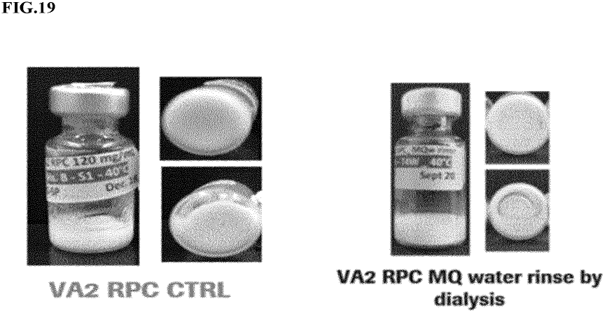

Lyophilization of RPC suspension at 60 mg/mL (F6) was successful and resulted in a homogenous cake that was easily reconstituted (and dissociated) to a final concentration of 120 mg/mL in PBS resulting in a transparent solution (FIG. 17).

In order to evaluate the feasibility of freeze drying and reconstitution of RPC suspensions, F6 was up concentrated to 60 mg/mL, 2 mL were filled into 6-mL vials then samples were placed in the lyophilizer for freeze drying. F6 is meant to be resonstituted in 0.85 mL PBS for a final concentration of 120 mg/mL VA2, 240 mM sucrose and 0.05% PS 20.

Stability of the protein in RPC formulations was compared to the standard protein solution (F7; drug product liquid solution 120 mg/mL).

0.5 mL of formulations F1-F5 and F7 were filled into 2-mL glass vials then stored with the lyophilized samples in the stability chambers (5, 25 and 40° C.) for four weeks. Composition of the different formulations is listed in Table 11.

Stability of the formulations was monitored at different time points in terms of visual aspect, protein content (UV), physical and chemical stability (SEC, IEC) and viscosity.

TABLE 11

Composition of the different formulations prepared for the stability study.

| Formulation | Buffer | Sucrose (mM) | NaCl (mM) | DS (mM) | Methionine (mM) | Polysorbate 20 (% w/v) | Poloxamer 188 (% w/v) |
|---|---|---|---|---|---|---|---|
| F1 | Histidine 20 mM pH 5.0 | - | - | 0.5 | - | - | - |
| F2 | | 240 | - | 0.5 | - | - | - |
| F3 | | 240 | - | 0.5 | - | 0.05 | - |
| F4 | | 240 | - | 0.5 | - | - | 0.05 |
| F6 | | 120 | - | 0.25 | - | 0.025 | - |
| F5 | Ultra pure water (MilliQ water) | - | - | 0.5 | - | - | - |
| F7 | Histidine acetate 20 mM pH 5.8 | 160 | 25 | - | 7 | 0.04 | - |

RPC formulations up-concentrated to 120 mg/mL (F1-F4) were liquid suspensions at initial time and no change was observed after 4-weeks storage of the different formulations at 5° C. However, after 4-weeks storage at 25° C. or 40° C., few visual changes were observed including the formation of a hard-cake, which in some formulations formed a shrinked pellet with or without a gel-aspect (FIG. 16). These hard cakes are irreversible, a magnetic stirrer and a vortex are needed to resuspend the RPC particles and reconstitute the suspension. Another interesting observation was during dissociation in PBS. After 4-weeks storage at 5° C. and 25° C., RPC particles dissociated completely; however, dissociation of RPC particles in formulations stored at 40° C. was partial forming a turbid solution upon dilution in PBS.

Interestingly, the hard cakes observed in F1-F4 formulations after 4-weeks storage et 25° C. and 40° C. were not observed in F5.

### 5.1 Protein Content Recovery After Dissociation

Protein concentration measured in the different RPC suspensions (F1-F5) following dissociation in PBS showed 92.7% to 107.0% recovery of the protein content after 4-weeks storage at 5° C. and 25° C. However, after 4-weeks storage at 40° C., protein content recovery ranged from 8.7% to 16.8%, resulting from incomplete dissociation of RPC particles (Table 12).

Total protein content was recovered following reconstitution of RPC lyophilisates (F6) in PBS after 4-weeks storage at 5, 25 and 40° C. with percentage recovery ranging from 101.1±1.6 to 105.0±0.4 (Table 12). Lyophilisation of RPC suspension could solve the incomplete dissociation observed after storage of RPC suspension at 40° C.

Protein content in F7 remains stable along the storage period at all temperatures (Table 12).

TABLE 12

Initial protein concentration and protein content recovery after 4-weeks storage of VA2 RPC formulations (F1-F5) and VA2 control solution (F6) at different temperatures.

| Initial | | 4 weeks 5° C. | | 4 weeks 25° C. | | 4 weeks 40° C. | |
|---|---|---|---|---|---|---|---|
| Conc. mg/mL | | Conc. Recovery mg/mL % | | Conc. Recovery mg/mL % | | Conc. Recovery mg/mL % | |
| F1 | 127.0 ± 2.2 | 124.7±1.2 | 98.2±0.9 | 117.7±1.2 | 92.7±0.9 | 11.0±0.8 | 8.7±0.6 |
| F2 | 121.7 ± 1.2 | 124.3±2.1 | 102.2±1.7 | 122.3±2.9 | 100.5±2.4 | 20.0±4.2 | 16.4±3.5 |
| F3 | 122.7±0.9 | 131.3±1.7 | 107.0±1.4 | 128.7±5.8 | 104.9±4.7 | 14.7±0.5 | 12.0±0.5 |
| F4 | 128.0±0.8 | 136.7±1.7 | 106.8±1.3 | 135.7±2.1 | 106.0±1.6 | 16.3±1.7 | 12.7±1.3 |
| F5 | 115.3±0.5 | 113.0±0.8 | 98.0±0.8 | 108.0±2.2 | 93.6±1.5 | 19.3±3.1 | 16.8±2.7 |
| F6 | 126.3±1.2 | 131.0±0.8 | 103.7±1.0 | 127.7±0.9 | 101.1±1.6 | 132.7±0.9 | 105.0±0.4 |
| F7 | 112.7±0.5 | 118.0±1.4 | - | 118.7±1.2 | - | 118.0±2.4 | - |

### 5.2 Protein Stability

SEC and IEC analysis of the protein in RPC formulations (F1-F4) after 4-weeks storage showed a good stability at 5° C. following dissociation in PBS. After 4-weeks storage at 25° C., 1.9% to 2.5% loss in the monomer was observed by SEC and 5.1% to 5.7% loss in the main peak was observed by IEC. After 4-weeks storage at 40° C., 22.0% to 28.8% loss in the monomer was observed by SEC and 41.6% to 45.7% loss in the main peak was observed by IEC (Table 13).

SEC analysis clearly showed that VA2 RPC suspension is not stable in ultra pure water (MilliQ water) (F5) as 8.7% loss in the monomer was observed after 4-weeks at 25° C. and 55.4% at 40° C.

No significant difference was observed before and after freeze drying process (F6) with percentages monomer obtained by SEC analysis of 96.5% and 96.8%, respectively. Follow up of the percentage monomer in RPC lyophilizate showed stable monomer after at least 4-weeks at 5° C. and 25° C.; 1.5% loss in the monomer was observed at 40° C. Similarly, no significant difference was observed by IEC analysis before and after freeze drying process with percentages main peak of 66.1% and 66.3%, respectively. Follow up of the percentage main peak in RPC lyophilizate showed a stable main peak after 4-weeks at 5° C., 1.7% loss at 25° C. and 5.4% loss at 40° C.

Protein percentage of monomer by SEC in control formulation (F7) remains stable after 4-weeks storage at all temperatures; percentage main peak by IEC showed good stability at 5° C. after 4-weeks storage, 2.4% and 22.9% loss in the main peak at 25° C. and 40° C., respectively.

### 5.3 Viscosity

Viscosity measurements showed no significant difference between the viscosities of RPC suspensions in histidine buffer (F1-F4); the different excipients seem not to contribute significantly to the final RPC viscosity. Overall, viscosity of VA2 formulations was lower in RPC suspensions compared to the control drug product solution (F7) with 10-12 cP versus 20 cP, respectively. There is a clear drop in the viscosity after exchanging histidine buffer with ultra pure water (MilliQ water) (F5), from 10 cP to 4 cP. After reconstitution of the lyophilized RPC suspension ion PBS (F6), viscosity increased slightly compared to RPC suspension (16 cP), however, still inferior to the control (F7) (Table 13).

TABLE 13

Initial viscosity and mean percentage monomer by SEC and percentage main peak by IEC after 4-weeks storage of VA2 RPC and control formulations (120 mg/mL) at different temperatures.

| Dosage form | | Viscosity (cP) | SEC (% monomer) | | | | IEC (% main peak) | | | |
|---|---|---|---|---|---|---|---|---|---|---|
| | | | Initial | 4-weeks | | | Initial | 4-weeks | | |
| | | | | 5° C. | 25° C. | 40° C. | | 5° C. | 25° C. | 40° C. |
| F1 | RPC suspension Histidine buffer | 10 | 95.1 | 96.0 | 92.7 | 66.2 | 66.2 | 68.1 | 61.1 | 20.5 |
| F2 | | 11 | 95.2 | 96.1 | 93.3 | 73.2 | 66.4 | 68.2 | 61.1 | 24.8 |
| F3 | | 12 | 95.1 | 96.3 | 92.9 | 71.0 | 66.8 | 68.1 | 61.1 | 23.2 |
| F4 | | 11 | 95.4 | 96.4 | 92.9 | 69.8 | 66.5 | 68.3 | 61.1 | 23.7 |
| F5 | RPC suspension Ultra pure water (MilliQ water) | 4 | 95.6 | 95.1 | 86.4 | 39.7 | * | * | * | * |
| F6 | RPC suspension Lyophilized | 16** | 96.8 | 96.8 | 96.5 | 95.3 | 66.3 | 66.0 | 64.6 | 60.8 |
| F7 | Control solution | 20 | 96.0 | 96.7 | 96.4 | 96.0 | 67.9 | 68.2 | 65.5 | 44.9 |

*Analysis not performed.** After reconstitution in PBS.

### Example 6: Dialysis of RPCs

In previous examples “hard-cake” formation has been observed upon storage of certain RPC samples at 25° C. and 40° C. This phenomenon affects visual aspect of the samples (the sample can appear “shrunken”) and complete resuspension of the particles is no longer possible, even if agitation is applied. Exchanging initial sample buffer (20 mM histidine buffer pH 5) with identical but fresh buffer or ultrapure water has resulted in improved sample behaviour at equal storage conditions. Buffer exchange has been performed by means of centrifugation where the protein-polymer complexes (RPC particles) had been sedimented, the supernatant was removed and the complexes in precipitate were then resuspended in fresh buffer or ultrapure water. Analyses of the samples have shown comparable particle size of RPC complexes in fresh histidine buffer and ultrapure water (i.e. in µm range) and lower physical stability of dissociated protein in ultrapure water when analyzed by size exclusion chromatography.

The aim of the subsequent experiments was to explore alternative methods for buffer exchange (e.g. dialysis) and further investigate as well as confirm improved visual aspect of samples treated in this manner.

### Methods

VEGF-Ang 2 (VA2) protein stock solution was prepared in 20 mM histidine buffer (pH 5) at a concentration of 5 mg/mL. For formation of protein-polymer complexes, 50 mg/mL complexing polymer (dextran sulfate sodium salt) solution in 20 mM histidine buffer (pH 5) was gradually added to the protein at constant mixing on a magnetic stirrer.

The prepared samples were always assessed for the % of complexation which was found to be > 98%.

In the next step, complexed solution was split into 3 parts with equal volumes - one part was dialysed against ultrapure water, another against fresh histidine buffer and the latter served as control and was not further manipulated. The dialysis was carried out using dialysis cassettes or tubings with MWCO of 10-100 kDa.

After dialysis, the samples were recovered and the protein concentration was adjusted to 120 mg/mL by means of centrifugation. The precipitate containing protein-polymer complexes was collected and the supernatant discarded.

The obtained samples were filled in 2 mL glass vials, stoppered, crimped and stored at different conditions (i.e. 5° C., 25° C., 30° C. or 40° C.).

At predefined time points they were characterized with regard to visual aspect and particle size prior dissociation of protein-polymer complexes in 10 mM phosphate buffer saline (pH 7.4) by means of laser diffraction or dynamic light scattering techniques (depending on the expected particle size). After dissociation, size and charge variants of the protein were determined by size exclusion and ion exchange chromatography respectively.

### Results

“Hard-cake” formation has not been observed for dialysed samples (either dialysed against ultrapure water or fresh histidine buffer) when stored for 1 month at any of the storage conditions. For samples dialysed against ultrapure water, the suspension appeared more liquid and lower amounts of particles have sedimented, whereas samples dialysed against histidine buffer resulted as more “pasty” and dense. Full resuspension was possible by agitation. In contrast to that, “hard-cake” formation has been detected in control samples stored at 25° C. and 40° C. (FIG. 18 and FIG. 19).

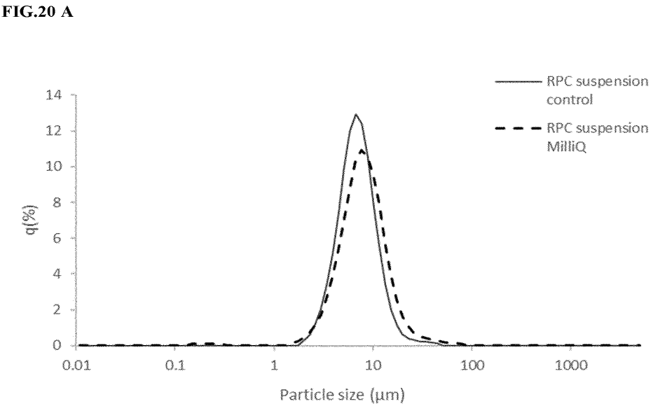

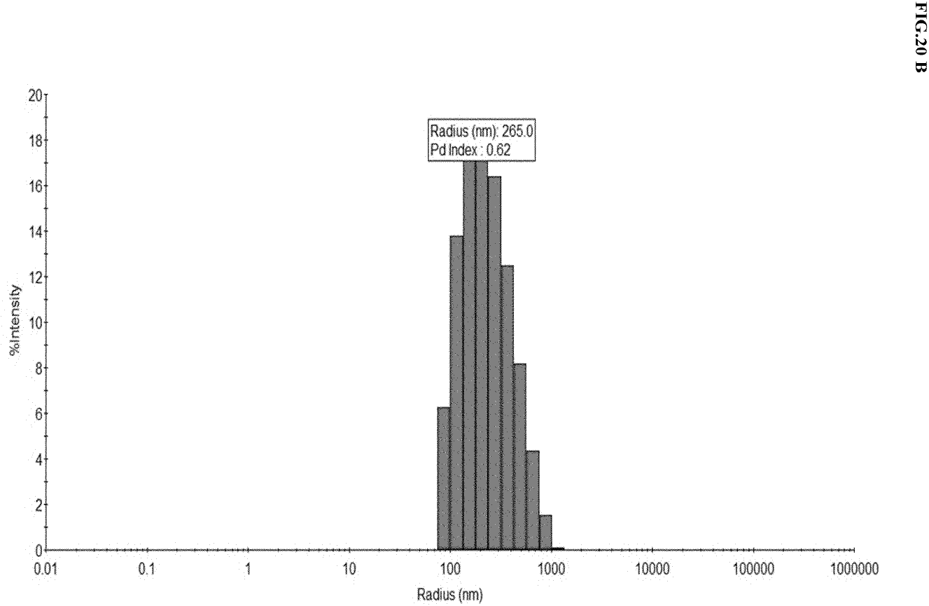

Surprisingly, for samples dialysed against ultrapure water, the particle size measured has been significantly lower than for samples in histidine buffer (control or dialysed against fresh buffer). For the first time, it has been in nm range, whereas for the latter (as previously measured and reported) in µm range (FIGS. 20A-20B). Additionally, the particle size of RPC when buffer was exchanged to ultrapure water by means of centrifugation has also been in µm range, which may imply that buffer exchange by means of dialysis results in better “rinsing” of the particles and more efficient buffer exchange.

Incomplete dissociation of complexes in samples stored at 40° C. was still observed, meaning full recovery of complexed protein was not possible (Table 14).

Stability of samples dialysed against ultrapure water was lower in comparison to the control (Table 14).

TABLE 14

Percentage monomer by SEC and main peak by IEC obtained for RPC suspension control and RPC suspension dialyzed against ultra pure water (MilliQ).

| Dosage form | SEC (% monomer) | | | | IEC (% main peak) | | | |
|---|---|---|---|---|---|---|---|---|
| | Initial | 4-weeks | | | Initial | 4-weeks | | |
| | | 5° C. | 25° C. | 40° C. | | 5° C. | 25° C. | 40° C. |
| RPC suspension Control | 97.5 | 97.6 | 96.5 | * | 73.5 | 73.9 | 66.1 | * |
| RPC suspension MilliQ | 97.2 | 97.9 | 91.3 | * | 72.6 | 73.3 | 60.7 | * |

* Analysis not performed 49962115vl

1. A method for producing a composition comprising reversible protein complexes (RPCs), the method comprising the steps of:
a) contacting a protein and a complexing agent in a buffer solution,
wherein the complexing agent is dextran sulphate or chondroitin sulphate, and
wherein the protein and the complexing agent have opposite net charges when comprised in the buffer solution;
b) formation of RPCs between the protein and the complexing agent in the buffer solution; and
c) obtaining a suspension comprising the RPCs formed in step (b).

2. The method according to claim 1, wherein the complexing agent is dextran sulphate, in particular dextran sulphate with 40 kDa molecular weight.

3. The method according to claim 1 or 2, wherein the pH of the buffer solution is adjusted to be lower than the isoelectric point of the protein.

4. The method according to any one of claims 1 to 3, wherein the pH of the buffer solution is adjusted to 2 to 5 pH units below the isoelectric point of the protein, in particular 3 pH units below the isoelectric point of the protein.

5. The method according to claim 1 or 2, wherein the buffer solution has a pH ranging from 1 to 6, in particular wherein the buffer solution has a pH ranging from 3 to 6, in particular wherein the buffer solution has a pH ranging from 4.5 to 5.5.

6. The method according to any one of claims 1 to 5, wherein the buffer solution has an ionic strength ranging from 20 to 50 mM, in particular wherein the buffer solution has an ionic strength ranging from 20 to 30 mM.

7. The method according to any one of claims 1 to 6, wherein the buffer solution comprises histidine or citrate as buffering agent.

8. The method according to any one of claims 1 to 7, wherein the buffer solution comprising the protein and the complexing agent is obtained by mixing a first solution comprising the protein and a second solution comprising the complexing agent.

9. The method according to claim 8, wherein the first solution comprising the protein and/or the second solution comprising the complexing agent comprises a buffering agent.

10. The method according to any one of claims 1 to 9, wherein the protein and the complexing agent are contacted at a mole-charge ratio ranging from 1:0.2 to 1:2, in particular wherein the protein and the complexing agent are contacted at a mole-charge ratio ranging from 1:0.2 to 1:1.

11. The method according to any one of claims 1 to 10, wherein the protein is contacted with the complexing agent in the buffer solution at a protein concentration ranging from 1-40 mg/mL, in particular from 1-5 mg/mL.

12. The method according to any one of claims 1 to 11, wherein the protein is an antibody, a growth factor, a hormone, a cytokine, an enzyme, or a fragment and/or fusion protein of any of the foregoing.

13. The method according to claim 12, wherein the antibody is an antibody, in particular wherein the antibody is a monoclonal antibody, a polyclonal antibody, a chimeric antibody, a multispecific antibody, an antibody fusion protein, an antibody-drug-conjugate or an antibody fragment.

14. The method according to any one of claims 1 to 13, wherein the complexing agent has a negative net charge when comprised in the buffer solution.

15. The method according to any one of claims 1 to 14, wherein the complexing agent comprises a hydrophobic moiety.

16. The method according to any one of claims 1 to 15, wherein the composition comprising the RPCs comprises at least one excipient.

17. The method according to claim 16, wherein the at least one excipient is added to the composition before and/or after the formation of the RPCs.

18. The method according to claim 17 or 18, wherein the at least one excipient is a stabilizer and/or a surfactant.

19. The method according to any one of claims 1 to 18, wherein the method comprises a further step of exchanging the liquid fraction of the suspension comprising the RPCs.

20. The method according to claim 19, wherein the liquid fraction of the suspension comprising the RPCs is exchanged by centrifugation of the suspension comprising the RPCs and resuspension of the sedimented RPCs in a buffer solution or water.

21. The method according to claim 19, wherein the liquid fraction of the suspension comprising the RPCs is exchanged by dialysis of the suspension comprising the RPCs against a buffer solution or water.

22. The method according to any one of claims 1 to 21, wherein the method comprises a further step of enriching the RPCs in the suspension to obtain an enriched RPC suspension.

23. The method according to claim 22, wherein enriching the RPCs in the suspension comprises the steps of:
a) centrifuging the suspension comprising the RPCs to obtain a supernatant and a precipitate comprising an enriched RPC suspension; and
b) removing the supernatant from the precipitate to obtain an enriched RPC suspension.

24. The method according to claim 22 or 23, wherein the liquid fraction of the enriched RPC suspension is at least in part replaced with a non-aqueous solvent during the enrichment step.

25. The method according to claim 24, wherein the non-aqueous solvent is triacetin, diethylene glycol monoethyl ether or ethyl oleate.

26. The method according to any one of claims 1 to 25, wherein the method comprises a further step of lyophilizing the suspension comprising the RPCs or the enriched RPC suspension to obtain a lyophilisate.

27. The method according to claim 26, wherein at least one cryoprotectant is added to the suspension comprising the RPCs or the enriched RPC suspension before the lyophilisation step.

28. The method according to claim 27, wherein the at least one cryoprotectant is selected from a group consisting of sugars, amino acids, methylamines, lyotropic salts, polyols, propylene glycol, polyethylene glycol and pluronics.

29. The method according to any one of claims 26 to 28, wherein the protein concentration of the suspension comprising the RPCs or the enriched RPC suspension is adjusted to 10 to 100 mg/mL, in particular to 40 to 80 mg/mL, prior to the lyophilisation step.

30. The method according to any one of claims 1 to 25, wherein the method comprises a further step of spray drying the suspension comprising the RPCs or the enriched RPC suspension to obtain a spray dried powder.

31. The method according to claim 30, wherein the protein concentration of the suspension comprising the RPCs or the enriched RPC suspension is adjusted to 1 to 10 mg/mL, in particular to 1 to 5 mg/mL, prior to the spray drying step.

32. The method according to claim 30 or 31, wherein the liquid fraction of the suspension comprising the RPCs or the enriched RPC suspension is exchanged prior to the spray drying step.

33. The method according to claim 32, wherein exchanging the liquid fraction of the suspension comprising the RPCs or the enriched RPC suspension reduces the concentration of at least one buffering agent, complexing agent and/or excipient in the suspension.

34. The method according to claim 33, wherein the suspension comprising the RPCs or the enriched RPC suspension is substantially free of buffering agent after exchanging the liquid fraction of the suspension.

35. The method according to claim 33 or 34, wherein the liquid fraction of the suspension is exchanged before the spray-drying step to obtain a mole-charge ratio between the protein and the complexing agent between 1:0.2 to 1:1, in particular between 1:0.4 to 1:0.8.

36. The method according to any one of claims 30 to 35, wherein spray drying is performed at an inlet temperature 115° C. and/or an outlet temperature of 48° C.

37. The method according to any one of claims 30 to 36, wherein spray drying is performed at a feed rate of 17 mL/min.

38. The method according to any one of claims 30 to 37, wherein the method comprises a further step of resuspending the spray dried powder in a non-aqueous solvent (NAS) to obtain an RPC-NAS suspension.

39. The method according to claim 38, wherein the non-aqueous solvent is at least one selected from a group consisting of: diethylene glycol monoethyl ether, ethyl oleate, triacetin, isosorbide dimethyl ether and glycofurol.

40. The method according to claim 39, wherein the spray dried powder is resuspended to obtain a RPC-NAS suspension with a protein concentration ranging from 50 to 300 mg/mL, in particular ranging from 100 - 250 mg/mL.

41. A composition comprising reversible protein complexes (RPCs), wherein the composition is obtained by the method according to any one of claims 1 to 40.

42. A composition comprising reversible protein complexes (RPCs), wherein the RPCs comprise a protein and a complexing agent, and
wherein the complexing agent is dextran sulphate or chondroitin sulphate.

43. The composition according to claim 42, wherein the complexing agent is dextran sulphate, in particular dextran sulphate with 40 kDa molecular weight.

44. The composition according to claim 42 or 43, wherein the protein has a positive net charge when comprised in the RPCs.

45. The composition according to claim 44, wherein the protein is an antibody, a growth factor, a hormone, a cytokine, an enzyme, or a fragment and/or fusion protein of any of the foregoing.

46. The composition according to claim 45, wherein the antibody is a monoclonal antibody, a polyclonal antibody, a chimeric antibody, a multispecific antibody, an antibody fusion protein, an antibody-drug-conjugate or an antibody fragment.

47. The composition according to any one of claims 42 to 46, wherein the complexing agent has a negative charge when comprised in the RPCs.

48. The composition according to any one of claims 42 to 47, wherein the complexing agent comprises a hydrophobic moiety.

49. The composition according to any one of claims 42 to 48, wherein the composition comprises at least one excipient.

50. The composition according to claim 49, wherein the at least one excipient is a stabilizer and/or a surfactant.

51. The composition according to any one of claims 42 to 50, wherein the protein has a higher melting temperature when comprised in the RPC compared to the uncomplexed protein.

52. The composition according to any one of claims 42 to 51, wherein the RPCs comprising the protein and the complexing agent dissociate at physiological pH and ionic strength.

53. The composition according to any one of claims 42 to 51, wherein the RPCs comprising the protein and the complexing agent dissociate in 10 mM to 100 mM PBS (pH 7.4, 137 mM NaCl) when diluted to a protein concentration of 0.1 to 10 mg/mL.

54. The composition according to any one of claims 42 to 53, wherein the composition is a suspension.

55. The composition according to claim 54, wherein the suspension is obtained with the method according to any one of claims 1 to 25.

56. The composition according to claims 54 or 55, wherein the protein concentration in the suspension ranges from 50 to 250 mg/mL, in particular wherein the protein concentration in the suspension ranges from 100 to 200 mg/mL.

57. The composition according to any one of claims 54 to 56, wherein the suspension comprises uncomplexed complexing agent.

58. The composition according to any one of claims 54 to 57, wherein the RPCs comprised in the suspension have a mean particle size ranging from 5 to 20 µm, in particular wherein the RPCs comprised in the suspension have a mean particle size ranging from 6 to 12 µm.

59. The composition according to any one of claims 54 to 57, wherein the RPCs comprised in the suspension have a mean particle size ranging from 100 to 4000 nm, in particular wherein the RPCs comprised in the suspension have a mean particle size ranging from 150 to 2000 nm.

60. The composition according to any one of claims 54 to 57, wherein the RPCs comprised in the suspension have a mean particle size ranging from 0.1 to 20 µm, in particular wherein the RPCs comprised in the suspension have a mean particle size ranging from 0.1 to 12 µm.

61. The composition according to any one of claims 54 to 60, wherein the suspension is injectable through a 26G needle.

62. The composition according to any one of claims 54 to 61, wherein the suspension is stable for at least 4 weeks at 4° C. and/or 25° C.

63. The composition according to any one of claims 54 to 62, wherein the suspension has a viscosity ranging from 2 to 20 cP, in particular ranging from 3 to 15 cP, when measured at 20° C.

64. The composition according to any one of claims 54 to 63, wherein the pH of the suspension is lower than the isoelectric point of the protein.

65. The composition according to any one of claims 54 to 64, wherein the pH of the suspension is 1 to 3 pH units lower than the isoelectric point of the protein, in particular wherein the pH of the suspension is 2 pH units lower than the isoelectric point of the protein.

66. The composition according to any one of claims 54 to 64, wherein the pH of the suspension ranges from 1 to 6, in particular wherein the pH of the suspension ranges from 4.5 to 5.5.

67. The composition according to any one of claims 54 to 66, wherein the suspension comprises a buffering agent.

68. The composition according to claim 67, wherein the buffering agent is histidine or citrate.

69. The composition according to any one of claims 54 to 68, wherein the suspension has an ionic strength ranging from 20 to 50 mM, in particular wherein the suspension has an ionic strength ranging from 20 to 30 mM.

70. The composition according to any one of claims 54 to 66, wherein the suspension is substantially free of buffering agents.

71. The composition according to any one of claims 54 to 70, wherein the suspension further comprises a non-aqueous solvent.

72. The composition according to claim 71, wherein the non-aqueous solvent is diethylene glycol monoethyl ether, triacetin or ethyl oleate.

73. The composition according to any one of claims 42 to 53, wherein the composition is a lyophilisate.

74. The composition according to claim 73, wherein the lyophilisate is obtained with the method according to any one of claims 26 to 29.

75. The composition according to claim 73 or 74, wherein the lyophilisate comprises a buffering agent.

76. The composition according to claim 75, wherein the buffering agent is histidine or citrate.

77. The composition according to any one of claims 73 to 76 wherein the lyophilisate comprises at least one cryoprotectant.

78. The composition according to claim 77, wherein the at least one cryoprotectant is selected from a group consisting of: sugars, amino acids, methylamines, lyotropic salts, polyols, propylene glycol, polyethylene glycol and pluronics.

79. The composition according to any one of claims 73 to 78, wherein the lyophilisate is stable for at least 4 weeks at 40° C.

80. The composition according to any one of claims 73 to 79, wherein the lyophilisate is reconstituted in a liquid to a protein concentration ranging from 50 to 250 mg/mL, in particular wherein the lyophilisate is reconstituted in a liquid to a protein concentration ranging from 100 to 200 mg/mL.

81. The composition according to claim 80, wherein the liquid is PBS.

82. The composition according to claim 80 or 81, wherein the resuspended lyophilisate has a viscosity ranging from 2 to 20 cP, in particular ranging from 10 to 20 cP.

83. The composition according to any one of claims 42 to 53, wherein the composition is a spray dried powder.

84. The composition according to claim 83, wherein the protein content of the spray dried powder is at least 40% by weight (w/w), at least 50% by weight (w/w), at least 60% by weight (w/w).

85. The composition according to claim 83 or 84, wherein the spray dried powder is obtained with the method according to any one of claims 30 to 37.

86. The composition according to any one of claims 83 to 85, wherein the spray dried powder comprises a buffering agent.

87. The composition according to claim 86, wherein the buffering agent is histidine or citrate.

88. The composition according to any one of claims 83 to 85, wherein the spray dried powder is substantially free of buffering agents.

89. The composition according to any one of claims 83 to 88, wherein the RPCs comprised in the spray dried powder have a mean particle size ranging from 5 to 50 µm, in particular ranging from 10 to 40 µm, in particular ranging from 20 to 35 µm.

90. The composition according to any one of claims 83 to 89, wherein the spray dried powder is re-suspended in a liquid to a protein concentration in the suspension ranging from 50 to 300 mg/mL, in particular wherein the spray dried powder is re-suspended in a liquid to a protein concentration in the suspension ranging from 100 to 250 mg/mL.

91. The composition according to claim 90, wherein the liquid is a non-aqueous solvent.

92. The composition according to claim 91, wherein the non-aqueous solvent is at least one selected from a group consisting of: diethylene glycol monoethyl ether, ethyl oleate, triacetin, isosorbide dimethyl ester and glycofurol, preferably diethylene glycol monoethyl ether, ethyl oleate or triacetin.

93. The composition according to any one of claims 90 to 92, wherein the reconstituted spray dried powder has a viscosity ranging from 10 to 100 cP, in particular ranging from 20 to 80 cP.

94. A pharmaceutical formulation comprising the composition according to any one of claims 41 to 93.

95. The pharmaceutical formulation according to claim 94, wherein the pharmaceutical formulation comprises the suspension according to any one of claims 54 to 72, the reconstituted lyophilisate according to any one of claims 80 to 82, or the re-suspended spray dried powder according to any one of claims 90 to 93.

96. The pharmaceutical formulation according to claims 94 or 95 for use as a medicament.

97. The pharmaceutical formulation according to any one of claims 94 to 96 for use in the treatment of an autoimmune disease, an immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, or atherosclerosis.

98. The pharmaceutical formulation for use according to claim 97, wherein the pharmaceutical formulation is administered subcutaneously, intramuscularly, transdermally, ocullarly, such as subconjunctivally, intracamerally, intravitreally, subretinally, or suprachoroidally, to the brain, such as intralumbarly, intrathecally, or intraventricularly, intra-articularly, or by inhalation.

99. Use of the pharmaceutical formulation according to claims 94 or 95 for the treatment of a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis.

100. Use of the pharmaceutical formulation according to claims 94 or 95 in the preparation of a medicament for the treatment of a disease selected from the group consisting of autoimmune disease, immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis.

101. A method of treating a subject suffering from a disease selected from the group consisting of an autoimmune disease, an immune dysregulation disease, carcinoma, sarcoma, glioma, melanoma, lymphoma, leukemia, chronic lymphocytic leukemia, follicular lymphoma, diffuse large B cell lymphoma, multiple myeloma, non-Hodgkin’s lymphoma, Alzheimer’s disease, type 1 or type 2 diabetes, amyloidosis, and atherosclerosis, the method comprising the steps of (a) producing the pharmaceutical formulation according claims 94 or 95; and (b) administering the pharmaceutical formulation to a subject in need thereof.

102. The method according to claim 101, wherein the pharmaceutical composition is administered subcutaneously, intramuscularly or transdermally, in particular wherein the pharmaceutical composition is administered subcutaneously.

103. A method of subcutaneous, intramuscular or transdermal administration of a pharmaceutical formulation, the method comprising the steps of (a) producing the pharmaceutical formulation according to claims 94 or 95; and (b) administering the pharmaceutical formulation to a subject by subcutaneous, intramuscular or transdermal delivery.
